SEQUENCE LISTING (13 sequences)

SEQ ID NO: 1 - protein, 473 aa, synthetic construct
  REGION 1..473: note = SARS-COV2 Nucleocapsid protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGPGPGNLVPMVATVGPGPGMLIPIAVGGALAGLVLIVLIAYLIGKKHCSYQDIL

SEQ ID NO: 2 - protein, 516 aa, synthetic construct
  REGION 1..516: note = SARS-CoV2 Nucleocapsid protein tagged with ETSD signal
MLLLPFQLLAVLFPGGNSEDYKDDDDKGGGSGGGSGGGSGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGPGPGNLVPMVATVGPGPGMLIPIAVGGALAGLVLIVLIAYLIGKKHCSYQDIL

SEQ ID NO: 3 - DNA, 1551 bp, synthetic construct
  misc_feature 1..1551: note = N-ETSD cargo in AdV. Seq I.(2)
  source 1..1551: mol_type = other DNA
atgctgctgctgcccttccagttgctggctgtcctctttcccggcggcaactccgaggattacaaggacgacgacgacaagggtggaggctctggaggtggctctggtggaggttccggtggcggatctatgagcgacaacggtccccagaatcaaagaaatgcgcccagaattacattcggcggcccttctgatagcactggctcaaatcaaaacggggagagaagcggagccaggtccaaacagcggagaccccaaggcctgcctaataacaccgcttcctggttcacagctctgacgcaacacggcaaggaggatctgaagtttccacggggtcagggcgtcccgattaacacgaactctagcccagatgaccaaatagggtactacagaagagcgacaaggcggatcagaggaggcgatggaaaaatgaaggatctgtcccctaggtggtatttctattacctgggcacaggccctgaagctgggttgccttacggcgcaaacaaagatggaattatatgggtggccaccgagggggcgttgaacaccccaaaggatcacatcggaacgaggaatcccgccaacaatgctgctatagtgctccaactgccacagggaacaaccctgcctaagggcttctacgccgaggggagccgcggtggcagccaggccagctccagaagttcctcccgcagccggaacagctctagaaacagcactcccggcagctccagagggacaagcccagccagaatggccggcaatggcggcgacgctgccctcgcacttctgttgcttgatcggctcaatcaactcgaaagcaaaatgtccggcaagggacaacaacagcaaggacagaccgttacaaaaaaaagcgccgccgaggctagcaagaagcccagacagaagcgaaccgcaacaaaggcctataatgtaacacaagcctttggaaggcggggacccgaacagacccagggaaattttggcgaccaggaactgatccggcaagggacagactataaacattggccacagatagcgcaatttgctccctccgcctccgccttctttggcatgtcaagaataggcatggaagtaactccttctggaacctggctgacgtacactggggcaatcaagttggatgataaggaccctaatttcaaggaccaagttattttgctcaacaagcatatagacgcctacaagactttcccgcctaccgaacctaaaaaggataagaagaagaaagcagacgagacccaggccctgcctcaacggcaaaagaagcagcaaactgtgacactcctgcccgccgctgacttggatgatttttcaaaacagctccaacagagtatgagcagcgccgatagcacccaagctggaccgggtccgggcaacctggtgccgatggtggcgaccgtgggtccaggaccgggtatgctgatccccatcgccgtgggcggggccctggccggcctcgtgctgatcgtccttatcgcctacctcatcggcaagaagcactgctcatatcaggacatcctgtga

SEQ ID NO: 4 - protein, 1282 aa, synthetic construct
  REGION 1..1282: note = spike protein
MFVFLVLLPLVSSYPYDVPDYAQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 3849 bp, synthetic construct
  misc_feature 1..3849: note = S-HA cargo in AdV. Seq I.(2)
  source 1..3849: mol_type = other DNA
atgttcgtttttctcgttctcctcccgcttgtgagcagctatccgtatgatgtgccggattatgcgcaatgtgtcaacctcaccacaaggacacagctccctcccgcatatacgaatagctttaccagaggcgtatactatcctgataaggtctttaggagctcagtactgcatagcactcaggatctcttcctgccgttcttcagtaatgttacttggtttcacgccattcatgtttccgggaccaatggcaccaaacggttcgataatccagtgcttcccttcaacgatggggtgtactttgccagcactgaaaaatctaatataattcggggatggattttcggaaccacactcgattccaagactcagtccctcttgatcgttaacaacgctactaatgttgtcattaaggtgtgtgagtttcagttctgcaacgaccctttcctgggtgtctactaccataaaaataacaagagctggatggagtccgaatttcgcgtctactcaagcgccaataattgcacttttgagtatgtgtcccagccctttttgatggatctggagggaaagcagggcaatttcaaaaatctgagagaattcgtttttaagaatatagatggatacttcaaaatctacagcaaacacacacccataaatcttgtgcgcgatcttccccagggcttcagcgcgttggaaccccttgttgacttgcccataggcatcaacattaccaggttccaaacgctgctcgccctccaccgcagctacttgacacccggggattccagctccggatggaccgccggcgccgcagcgtattatgtggggtacctgcaacccaggacatttttgctcaagtacaatgagaatgggaccatcacagatgcggtagactgtgcactggatccactcagcgaaactaaatgtaccctgaaaagctttaccgtggagaaaggaatctaccaaaccagcaacttcagggtccagcccactgaatccatcgttagatttccaaatataactaatttgtgtccatttggagaggtgttcaatgctacaaggttcgcgtctgtatacgcttggaaccggaagcgcatctcaaattgcgtggctgattatagcgttctttacaacagcgcttccttttccacgttcaagtgctatggtgtatccccgacaaagctgaatgacttgtgcttcaccaatgtgtatgcggattctttcgttattcgaggcgatgaagtcagacaaattgcgcctggccagaccggaaagattgccgactacaactataaactgccggacgactttactggttgcgtgatcgcttggaacagcaataatcttgatagtaaagttggaggaaactacaattacctctatagactgttcagaaagagcaacttgaagccattcgaacgggatatctctacggagatctatcaagctggcagcaccccctgcaatggtgtggaaggctttaattgttattttcctttgcagagctatggcttccaacctaccaacggagtgggctaccagccctacagagtggtggtgctcagctttgaactgctgcatgccccggccacagtttgcgggcccaaaaaaagcacgaatctggttaagaacaaatgcgtcaacttcaattttaatgggttgacaggtacaggcgtactgaccgaatccaacaaaaagttcctgccttttcagcagttcgggagagatatcgccgacactacagacgccgtcagggatccccaaacactcgaaattctggacatcacaccttgttccttcggcggggtatctgtgattactccgggcacaaataccagtaaccaggtagcggtgctttaccaggatgtcaactgtacggaagtacctgtcgctattcatgcggatcaactcactcctacctggagagtttattccactgggtccaacgtgtttcagacccgagccggctgcttgattggcgcggaacatgttaacaactcctacgaatgtgacatccctatcggagctggcatctgtgcttcctatcaaacgcaaacgaacagcccacggcgggccagatccgtagcctctcaaagcatcatcgcttatactatgtccttgggggctgaaaacagcgttgcctattccaacaatagcatcgctatccctaccaactttaccatttccgtgaccacagaaatactgccggtgagcatgacaaagacttctgtggactgtaccatgtatatatgcggcgatagcacagagtgttctaatttgctgctgcagtacggcagcttttgtacccaactcaacagagcacttacagggattgccgtcgagcaggataaaaacacccaggaggttttcgcccaggttaagcagatctacaagaccccaccaatcaaggatttcggcggcttcaatttttcccagatactgcccgatccttccaagccatccaaaaggagctttatagaggatctgctgttcaacaaggtgactctggccgacgctggctttatcaagcaatatggcgattgcctgggggatattgccgctagggaccttatctgcgctcaaaaattcaacggtcttaccgttctcccgcccctgctcaccgacgagatgatagcccagtacacgagcgcacttttggccggcacgataaccagcggctggacattcggtgccggggccgctcttcaaatcccctttgccatgcagatggcctacagatttaatgggataggcgtgacacaaaatgtcttgtatgaaaatcagaaactgattgcaaaccagtttaatagcgctattggcaagatccaagatagcctttcctccaccgcatccgctctgggaaagttgcaagacgtcgtgaatcaaaacgcccaagctctgaataccctcgtgaagcagcttagctccaactttggcgcgatatcctccgtgctgaacgatatcctgtccagattggacaaggtcgaggcagaagtccagatcgatagattgataaccggcagactccagtctctgcagacatatgtgactcagcagttgataagagcggccgaaatacgagcgtctgcaaatctcgcagcaacgaaaatgtcagagtgtgtattggggcaaagtaaaagagtagatttctgtggaaagggttaccatctgatgtcattcccccagtctgcaccacatggagtagtttttttgcatgtgacttatgtgcctgcccaggagaaaaatttcaccactgcacctgcgatctgtcatgacggcaaggcacatttccctagagaaggcgtcttcgtatcaaatggaacacactggtttgtaacccaaaggaacttttacgagccccaaattataactaccgacaacaccttcgtaagcggaaactgcgacgtcgttatagggatagtcaataatacggtctatgaccctcttcagccggaactggactcctttaaagaagaactggataagtacttcaagaaccatacgtctccggatgtggatctcggagatataagtggaatcaacgcaagcgtagtaaacattcagaaggagatagaccgactcaatgaggttgctaaaaacctgaacgaaagcttgatagacttgcaggagctgggtaagtacgaacagtacattaagtggccatggtatatctggttgggcttcatagcaggactcatagctatcgtcatggtgacaataatgctttgttgtatgaccagctgttgttcttgtctgaaaggctgctgcagctgtggcagctgttgtaaatttgacgaagatgattccgagcctgtgcttaagggcgtaaaactccactatacatga

SEQ ID NO: 6 - protein, 1298 aa, synthetic construct
  REGION 1..1298: note = Spike fusion construct prt
MFVFLVLLPLVSSYPYDVPDYAGGGSGGGSGGGSGGGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - DNA, 3897 bp, synthetic construct
  misc_feature 1..3897: note = spike fusion construct dna
  source 1..3897: mol_type = other DNA
atgttcgtttttctcgttctcctcccgcttgtgagcagctatccgtatgatgtgccggattatgcgggtggaggctctggaggtggctctggtggaggttccggtggcggatctcaatgtgtcaacctcaccacaaggacacagctccctcccgcatatacgaatagctttaccagaggcgtatactatcctgataaggtctttaggagctcagtactgcatagcactcaggatctcttcctgccgttcttcagtaatgttacttggtttcacgccattcatgtttccgggaccaatggcaccaaacggttcgataatccagtgcttcccttcaacgatggggtgtactttgccagcactgaaaaatctaatataattcggggatggattttcggaaccacactcgattccaagactcagtccctcttgatcgttaacaacgctactaatgttgtcattaaggtgtgtgagtttcagttctgcaacgaccctttcctgggtgtctactaccataaaaataacaagagctggatggagtccgaatttcgcgtctactcaagcgccaataattgcacttttgagtatgtgtcccagccctttttgatggatctggagggaaagcagggcaatttcaaaaatctgagagaattcgtttttaagaatatagatggatacttcaaaatctacagcaaacacacacccataaatcttgtgcgcgatcttccccagggcttcagcgcgttggaaccccttgttgacttgcccataggcatcaacattaccaggttccaaacgctgctcgccctccaccgcagctacttgacacccggggattccagctccggatggaccgccggcgccgcagcgtattatgtggggtacctgcaacccaggacatttttgctcaagtacaatgagaatgggaccatcacagatgcggtagactgtgcactggatccactcagcgaaactaaatgtaccctgaaaagctttaccgtggagaaaggaatctaccaaaccagcaacttcagggtccagcccactgaatccatcgttagatttccaaatataactaatttgtgtccatttggagaggtgttcaatgctacaaggttcgcgtctgtatacgcttggaaccggaagcgcatctcaaattgcgtggctgattatagcgttctttacaacagcgcttccttttccacgttcaagtgctatggtgtatccccgacaaagctgaatgacttgtgcttcaccaatgtgtatgcggattctttcgttattcgaggcgatgaagtcagacaaattgcgcctggccagaccggaaagattgccgactacaactataaactgccggacgactttactggttgcgtgatcgcttggaacagcaataatcttgatagtaaagttggaggaaactacaattacctctatagactgttcagaaagagcaacttgaagccattcgaacgggatatctctacggagatctatcaagctggcagcaccccctgcaatggtgtggaaggctttaattgttattttcctttgcagagctatggcttccaacctaccaacggagtgggctaccagccctacagagtggtggtgctcagctttgaactgctgcatgccccggccacagtttgcgggcccaaaaaaagcacgaatctggttaagaacaaatgcgtcaacttcaattttaatgggttgacaggtacaggcgtactgaccgaatccaacaaaaagttcctgccttttcagcagttcgggagagatatcgccgacactacagacgccgtcagggatccccaaacactcgaaattctggacatcacaccttgttccttcggcggggtatctgtgattactccgggcacaaataccagtaaccaggtagcggtgctttaccaggatgtcaactgtacggaagtacctgtcgctattcatgcggatcaactcactcctacctggagagtttattccactgggtccaacgtgtttcagacccgagccggctgcttgattggcgcggaacatgttaacaactcctacgaatgtgacatccctatcggagctggcatctgtgcttcctatcaaacgcaaacgaacagcccacggcgggccagatccgtagcctctcaaagcatcatcgcttatactatgtccttgggggctgaaaacagcgttgcctattccaacaatagcatcgctatccctaccaactttaccatttccgtgaccacagaaatactgccggtgagcatgacaaagacttctgtggactgtaccatgtatatatgcggcgatagcacagagtgttctaatttgctgctgcagtacggcagcttttgtacccaactcaacagagcacttacagggattgccgtcgagcaggataaaaacacccaggaggttttcgcccaggttaagcagatctacaagaccccaccaatcaaggatttcggcggcttcaatttttcccagatactgcccgatccttccaagccatccaaaaggagctttatagaggatctgctgttcaacaaggtgactctggccgacgctggctttatcaagcaatatggcgattgcctgggggatattgccgctagggaccttatctgcgctcaaaaattcaacggtcttaccgttctcccgcccctgctcaccgacgagatgatagcccagtacacgagcgcacttttggccggcacgataaccagcggctggacattcggtgccggggccgctcttcaaatcccctttgccatgcagatggcctacagatttaatgggataggcgtgacacaaaatgtcttgtatgaaaatcagaaactgattgcaaaccagtttaatagcgctattggcaagatccaagatagcctttcctccaccgcatccgctctgggaaagttgcaagacgtcgtgaatcaaaacgcccaagctctgaataccctcgtgaagcagcttagctccaactttggcgcgatatcctccgtgctgaacgatatcctgtccagattggacaaggtcgaggcagaagtccagatcgatagattgataaccggcagactccagtctctgcagacatatgtgactcagcagttgataagagcggccgaaatacgagcgtctgcaaatctcgcagcaacgaaaatgtcagagtgtgtattggggcaaagtaaaagagtagatttctgtggaaagggttaccatctgatgtcattcccccagtctgcaccacatggagtagtttttttgcatgtgacttatgtgcctgcccaggagaaaaatttcaccactgcacctgcgatctgtcatgacggcaaggcacatttccctagagaaggcgtcttcgtatcaaatggaacacactggtttgtaacccaaaggaacttttacgagccccaaattataactaccgacaacaccttcgtaagcggaaactgcgacgtcgttatagggatagtcaataatacggtctatgaccctcttcagccggaactggactcctttaaagaagaactggataagtacttcaagaaccatacgtctccggatgtggatctcggagatataagtggaatcaacgcaagcgtagtaaacattcagaaggagatagaccgactcaatgaggttgctaaaaacctgaacgaaagcttgatagacttgcaggagctgggtaagtacgaacagtacattaagtggccatggtatatctggttgggcttcatagcaggactcatagctatcgtcatggtgacaataatgctttgttgtatgaccagctgttgttcttgtctgaaaggctgctgcagctgtggcagctgttgtaaatttgacgaagatgattccgagcctgtgcttaagggcgtaaaactccactatacatga

SEQ ID NO: 8 - protein, 805 aa, synthetic construct [sequence omitted]

SEQ ID NO: 9 - protein, 594 aa, synthetic construct
  REGION 1..594: note = Soluble ACE2 mutant constructs
  VARIANT 4: note = any one of Q, or L
  VARIANT 7: note = any one of T, F, W, or Y
  VARIANT 10: note = any one of D, E, or L
  VARIANT 14: note = any one of H, E, F, K, M, W, Y, or A
  VARIANT 18: note = any one of D, E, M, or F
  VARIANT 335: note = any one of D or L
IEEXAKXFLXKFNXEAEXLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGXFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 10 - protein, 1298 aa, synthetic construct
  REGION 1..1298: note = SARS-CoV2 spike mutant (p35)
MFVFLVLLPLVSSYPYDVPDYAGGGSGGGSGGGSGGGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSAAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - DNA, 40866 bp, synthetic construct
  misc_feature 1..40866: note = p35 vector sequence
  source 1..40866: mol_type = other DNA
tattattgatgatgttaattaacatgcatggatcctacgtctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggatcgaattaattcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttatgttcgtttttctcgttctcctcccgcttgtgagcagctatccgtatgatgtgccggattatgcgggtggaggctctggaggtggctctggtggaggttccggtggcggatctcaatgtgtcaacctcaccacaaggacacagctccctcccgcatatacgaatagctttaccagaggcgtatactatcctgataaggtctttaggagctcagtactgcatagcactcaggatctcttcctgccgttcttcagtaatgttacttggtttcacgccattcatgtttccgggaccaatggcaccaaacggttcgataatccagtgcttcccttcaacgatggggtgtactttgccagcactgaaaaatctaatataattcggggatggattttcggaaccacactcgattccaagactcagtccctcttgatcgttaacaacgctactaatgttgtcattaaggtgtgtgagtttcagttctgcaacgaccctttcctgggtgtctactaccataaaaataacaagagctggatggagtccgaatttcgcgtctactcaagcgccaataattgcacttttgagtatgtgtcccagccctttttgatggatctggagggaaagcagggcaatttcaaaaatctgagagaattcgtttttaagaatatagatggatacttcaaaatctacagcaaacacacacccataaatcttgtgcgcgatcttccccagggcttcagcgcgttggaaccccttgttgacttgcccataggcatcaacattaccaggttccaaacgctgctcgccctccaccgcagctacttgacacccggggattccagctccggatggaccgccggcgccgcagcgtattatgtggggtacctgcaacccaggacatttttgctcaagtacaatgagaatgggaccatcacagatgcggtagactgtgcactggatccactcagcgaaactaaatgtaccctgaaaagctttaccgtggagaaaggaatctaccaaaccagcaacttcagggtccagcccactgaatccatcgttagatttccaaatataactaatttgtgtccatttggagaggtgttcaatgctacaaggttcgcgtctgtatacgcttggaaccggaagcgcatctcaaattgcgtggctgattatagcgttctttacaacagcgcttccttttccacgttcaagtgctatggtgtatccccgacaaagctgaatgacttgtgcttcaccaatgtgtatgcggattctttcgttattcgaggcgatgaagtcagacaaattgcgcctggccagaccggaaagattgccgactacaactataaactgccggacgactttactggttgcgtgatcgcttggaacagcaataatcttgatagtaaagttggaggaaactacaattacctctatagactgttcagaaagagcaacttgaagccattcgaacgggatatctctacggagatctatcaagctggcagcaccccctgcaatggtgtggaaggctttaattgttattttcctttgcagagctatggcttccaacctaccaacggagtgggctaccagccctacagagtggtggtgctcagctttgaactgctgcatgccccggccacagtttgcgggcccaaaaaaagcacgaatctggttaagaacaaatgcgtcaacttcaattttaatgggttgacaggtacaggcgtactgaccgaatccaacaaaaagttcctgccttttcagcagttcgggagagatatcgccgacactacagacgccgtcagggatccccaaacactcgaaattctggacatcacaccttgttccttcggcggggtatctgtgattactccgggcacaaataccagtaaccaggtagcggtgctttaccaggatgtcaactgtacggaagtacctgtcgctattcatgcggatcaactcactcctacctggagagtttattccactgggtccaacgtgtttcagacccgagccggctgcttgattggcgcggaacatgttaacaactcctacgaatgtgacatccctatcggagctggcatctgtgcttcctatcaaacgcaaacgaacagcccatctgctgctggttccgtagcctctcaaagcatcatcgcttatactatgtccttgggggctgaaaacagcgttgcctattccaacaatagcatcgctatccctaccaactttaccatttccgtgaccacagaaatactgccggtgagcatgacaaagacttctgtggactgtaccatgtatatatgcggcgatagcacagagtgttctaatttgctgctgcagtacggcagcttttgtacccaactcaacagagcacttacagggattgccgtcgagcaggataaaaacacccaggaggttttcgcccaggttaagcagatctacaagaccccaccaatcaaggatttcggcggcttcaatttttcccagatactgcccgatccttccaagccatccaaaaggagctttatagaggatctgctgttcaacaaggtgactctggccgacgctggctttatcaagcaatatggcgattgcctgggggatattgccgctagggaccttatctgcgctcaaaaattcaacggtcttaccgttctcccgcccctgctcaccgacgagatgatagcccagtacacgagcgcacttttggccggcacgataaccagcggctggacattcggtgccggggccgctcttcaaatcccctttgccatgcagatggcctacagatttaatgggataggcgtgacacaaaatgtcttgtatgaaaatcagaaactgattgcaaaccagtttaatagcgctattggcaagatccaagatagcctttcctccaccgcatccgctctgggaaagttgcaagacgtcgtgaatcaaaacgcccaagctctgaataccctcgtgaagcagcttagctccaactttggcgcgatatcctccgtgctgaacgatatcctgtccagattggacaaggtcgaggcagaagtccagatcgatagattgataaccggcagactccagtctctgcagacatatgtgactcagcagttgataagagcggccgaaatacgagcgtctgcaaatctcgcagcaacgaaaatgtcagagtgtgtattggggcaaagtaaaagagtagatttctgtggaaagggttaccatctgatgtcattcccccagtctgcaccacatggagtagtttttttgcatgtgacttatgtgcctgcccaggagaaaaatttcaccactgcacctgcgatctgtcatgacggcaaggcacatttccctagagaaggcgtcttcgtatcaaatggaacacactggtttgtaacccaaaggaacttttacgagccccaaattataactaccgacaacaccttcgtaagcggaaactgcgacgtcgttatagggatagtcaataatacggtctatgaccctcttcagccggaactggactcctttaaagaagaactggataagtacttcaagaaccatacgtctccggatgtggatctcggagatataagtggaatcaacgcaagcgtagtaaacattcagaaggagatagaccgactcaatgaggttgctaaaaacctgaacgaaagcttgatagacttgcaggagctgggtaagtacgaacagtacattaagtggccatggtatatctggttgggcttcatagcaggactcatagctatcgtcatggtgacaataatgctttgttgtatgaccagctgttgttcttgtctgaaaggctgctgcagctgtggcagctgttgtaaatttgacgaagatgattccgagcctgtgcttaagggcgtaaaactccactatacatgagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaggtttagtgaaccgtcagatccgctagcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttatgctgctgctgcccttccagttgctggctgtcctctttcccggcggcaactccgaggattacaaggacgacgacgacaagggtggaggctctggaggtggctctggtggaggttccggtggcggatctatgagcgacaacggtccccagaatcaaagaaatgcgcccagaattacattcggcggcccttctgatagcactggctcaaatcaaaacggggagagaagcggagccaggtccaaacagcggagaccccaaggcctgcctaataacaccgcttcctggttcacagctctgacgcaacacggcaaggaggatctgaagtttccacggggtcagggcgtcccgattaacacgaactctagcccagatgaccaaatagggtactacagaagagcgacaaggcggatcagaggaggcgatggaaaaatgaaggatctgtcccctaggtggtatttctattacctgggcacaggccctgaagctgggttgccttacggcgcaaacaaagatggaattatatgggtggccaccgagggggcgttgaacaccccaaaggatcacatcggaacgaggaatcccgccaacaatgctgctatagtgctccaactgccacagggaacaaccctgcctaagggcttctacgccgaggggagccgcggtggcagccaggccagctccagaagttcctcccgcagccggaacagctctagaaacagcactcccggcagctccagagggacaagcccagccagaatggccggcaatggcggcgacgctgccctcgcacttctgttgcttgatcggctcaatcaactcgaaagcaaaatgtccggcaagggacaacaacagcaaggacagaccgttacaaaaaaaagcgccgccgaggctagcaagaagcccagacagaagcgaaccgcaacaaaggcctataatgtaacacaagcctttggaaggcggggacccgaacagacccagggaaattttggcgaccaggaactgatccggcaagggacagactataaacattggccacagatagcgcaatttgctccctccgcctccgccttctttggcatgtcaagaataggcatggaagtaactccttctggaacctggctgacgtacactggggcaatcaagttggatgataaggaccctaatttcaaggaccaagttattttgctcaacaagcatatagacgcctacaagactttcccgcctaccgaacctaaaaaggataagaagaagaaagcagacgagacccaggccctgcctcaacggcaaaagaagcagcaaactgtgacactcctgcccgccgctgacttggatgatttttcaaaacagctccaacagagtatgagcagcgccgatagcacccaagctggaccgggtccgggcaacctggtgccgatggtggcgaccgtgggtccaggaccgggtatgctgatccccatcgccgtgggcggggccctggccggcctcgtgctgatcgtccttatcgcctacctcatcggcaagaagcactgctcatatcaggacatcctgtgagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggta

SEQ ID NO: 12 - protein, 1285 aa, synthetic construct
  REGION 1..1285: note = SARS-CoV2 spike mutant (p68)
MLLLPFQLLAVLFPGGNSEYPYDVPDYAGGGSGGGSGGGSGGGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSAAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKKHCSYQDIL

SEQ ID NO: 13 - DNA, 40827 bp, synthetic construct
  misc_feature 1..40827: note = p68 vector sequence
  source 1..40827: mol_type = other DNA
tattattgatgatgttaattaacatgcatggatcctacgtctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggatcgaattaattcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttatgcttctgcttcctttccaattgctggctgttctctttcctggaggtaactctgaatatccgtatgatgtgccggattatgcgggtggaggctctggaggtggctctggtggaggttccggtggcggatctcaatgtgtcaacctcaccacaaggacacagctccctcccgcatatacgaatagctttaccagaggcgtatactatcctgataaggtctttaggagctcagtactgcatagcactcaggatctcttcctgccgttcttcagtaatgttacttggtttcacgccattcatgtttccgggaccaatggcaccaaacggttcgataatccagtgcttcccttcaacgatggggtgtactttgccagcactgaaaaatctaatataattcggggatggattttcggaaccacactcgattccaagactcagtccctcttgatcgttaacaacgctactaatgttgtcattaaggtgtgtgagtttcagttctgcaacgaccctttcctgggtgtctactaccataaaaataacaagagctggatggagtccgaatttcgcgtctactcaagcgccaataattgcacttttgagtatgtgtcccagccctttttgatggatctggagggaaagcagggcaatttcaaaaatctgagagaattcgtttttaagaatatagatggatacttcaaaatctacagcaaacacacacccataaatcttgtgcgcgatcttccccagggcttcagcgcgttggaaccccttgttgacttgcccataggcatcaacattaccaggttccaaacgctgctcgccctccaccgcagctacttgacacccggggattccagctccggatggaccgccggcgccgcagcgtattatgtggggtacctgcaacccaggacatttttgctcaagtacaatgagaatgggaccatcacagatgcggtagactgtgcactggatccactcagcgaaactaaatgtaccctgaaaagctttaccgtggagaaaggaatctaccaaaccagcaacttcagggtccagcccactgaatccatcgttagatttccaaatataactaatttgtgtccatttggagaggtgttcaatgctacaaggttcgcgtctgtatacgcttggaaccggaagcgcatctcaaattgcgtggctgattatagcgttctttacaacagcgcttccttttccacgttcaagtgctatggtgtatccccgacaaagctgaatgacttgtgcttcaccaatgtgtatgcggattctttcgttattcgaggcgatgaagtcagacaaattgcgcctggccagaccggaaagattgccgactacaactataaactgccggacgactttactggttgcgtgatcgcttggaacagcaataatcttgatagtaaagttggaggaaactacaattacctctatagactgttcagaaagagcaacttgaagccattcgaacgggatatctctacggagatctatcaagctggcagcaccccctgcaatggtgtggaaggctttaattgttattttcctttgcagagctatggcttccaacctaccaacggagtgggctaccagccctacagagtggtggtgctcagctttgaactgctgcatgccccggccacagtttgcgggcccaaaaaaagcacgaatctggttaagaacaaatgcgtcaacttcaattttaatgggttgacaggtacaggcgtactgaccgaatccaacaaaaagttcctgccttttcagcagttcgggagagatatcgccgacactacagacgccgtcagggatccccaaacactcgaaattctggacatcacaccttgttccttcggcggggtatctgtgattactccgggcacaaataccagtaaccaggtagcggtgctttaccaggatgtcaactgtacggaagtacctgtcgctattcatgcggatcaactcactcctacctggagagtttattccactgggtccaacgtgtttcagacccgagccggctgcttgattggcgcggaacatgttaacaactcctacgaatgtgacatccctatcggagctggcatctgtgcttcctatcaaacgcaaacgaacagcccatctgctgctggttccgtagcctctcaaagcatcatcgcttatactatgtccttgggggctgaaaacagcgttgcctattccaacaatagcatcgctatccctaccaactttaccatttccgtgaccacagaaatactgccggtgagcatgacaaagacttctgtggactgtaccatgtatatatgcggcgatagcacagagtgttctaatttgctgctgcagtacggcagcttttgtacccaactcaacagagcacttacagggattgccgtcgagcaggataaaaacacccaggaggttttcgcccaggttaagcagatctacaagaccccaccaatcaaggatttcggcggcttcaatttttcccagatactgcccgatccttccaagccatccaaaaggagctttatagaggatctgctgttcaacaaggtgactctggccgacgctggctttatcaagcaatatggcgattgcctgggggatattgccgctagggaccttatctgcgctcaaaaattcaacggtcttaccgttctcccgcccctgctcaccgacgagatgatagcccagtacacgagcgcacttttggccggcacgataaccagcggctggacattcggtgccggggccgctcttcaaatcccctttgccatgcagatggcctacagatttaatgggataggcgtgacacaaaatgtcttgtatgaaaatcagaaactgattgcaaaccagtttaatagcgctattggcaagatccaagatagcctttcctccaccgcatccgctctgggaaagttgcaagacgtcgtgaatcaaaacgcccaagctctgaataccctcgtgaagcagcttagctccaactttggcgcgatatcctccgtgctgaacgatatcctgtccagattggacaaggtcgaggcagaagtccagatcgatagattgataaccggcagactccagtctctgcagacatatgtgactcagcagttgataagagcggccgaaatacgagcgtctgcaaatctcgcagcaacgaaaatgtcagagtgtgtattggggcaaagtaaaagagtagatttctgtggaaagggttaccatctgatgtcattcccccagtctgcaccacatggagtagtttttttgcatgtgacttatgtgcctgcccaggagaaaaatttcaccactgcacctgcgatctgtcatgacggcaaggcacatttccctagagaaggcgtcttcgtatcaaatggaacacactggtttgtaacccaaaggaacttttacgagccccaaattataactaccgacaacaccttcgtaagcggaaactgcgacgtcgttatagggatagtcaataatacggtctatgaccctcttcagccggaactggactcctttaaagaagaactggataagtacttcaagaaccatacgtctccggatgtggatctcggagatataagtggaatcaacgcaagcgtagtaaacattcagaaggagatagaccgactcaatgaggttgctaaaaacctgaacgaaagcttgatagacttgcaggagctgggtaagtacgaacagtacattaagtggccatggtatatctggttgggcttcatagcaggactcatagctatcgtcatggtgacaataatgctttgttgtatgaccagctgttgttcttgtctgaagaagcactgctcatatcaggacatcctgtgagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaggtttagtgaaccgtcagatccgctagcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttatgctgctgctgcccttccagttgctggctgtcctctttcccggcggcaactccgaggattacaaggacgacgacgacaagggtggaggctctggaggtggctctggtggaggttccggtggcggatctatgagcgacaacggtccccagaatcaaagaaatgcgcccagaattacattcggcggcccttctgatagcactggctcaaatcaaaacggggagagaagcggagccaggtccaaacagcggagaccccaaggcctgcctaataacaccgcttcctggttcacagctctgacgcaacacggcaaggaggatctgaagtttccacggggtcagggcgtcccgattaacacgaactctagcccagatgaccaaatagggtactacagaagagcgacaaggcggatcagaggaggcgatggaaaaatgaaggatctgtcccctaggtggtatttctattacctgggcacaggccctgaagctgggttgccttacggcgcaaacaaagatggaattatatgggtggccaccgagggggcgttgaacaccccaaaggatcacatcggaacgaggaatcccgccaacaatgctgctatagtgctccaactgccacagggaacaaccctgcctaagggcttctacgccgaggggagccgcggtggcagccaggccagctccagaagttcctcccgcagccggaacagctctagaaacagcactcccggcagctccagagggacaagcccagccagaatggccggcaatggcggcgacgctgccctcgcacttctgttgcttgatcggctcaatcaactcgaaagcaaaatgtccggcaagggacaacaacagcaaggacagaccgttacaaaaaaaagcgccgccgaggctagcaagaagcccagacagaagcgaaccgcaacaaaggcctataatgtaacacaagcctttggaaggcggggacccgaacagacccagggaaattttggcgaccaggaactgatccggcaagggacagactataaacattggccacagatagcgcaatttgctccctccgcctccgccttctttggcatgtcaagaataggcatggaagtaactccttctggaacctggctgacgtacactggggcaatcaagttggatgataaggaccctaatttcaaggaccaagttattttgctcaacaagcatatagacgcctacaagactttcccgcctaccgaacctaaaaaggataagaagaagaaagcagacgagacccaggccctgcctcaacggcaaaagaagcagcaaactgtgacactcctgcccgccgctgacttggatgatttttcaaaacagctccaacagagtatgagcagcgccgatagcacccaagctggaccgggtccgggcaacctggtgccgatggtggcgaccgtgggtccaggaccgggtatgctgatccccatcgccgtgggcggggccctggccggcctcgtgctgatcgtccttatcgcctacctcatcggcaagaagcactgctcatatcaggacatcctgtgagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggta

(19) United States
(12) Patent Application Publication
(10) Pub. No.: US 2025/0235530 A1
(43) Pub. Date: Jul. 24, 2025

(54) Anti COVID-19 Therapies targeting nucleocapsid and spike proteins

(71) Applicant: ImmunityBio, Inc., San Diego, CA (US)

(72) Inventors: Patrick Soon-Shiong, Culver City, CA (US); Peter Sieling, Culver City, CA (US); Kayvan Niazi, Culver City, CA (US); Shahrooz Rabizadeh, Agoura Hills, CA (US); Lise Geissert, Culver City, CA (US); Annie Shin, Culver City, CA (US); Adrian Rice, Culver City, CA (US); Elizabeth Gabitzsch, Culver City, CA (US); Jeffrey Safrit, Culver City, CA (US); Leonard Sender, Culver City, CA (US)

(73) Assignee: ImmunityBio, Inc., San Diego, CA (US)

(21) Appl. No.: 19/080,620

(22) Filed: Mar. 14, 2025

Related U.S. Application Data
(63) Continuation of application No. 18/488,629, filed on Oct. 17, 2023, now Pat. No. 12,290,558.
Application No. 18/488,629 is a continuation of application No. 17/198,164, filed on Mar. 10, 2021, now Pat. No. 11,857,620.
Application No. 17/198,164 is a continuation-in-part of application No. 17/082,994, filed on Oct. 28, 2020, now abandoned.
Application No. 17/082,994 is a continuation-in-part of application No. 16/883,263, filed on May 26, 2020, now Pat. No. 11,684,668.
Application No. 17/198,164 is a continuation-in-part of application No. 16/880,804, filed on May 21, 2020, now abandoned.
(60) Provisional application No. 63/135,380, filed on Jan. 8, 2021.
(60) Provisional application No. 63/121,102, filed on Dec. 3, 2020.
(60) Provisional application No. 63/022,146, filed on May 8, 2020.
(60) Provisional application No. 63/118,697, filed on Nov. 26, 2020.
(60) Provisional application No. 63/117,922, filed on Nov. 24, 2020.
(60) Provisional application No. 63/117,460, filed on Nov. 24, 2020.
(60) Provisional application No. 63/117,847, filed on Nov. 24, 2020.
(60) Provisional application No. 63/115,127, filed on Nov. 18, 2020.
(60) Provisional application No. 62/991,504, filed on Mar. 18, 2020.
(60) Provisional application No. 62/988,328, filed on Mar. 11, 2020.
(60) Provisional application No. 63/082,145, filed on Sep. 23, 2020.
(60) Provisional application No. 63/080,887, filed on Sep. 21, 2020.
(60) Provisional application No. 63/069,598, filed on Aug. 24, 2020.
(60) Provisional application No. 63/067,033, filed on Aug. 18, 2020.
(60) Provisional application No. 63/064,157, filed on Aug. 11, 2020.
(60) Provisional application No. 63/059,975, filed on Aug. 1, 2020.
(60) Provisional application No. 63/053,691, filed on Jul. 19, 2020.
(60) Provisional application No. 63/036,445, filed on Jun. 9, 2020.
(60) Provisional application No. 63/022,146, filed on May 8, 2020.
(60) Provisional application No. 63/016,048, filed on Apr. 27, 2020.
(60) Provisional application No. 63/016,241, filed on Apr. 27, 2020.
(60) Provisional application No. 63/009,960, filed on Apr. 14, 2020.
(60) Provisional application No. 63/010,010, filed on Apr. 14, 2020.
(60) Provisional application No. 62/991,504, filed on Mar. 18, 2020.
(60) Provisional application No. 62/988,328, filed on Mar. 11, 2020.
(60) Provisional application No. 63/022,146, filed on May 8, 2020.
(60) Provisional application No. 63/016,048, filed on Apr. 27, 2020.
(60) Provisional application No. 63/016,241, filed on Apr. 27, 2020.
(60) Provisional application No. 63/009,960, filed on Apr. 14, 2020.
(60) Provisional application No. 63/010,010, filed on Apr. 14, 2020.
(60) Provisional application No. 62/991,504, filed on Mar. 18, 2020.
(60) Provisional application No. 62/988,328, filed on Mar. 11, 2020.

(51) Int. Cl. A61K 39/215; A61K 39/00; A61K 39/39; A61P 31/14; C12N 15/86; C12N 15/861

(52) U.S. Cl. CPC A61K 39/215; C12N 15/861; A61K 39/39; A61K 2039/545; A61K 2039/55505; A61P 31/14; C07K 2319/06; C12N 15/86; C12N 2710/10343; C12N 2770/20022; C12N 2770/20034

(57) ABSTRACT

Disclosed herein are methods for inducing immunity against a virus such as a coronavirus in the mucosal tissue of a patient, include administering a vaccine composition to the patient by oral administration (e.g., nasal injection, nasal inhalation, oral inhalation, and/or oral ingestion). Also disclosed are compositions for assaying the presence of anti-viral antibodies induced by the administered vaccine or the presence of viral proteins in a saliva sample include a stabilizing solution and may also include the use of aragonite particle beads. Compositions and methods are presented for prevention and/or treatment of a coronavirus disease wherein the composition comprises comprises a recombinant entity. The recombinant entity is bivalent, comprising a nucleic acid encoding a coronavirus 2 nucleocapsid protein CoV2 nucleocapsid protein fused to an endosomal targeting sequence, and a nucleic acid encoding a CoV2 spike protein sequence optimized for cell surface expression.

This application is a CON of allowed Ser. No. 18/488,629 (filed Oct. 17, 2023), which is a CON of Ser. No. 17/198,164 (filed Mar. 10, 2021, now U.S. Pat. No. 11,857,620), which claims benefit of 63/121,102 (filed Dec. 3, 2020) and claims benefit of 63/117,847 (filed Nov. 24, 2020) and claims benefit of 63/115,127 (filed Nov. 18, 2020) and is a CIP of Ser. No. 17/082,994 (filed Oct. 28, 2020, now abandoned), which claims benefit of 62/991,504 (filed Mar. 18, 2020) and claims benefit of 62/988,328 (filed Mar. 11, 2020). This application claims benefit of 63/082,145 (filed Sep. 23, 2020) and claims benefit of 63/080,887 (filed Sep. 21, 2020) and claims benefit of 63/069,598 (filed Aug. 24, 2020) and claims benefit of 63/067,033 (filed Aug. 18, 2020) and claims benefit of 63/053,691 (filed Jul. 19, 2020) and claims benefit of 63/036,445 (filed Jun. 9, 2020) and is a CIP of Ser. No. 16/880,804 (filed May 21, 2020, now abandoned), which claims benefit of 63/022,146 (filed May 8, 2020) and claims benefit of 63/016,048 (filed Apr. 27, 2020) and claims benefit of 63/016,241 (filed Apr. 27, 2020) and claims benefit of 63/009,960 (filed Apr. 14, 2020) and claims benefit of 63/010,010 (filed Apr. 14, 2020) and claims benefit of 62/991,504 (filed Mar. 18, 2020) and claims benefit of 62/988,328 (filed Mar. 11, 2020). This application is a CIP of Ser. No. 16/883,263 (filed May 26, 2020, now U.S. Pat. No. 11,684,668), which claims benefit of 63/022,146 (filed May 8, 2020) and claims benefit of 63/016,048 (filed Apr. 27, 2020) and claims benefit of 63/016,241 (filed Apr. 27, 2020) and claims benefit of 63/009,960 (filed Apr. 14, 2020) and claims benefit of 63/010,010 (filed Apr. 14, 2020) and claims benefit of 62/991,504 (filed Mar. 18, 2020) and claims benefit of 62/988,328 (filed Mar. 11, 2020). This application claims benefit of 63/117,922 (filed Nov. 24, 2020) and claims benefit of 63/117,460 (filed Nov. 24, 2020) and claims benefit of 63/064,157 (filed Aug. 11, 2020) and claims benefit of 63/059,975 (filed Aug. 1, 2020) and claims benefit of 63/135,380 (filed Jan. 8, 2021) and claims benefit of 63/118,697 (filed Nov. 26, 2020). Each of the above applications are incorporated by reference in its entirety, including the drawings and the sequence listings.

### INCORPORATION OF SEQUENCE LISTING

This application contains references to nucleic acid and polypeptide sequences which have been submitted concurrently herewith as the sequence listing XML file “102538.0080US3-CON2”, created on Apr. 8, 2025. The file is 24,545 bytes in size. The aforementioned sequence listing is hereby incorporated by reference in its entirety pursuant to 37 C.F.R. § 1.52(e).

### FIELD

The present disclosure relates to composition and methods for administering a vaccine to a patient and monitoring induced immunity in the patient in a stabilized patient sample.

### BACKGROUND

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

After several noteworthy coronavirus outbreaks in the recent years, including SARS and MERS, Corona Virus Disease 2019 (COVID-19) is yet another example of a serious infectious disease precipitated by a member of the corona virus family. While diagnostic tests have become available in a relatively short time, testing is not efficient, and numerous attempts to treat the disease have so far not had significant success. Most typically, patients with severe symptoms are treated to maintain respiration/blood oxygenation, and supportive treatment is provided to reduce or prevent multi-organ damage or even failure. Despite such interventions, the mortality rate is significant, particularly in elderly, immune compromised individuals, and individuals with heart disease, lung disease, or diabetes.

Thus, even though various methods of addressing symptoms in patients with COVID-19 are known in the art, all or almost all of them suffer from various disadvantages. Consequently, there is a need to provide improved vaccine compositions and methods that render a therapeutic effect, reduce or prevent viral entry into a cell, reduce direct and indirect toxicity of the virus to the patient, and produce an immune response that is effective to clear the virus from the patient.

### SUMMARY

The present disclosure is directed to various immune therapeutic compositions and methods suitable for treating and/or preventing a coronavirus disease. In one aspect, disclosed herein is a replication defective adenovirus, wherein the adenovirus comprises an E1 gene region deletion; an E2b gene region deletion; an E3 gene region deletion; a nucleic acid encoding a coronavirus 2 (CoV2) nucleocapsid protein CoV2 nucleocapsid protein fused to an endosomal targeting sequence (N-ETSD), and a nucleic acid encoding a CoV2 spike protein sequence optimized for cell surface expression (S-Fusion). In a second aspect of this disclosure, provided herein is a recombinant yeast comprising a nucleic acid encoding a coronavirus 2 (CoV2) nucleocapsid protein CoV2 nucleocapsid protein fused to an endosomal targeting sequence (N-ETSD), and a nucleic acid encoding a CoV2 spike protein sequence optimized for cell surface expression (S-Fusion). comprising a nucleic acid encoding a coronavirus 2 (CoV2) nucleocapsid protein CoV2 nucleocapsid protein fused to an endosomal targeting sequence (ETSD), and a nucleic acid encoding a CoV2 spike protein sequence optimized for cell surface expression (S-Fusion). Preferably, the recombinant yeast is Saccharomyces cerevisiae.

In one embodiment of each of the above two aspects, the CoV2 nucleocapsid protein has at least 85% identity to SEQ ID NO:1. It is further contemplated that the fusion protein contains a linker between the ETSD domain and the nucleocapsid protein. For example this linker may be a 16 amino acid linker having the sequence (GGGS)₄. In one embodiment, the fusion protein has at least 85% identity of SEQ ID NO:2. The CoV2 spike protein is contemplated to have at least 85% identity to SEQ ID NO:6. The nucleic acid encoding the CoV2 spike protein has at least 99% identity to SEQ ID NO:5 or SEQ ID NO:7.

In another embodiment of this disclosure, the adenoviruses and yeasts disclosed herein may further comprise a nucleic acid encoding a trafficking sequence, a co-stimulatory molecule, and/or an immune stimulatory cytokine. The co-stimulatory molecule is selected from the group consisting of CD80, CD86, CD30, CD40, CD30L, CD40L, ICOS-L, B7-H3, B7-H4, CD70, OX40L, 4-1BBL, GITR-L, TIM-3, TIM-4, CD48, CD58, TL1A, ICAM-1, and LFA3. The immune stimulatory cytokine may be selected from the group consisting of IL-2, IL-12, IL-15, nogapendekin alfa-imbakicept, IL-21, IPS1, and LMP1.

In yet another embodiment, disclosed herein is a vaccine composition comprising the adenovirus or yeast as disclosed above, and wherein the composition is formulated for injection. The vaccine composition may be used for inducing immunity against CoV2 in a patient in need thereof, by administering to the patient the vaccine composition

In another aspect, the method includes administering to the subject an immunotherapy composition comprising a recombinant entity, wherein the recombinant entity comprises a nucleic acid that encodes a nucleocapsid protein of coronavirus 2 (CoV2) and/or a spike protein of CoV2. In one embodiment, the nucleocapsid protein is ETSD.

Preferably, the nucleic acid that encodes a nucleocapsid protein of coronavirus 2 further encodes a trafficking sequence for the nucleocapsid protein. It is further contemplated that the recombinant entity may also comprise a sequence that encodes at least one of a co-stimulatory molecule and an immune stimulatory cytokine. The co-stimulatory molecule is selected from the group consisting of CD80, CD86, CD30, CD40, CD30L, CD40L, ICOS-L, B7-H3, B7-H4, CD70, OX40L, 4-1BBL, GITR-L, TIM-3, TIM-4, CD48, CD58, TL1A, ICAM-1, and LFA3. The immune stimulatory cytokine is selected from the group consisting of IL-2, IL-12, IL-15, IL-15 super agonist (N803), IL-21, IPS1, and LMP1. In some preferred embodiments, the immune stimulatory cytokine is IL-15 super agonist N803.

The immunotherapy compositions disclosed herein to be administered subcutaneously or intravenously.

The recombinant entity contemplated herein may be a recombinant virus, such as a recombinant adenovirus. The recombinant entity may also be a recombinant yeast, such as Saccharomyces cerevisiae.

In some preferred embodiments, the coronavirus disease is COVID-19.

In yet another aspect of the present disclosure, disclosed herein is a vaccine formulation comprising a recombinant entity, wherein the recombinant entity comprises a nucleic acid that encodes a nucleocapsid protein of coronavirus 2 (CoV2); and/or wherein the recombinant entity encodes a spike protein of CoV2. As discussed throughout, the recombinant entity is preferably a recombinant adenovirus or Saccharomyces cerevisiae. The vaccine formulation may administered to a patient having a coronavirus disease for treatment and/or prevention of the coronavirus disease.

The present disclosure further provides methods and compositions for administering, monitoring, and assaying a vaccine. The contemplated methods include inducing immunity against a virus in a patient, administering a vaccine composition to the patient by administering a vaccine composition to the patient by delivery to the nasal mucosa, oral mucosa, and/or alimentary mucosa of the patient. Preferably, the vaccine targets severe acute respiratory syndrome (SARS)-like coronavirus (SARS-CoV2). The oral vaccine compositions described herein can serve as a booster vaccination to any initial prime vaccination against SARS-CoV2 S or N protein.

Notably, the disclosed methods also include obtaining a sample of saliva from the patient at a period of time after administering the vaccine. Typically, the sample of saliva is preserved in a stabilizing solution comprising glutaraldehyde, sodium benzoate, citric acid, propyl gallate, EDTA, zinc, actin, chitosan, parabens, sodium azide, or any combination thereof. More typically, the stabilizing solution comprises glutaraldehyde at 0.10 to 2.0% weight per volume (w/v), sodium benzoate at 0.10 to 1.0% w/v, and/or citric acid at 0.025 to 0.20% w/v. Additional embodiments include analyzing the sample of saliva for at least one selected from antibodies targeting the virus or a protein specific to the virus, wherein in the absence of antibodies in the sample saliva, the method further comprises administering a booster of the vaccine to the patient.

The oral vaccine compositions described herein can be used as a universal booster vaccine to any anti-SARS-CoV2 vaccine directed against the SARS-CoV2 spike (S) and/or nucleocapsid (N) proteins. This booster can work even in patients who were immunized with an anti-S or anti-N vaccine other than those described herein. In particular embodiments, the initial prime vaccine can be a lipid nanoparticle vaccine containing mRNA encoding the S protein, such as those vaccines currently being tested by Moderna and by Pfizer. In certain embodiments, the boost described herein is administered at least 7 days after the initial prime vaccination, for example at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 28 days, at least 35 days, or at least 42 days. The boost as described herein can effectively improve both antibody production against SARS-CoV2 and cell-mediate immunity against SARS-CoV2. The efficacy of the booster vaccine can be measured by any standard quantification of immune response (e.g., a QuantiFERON assay).

Additionally, the stabilizing solution further comprises aragonite particle beads having an average particle size of between 100 nm to 1 mm. The aragonite particle beads are capable of binding to immunoglobulin (Ig) proteins, anti-SARS-CoV2 antibodies, or a SAR-CoV2 viral protein. In exemplary embodiments, the aragonite particle beads are coupled to a recombinant ACE2 protein or a recombinant ACE2 alpha helix protein.

The contemplated subject matter also includes an aragonite composition formulated for binding an immunoglobulin (Ig) protein, an anti-SARS-CoV2 antibody protein, or a SARS-CoV2 viral protein. The aragonite composition includes a plurality of aragonite particle beads having an average particle size of between 100 nm to 1 mm, wherein the plurality of aragonite particle beads are functionalized with a moiety capable of binding to an immunoglobulin (Ig) protein, the anti-SARS-CoV2 antibody protein and/or the SARS-CoV2 viral protein.

In specific embodiments, the plurality of aragonite particle beads are functionalized with a moiety capable of binding to the anti-SARS-CoV2 comprises a recombinant ACE2 protein. For example, the moiety capable of binding to the anti-SARS-CoV2 may be selected from a recombinant ACE2 protein having at least 85% sequence identity to SEQ ID NO:1, a recombinant alpha-helix ACE2 protein of SEQ ID NO: 2, or the recombinant alpha-helix ACE2 protein having at least one mutation selected from T27F, T27W, T27Y, D30E, H34E, H34F, H34K, H34M, H34W, H34Y, D38E, D38M, D38W, Q24L, D30L, H34A, and/D355L.

Various objects, features, aspects, and advantages will become more apparent from the following detailed description of preferred embodiments, along with the accompanying drawing in which like numerals represent like components.

### BRIEF DESCRIPTION OF THE DRAWING

FIG. 1 exemplarily depicts vaccine constructs for Phase 1b clinical trials.

FIG. 2 exemplarily depicts in vitro Expression, Construct Expression via Western Blot, and detection of spike and nucleocapsid expression in by Western Blot.

FIG. 3 exemplarily depicts COVID-19 vaccine constructs.

FIG. 4 exemplarily depicts antibody response to N with a Th1 phenotype. Humoral Immune Responses T_{H}1 vs T_{H}2 associated isotype analysis is shown.

FIG. 5 exemplarily depicts cell mediated immunity (CMI) response to N focus phenotype—IFN-γ and IL-2 ELISpot.

FIG. 6 exemplarily depicts enhanced cell surface expression of RBD with S Fusion and with S Fusion+N combination constructs compared to S-WT.

FIG. 7 exemplarily depicts that recovered COVID-19 patient plasma recognizes antigens expressed by NANT's RBD-ETSD and NANT fusion S/N-ETSD constructs.

FIG. 8 exemplarily depicts the SARS-CoV-2 virus, spike, the hAd5 [E1-, E2b-, E3-] vector and vaccine candidate constructs. (a) Trimeric spike (S) protein (orange triangle) is displayed on the viral surface; the nucleocapsid (N) protein (blue circle) is associated with the viral RNA. (b) The Receptor Binding Domain (RBD) is within the S1 region, followed by other functional regions, the transmembrane domain (TM) and the C-terminus (CT), which is within the virus. (c) The second-generation human adenovirus serotype 5 (hAd5) vector used has the E1, E2b, and E3 regions deleted. Constructs are shown for (d) S wild type (S-WT), (e) S-RBD with the Enhanced T-cell Stimulation Domain (S RBD-ETSD), (f) S-Fusion, (g) N-ETSD, and (h) bivalent hAd5 S-Fusion+N-ETSD; LP—Leader peptide

FIG. 9 exemplarily depicts transfection of HEK293T cells with hAd5 S-Fusion+ETSD results in enhanced surface expression of the spike receptor binding domain (RBD). Flow cytometric analysis of an anti-RBD antibody with construct-transfected cells reveals no detectable surface expression of RBD in either S-WT or (b) S-WT+N-ETSD transfected cells. Surface RBD expression was high for S RBD-ETSD and S RBD-ETSD+N-ETSD (c, d). Expression was low in (e) S-Fusion transfected cells. Cell surface expression of the RBD was high in (f) S-Fusion+N-ETSD transfected cells, particularly at day 1 and 2. (g) No expression was detected the N-ETSD negative control. Y-axis scale is normalized to mode (NM).

FIG. 10 exemplarily depicts immunoblot analysis of S expression. Cell surface RBD expression with (a) hAd5 S-WT, S-Fusion, and (c) S-Fusion+N-ETSD in HEK 293T cells shows high correlation with (d) expression of S in immunoblots of HEK 293T cell lysates probed using anti-full length (S2) antibody. Y-axis scale is normalized to mode (NM).

FIG. 11 exemplarily depicts binding of recombinant ACE2-Fc HEK293T cell-surface expressed RBD after transfection confirms native protein folding. Flow cytometric analysis of binding between recombinant ACE2-Fc, with which the spike RBD interacts in vivo to initiate infection, and cell-surface antigens expressed after transfection of HEK293T cells with (a) hAd5 S-WT, (b) hAd5 S-Fusion, (c) hAd5 S-Fusion+N-ETSD, (d) hAd5 S RBD-ETSD, or (e) hAd5 S RBD-ETSD+N-ETSD constructs reveals the highest binding is seen for both ACE-Fc and an anti-RBD specific antibody (f-j) after transfection with the bivalent S-Fusion+N-ETSD. Both S RBD-ETSD-containing constructs also showed binding. Y-axis scale is normalized to mode (NM).

FIG. 12 exemplarily depicts N expressed from hAd5 N-ETSD is localized to the endosomal/lysosomal compartment. In HeLa cells infected with N-ETSD, (a) N (red) co-localizes with the endosomal marker CD71 (b) as indicated by the arrow in (c). In transfected HeLa cells, (d) N-ETSD also co-localizes with the lysosomal marker Lamp1, whereas (e) N wild type (N-WT) does not, showing instead diffuse cytoplasmic distribution.

FIG. 13 exemplarily depicts ICS detection of cytokine-expressing splenocytes from hAd5 S-Fusion+N-ETSD inoculated Day 28 CD-1 mice in response to peptide pools. (a) The highest CD8β⁺ splenocyte IFN-γ response was in hAd5 S-Fusion+N-ETSD-inoculated mouse splenocytes exposed to S peptide pool 1 (S-pep pool 1); splenocytes from these mice also expressed IFN-γ in response to the N peptide pool (N-pep pool). (b) CD4+ splenocytes from hAd5 S-Fusion+N-ETSD-inoculated mice only expressed IFN-γ in response to the N peptide pool. (c) IFN-γTNF-α responses of CD8f splenocytes from hAd5 S-Fusion+N-ETSD-inoculated mice were very similar to those in (a); as were (d) CD4+ splenocytes to the N peptide pool to those in (b). N=5 mice per group. All data sets graphed as the mean with SEM and all statistics performed using the Mann-Whitney test where *<0.05, **<0.01, ***<0.001, and ****<0.0001.

FIG. 14 exemplarily depicts anti-spike and anti-nucleocapsid antibody responses in sera from hAd5 S-Fusion+N-ETSD vaccinated mice. Based on absorbance, there was significant production of both (a) anti-S antibodies and (c) anti-nucleocapsid antibodies. (b, d) The ng equivalents are shown. Sera diluted 1:30 for anti-spike and 1:90 for anti-nucleocapsid antibodies. Data graphed as the mean and SEM. Statistical analysis was performed using an unpaired two-tailed Student's t-test where *<0.05, **<0.01, ***<0.001, and ****<0.0001.

FIG. 15 exemplarily depicts cPass and Vero E6 cell SARS-CoV-2 confirm neutralization by antibodies. (a) In the cPass assay, inhibition of S RBD interaction with ACE2 was significant at both 1:20 and 1:60 dilutions of serum from hAd5 S-Fusion+N-ETSD vaccinated mice. (b) The results in the Vero E6 cell SARS-CoV-2 viral infection for mice that showed S-specific antibodies by ELISA also showed high neutralization for mice and very high neutralization for pooled sera (G4 pool, blue line) even compared to COVID-19 convalescent serum. G4 pool—mice with S-specific antibodies; M1, M2, M3, M4—mouse ID; +C—convalescent serum; and media—media only negative control.

FIG. 16 exemplarily depicts isotypes for anti-spike and anti-nucleocapsid antibodies. (a, c) IgG2a and IgG2b isotype anti-spike and anti-nucleocapsid antibodies were significantly increased for hAd5 S-Fusion+N-ETSD mice compared to hAd5 Null mice. (b, d) The ng equivalents for antibody isotypes are shown. Data graphed as the mean and SEM. Statistical analysis was performed using an unpaired two-tailed Student's t-test where *<0.05, **<0.01, ***<0.001, and ****<0.0001.

FIG. 17 exemplarily depicts ELISpot detection of secreted cytokines. (a) IFN-□ secretion by hAd5 S-Fusion+N-ETSD splenocytes was significantly higher than hAd5 Null in response to both S peptide pool 1 and the N peptide pool; but (b) TL-4 was only secreted with hAd5 S-Fusion+N-ETSD in response to the N peptide pool (one high outlier in hAd5 null removed). N=5 mice per group. All data sets graphed as the mean with SEM and all statistics performed using the Mann-Whitney test where *<0.05, **<0.01, ***<0.001, and ****<0.0001.

FIG. 18 exemplarily depicts ratios for T-cell and humoral responses reveal Th1 predominance. (a) The ratio of total Th1 (IFN-γ) to Th2 (TL-4) spot-forming units is shown for responses to the combined S pools and to the N pool. (b) The Th1/Th2 ratio for antibodies against S and N is shown. For both (a) and (b) the dashed line indicates a ratio of 1 or a balance of Th1 and Th2 (no predominance).

FIG. 19 shows ELISA results detecting IgG seroreactivity against SARS-CoV2 spike in sera samples drawn from immunized macaques.

FIG. 20 breaks out the ELISA results in FIG. 1 for the Group 1 macaques.

FIG. 21 breaks out the ELISA results in FIG. 1 for the Group 1 macaques.

FIG. 22 illustrates one embodiment of the disclosure herein. (A) shows the ability of sera from vaccinated Group 1 macaques to inhibit SARS-CoV2 infectivity in vitro. (B) shows the ability of sera from vaccinated Group 2 macaques to inhibit SARS-CoV2 infectivity in vitro. The dotted line indicates 20% inhibition.

FIG. 23 illustrates one embodiment of the disclosure herein. (A) shows viral load (qPCR) in nasal swabs from Group 1 macaques. (B) shows viral load in nasal swabs from Group 2 macaques.

FIG. 24 illustrates one embodiment of the disclosure herein. (A) shows viral load (qPCR) in BAL from Group 1 macaques. (B) shows viral load in BAL from Group 2 macaques.

FIG. 25 shows ELISA results detecting IgG & IgM seroreactivity against SARS-CoV2 spike in sera samples drawn from human patients immunized with various experimental anti-SARS-CoV2 vaccines.

FIG. 26 shows Th1 ELISpot results from human patients 3, 6, & 11.

FIG. 27 shows Th1 ELISpot results from human patients 4 & 10.

FIG. 28 shows the results of a QuantiFERON assay on blood samples drawn from nine immunized patients on days 1 and 29 of the vaccination regime. The dashed line near the bottom of the panel indicates the threshold of detection. The dashed-line boxes indicate the results from subject #8.

### DETAILED DESCRIPTION

Disclosed herein are recombinant viruses and yeasts. The viruses and yeasts disclosed herein may be useful for a variety of purposes, such as treating and/or preventing a coronavirus disease. In one aspect, disclosed herein is a replication defective adenovirus, wherein the adenovirus comprises an E1 gene region deletion; an E2b gene region deletion; an E3 gene region deletion, a nucleic acid encoding a coronavirus 2 (CoV2) nucleocapsid protein CoV2 nucleocapsid protein fused to an endosomal targeting sequence (N-ETSD), and a nucleic acid encoding a CoV2 spike protein sequence optimized for cell surface expression (S-Fusion).

In one embodiment, the N-ETSD may comprises a sequence with at least 80% identity to SEQ ID NO:2. In other embodiments, the identity value is at least 85%. In still other embodiments, the identity value is at least 90%. In some embodiments, the identity value is at least 95%. In some embodiments, the identity value is at least 99%. In some embodiments, the identity value is 100%. It is further contemplated that the fusion protein contains a linker between the N-ETSD domain and the nucleocapsid protein. For example this linker may be a 16 amino acid linker having the sequence (GGGS)₄. In certain embodiments, methods are disclosed herein for enhancing the immunogenicity of an intracellular antigen, the methods comprising tagging the antigen with ETSD and expressing the tagged antigen in an antigen-presenting cell (e.g., a dendritic cell).

In some embodiments, the fusion protein comprising N-ETSD and CoV2 nucleocapsid protein may be encoded by a nucleic acid sequence having at least 80% identity to SEQ ID NO:3. In some embodiments, the identity value is at least 85%. In some embodiments, the identity value is at least 90%. In some embodiments, the identity value is at least 95%. In some embodiments, the identity value is at least 99%. In some embodiments, the identity value is 100%.

The CoV2 spike protein is contemplated to have at least 85% identity to SEQ ID NO:6. The nucleic acid encoding the CoV2 spike protein has at least 99% identity to SEQ ID NO:5 or SEQ ID NO:7.

In a second aspect of this disclosure, provided herein is a recombinant yeast comprising a nucleic acid encoding a protein selected from the group consisting of coronavirus 2 (CoV2) nucleocapsid protein, CoV2 spike protein, and a combination thereof. Preferably, the recombinant yeast is Saccharomyces cerevisiae.

In some embodiments of this second aspect, the CoV2 nucleocapsid protein comprises a sequence with at least 80% identity to SEQ ID NO:2 or SEQ ID NO:3. In other embodiments, the identity value is at least 85%. In still other embodiments, the identity value is at least 90%. In some embodiments, the identity value is at least 95%. In some embodiments, the identity value is at least 99%. In some embodiments, the identity value is 100%.

In some embodiment of this second aspect, the CoV2 spike protein comprises a sequence with at least 80% identity to SEQ ID NO:5. In other embodiments, the identity value is at least 85%. In still other embodiments, the identity value is at least 90%. In some embodiments, the identity value is at least 95%. In some embodiments, the identity value is at least 99%. In some embodiments, the identity value is 100%.

In some embodiments, the nucleic acid encoding the CoV2 spike protein comprises a sequence with at least 80% identity to SEQ ID NO:5 or SEQ ID NO:7. In other embodiments, the identity value is at least 85%. In still other embodiments, the identity value is at least 90%. In some embodiments, the identity value is at least 95%. In some embodiments, the identity value is at least 99%. In some embodiments, the identity value is 100%.

The adenoviruses and yeasts disclosed herein may further comprise a nucleic acid encoding a trafficking sequence, a co-stimulatory molecule, and/or an immune stimulatory cytokine. The co-stimulatory molecule is selected from the group consisting of CD80, CD86, CD30, CD40, CD30L, CD40L, ICOS-L, B7-H3, B7-H4, CD70, OX40L, 4-1BBL, GITR-L, TIM-3, TIM-4, CD48, CD58, TL1A, ICAM-1, and LFA3. The immune stimulatory cytokine may be selected from the group consisting of IL-2, IL-12, IL-15, nogapendekin alfa-imbakicept, IL-21, IPS1, and LMP1. Additionally or alternatively, the vaccines disclosed herein may also encode SARS-CoV-2 M protein, with or without an ETSD tag.

In yet another embodiment, disclosed herein is a vaccine composition comprising the adenovirus or yeast as disclosed above, and wherein the composition is formulated for injection. The vaccine composition may be used for inducing immunity against CoV2 in a patient in need thereof, by administering to the patient the vaccine composition

Also disclosed herein are methods for preventing and/or treating coronavirus diseases, and especially COVID-19. Preferably, the method includes using a viral or yeast vector that encodes the nucleocapsid protein and/or spike protein of the coronavirus in an immunogenic composition that is administered to a subject individual. The virus and/or yeast vaccine, thus administered, would infect the individual with CoV2 nucleocapsid or spike protein. With that in place, the individual would have an immune response against it, and be vaccinated. Notably, as the nucleocapsid protein and the spike protein are relatively conserved polypeptides, immune responses can be elicited for a variety of members of the coronavirus family.

Where the recombinant vector is an adenovirus, the adenoviral vector may be modified to encode the nucleocapsid protein, and/or the spike protein. Similarly, in case of yeast, the yeast vector may also be modified to encode the nucleocapsid protein, and/or the spike protein. Positive responses were obtained on cell mediated immunity upon administration of immunogenic compositions comprising the viral and/or yeast vectors in patients in need therof. Thus, in one embodiment, the present disclosure envision creating the coronaviral spikes to be expressed on the yeast surface. So, in this embodiment, the yeast is acting as an avatar coronavirus to stimulate the B cells. The stimulation of the B cells then results in humoral immunity.

In another embodiment, disclosed herein is a next generation bivalent human adenovirus serotype 5 (hAd5) vaccine capable of inducing immunity in patients with pre-existing adenovirus immunity, comprising both an S sequence optimized for cell surface expression (S-Fusion) and a conserved nucleocapsid (N) antigen designed to be transported to the endosomal subcellular compartment, with the potential to generate durable immune protection. As further described in this disclosure, this bivalent vaccine has been found to be is optimized for immunogenicity as evidenced by the following findings:
The optimized S-Fusion displayed improved S receptor binding domain (RBD) cell surface expression compared to S-WT where little surface expression was detected;
The expressed RBD from S-Fusion retained conformational integrity and recognition by ACE2-Fc;
The viral N protein modified with an enhanced T-cell stimulation domain (ETSD) localized to endosomal/lysosomal subcellular compartments for MHC I/II presentation; and
These optimizations to S and N (S-Fusion and N-ETSD) generated enhanced de novo antigen-specific B cell and CD4+ and CD8+ T-cell responses in antigen-naive pre-clinical models.

Both the T-cell and antibody immune responses to S and N demonstrated a T-helper 1 (Th1) bias. The antibody responses were neutralizing as demonstrated by two independent SARS-CoV-2 neutralization assays. Thus, in one embodiment, this next generation bivalent hAd5 S-Fusion+N-ETSD vaccine provides robust, durable cell-mediated and humoral immunity against SARS-CoV-2 infection. This vaccine construct may be administered orally, intranasaly or sublingually. Thus, in one embodiment, the instant disclosure provides vaccine construct in oral, intranasal, and sublingual formulations to induce mucosal immunity in addition to cell-mediated and humoral immunity. In one embodiment, the COVID-19 vaccine disclosed herein generates long-term T and B cell memory.

### Coronaviruses and Vaccines Therefor

Coronaviruses are found in avian and mammalian species. They resemble each other in morphology and chemical structure: for example, the coronaviruses of humans and cattle are antigenically related. There is no evidence, however, that human coronaviruses can be transmitted by animals. In animals, various coronaviruses invade many different tissues and cause a variety of diseases in humans. One such disease was Severe acute respiratory syndrome (SARS) coronavirus disease that spread to several countries in Asia, Europe and North America in late 2002/early 2003. Another such disease is the novel Coronvirus Disease of 2019 (COVID 19) that has spread to several countries in the world. In December of 2019, reports emerged from Wuhan, China concerning a new infectious respiratory disease with high morbidity and mortality1-3 that displayed human-to-human transmission.4 The causative agent was rapidly identified as a novel coronavirus and was designated SARS-coronavirus 2 (SARS-CoV-2). The disease it causes is referred to as COVID-19 and has rapidly become a worldwide pandemic that has disrupted socioeconomic life and resulted in more than 32 million infections and more than 1,100,000 deaths worldwide as of late October 2020.

COVID 19 usually begins with a fever greater than 38° C. Initial symptoms can also include cough, sore throat, malaise and mild respiratory symptoms. Within two days to a week, patients may have trouble breathing. Patients in more advanced stages of COVID 19 develop either pneumonia or respiratory distress syndrome. Public health interventions, such as surveillance, travel restrictions and quarantines, are being used to contain the spread of COVID 19. It is unknown, however, whether these draconian containment measures can be sustained with each appearance of the COVID 19 in humans. Furthermore, the potential of this new and sometimes lethal CoV as a bio-terrorism threat is obvious.

Coronavirus virions are spherical to pleomorphic enveloped particles. The envelope is studded with projecting glycoproteins, and surrounds a core consisting of matrix protein enclosed within which is a single strand of positive-sense RNA (Mr 6×10⁶) associated with nucleocapsid protein. In that regard, it should be noted that the terms “nucleocapsid protein,” “nucleoprotein,” and “nucleocapsid” are used interchangeably throughout this disclosure. The coronavirus nucleocapsid (N) is a structural protein found in all coronaviruses, including COVID 19. The nucleocapsid protein forms complexes with genomic RNA, interacts with the viral membrane protein during virion assembly and plays a critical role in enhancing the efficiency of virus transcription and assembly.

Another protein found throughout all coronavirus virions is the viral spike(S) protein. Coronaviruses are large positive-stranded RNA viruses typically with a broad host range. Like other enveloped viruses, CoV enter target cells by fusion between the viral and cellular membranes, and that process is mediated by the viral spike (S) protein.

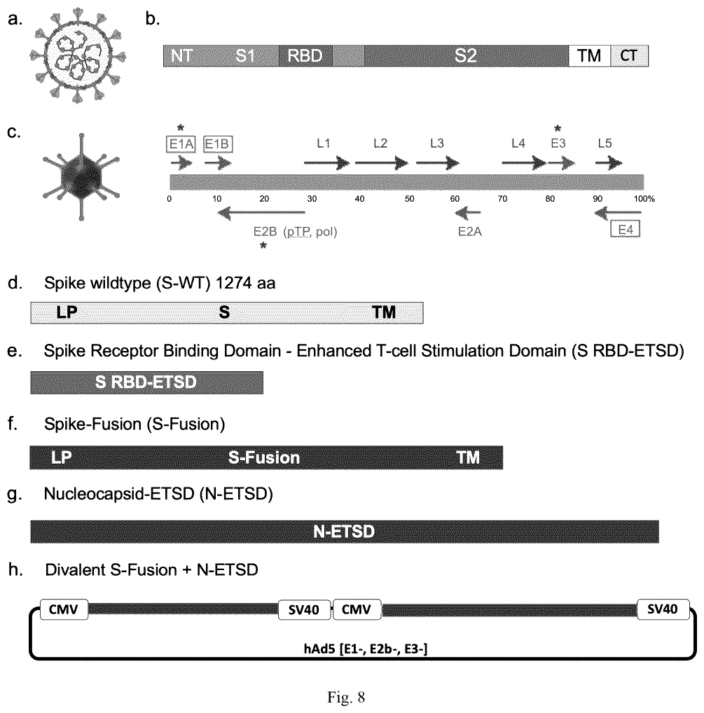

SARS-CoV-2 is an enveloped positive sense, single-strand RNA R coronavirus primarily composed of four structural proteins—spike (S), nucleocapsid (N), membrane (M), and envelope—as well as the viral membrane and genomic RNA. Of these, S is the largest and N the most prevalent. The S glycoprotein is displayed as a trimer on the viral surface (FIG. 8a), whereas N is located within the viral particle. A schematic of the S primary structure is shown in FIG. 8b. The sequence of SARS-CoV-2 was published8 and compared to that of previous coronaviruses. This was soon followed by reports on the crystal structure of the S protein. The virus uses S protein to enter host cells by interaction of the S receptor binding domain (S RBD) with angiotensin-converting enzyme 2 (ACE2), an enzyme expressed broadly on a variety of cell types in the nose, mouth, gut and lungs as well as other organs, and importantly on the alveolar epithelial cells of the lung where infection is predominantly manifested. As represented in FIG. 8b, the S RBD is found within the S1 region of spike.

The methods and compositions disclosed herein target the nucleoprotein and the spike protein that is conserved in all types of coronaviruses. In one embodiment, the present disclosure provides a vaccine formulation comprising a recombinant entity, wherein the recombinant entity comprises a nucleic acid that encodes a nucleocapsid protein of coronavirus 2 (CoV2); and/or wherein the recombinant entity encodes a spike protein of CoV2. The vaccine formulation may be useful for treating a disease, such as a coronavirus mediated disease or infection. Thus, in another embodiment, disclosed is a method for treating a coronavirus disease, in a patient in need thereof, comprising: administering to the subject an immunotherapy composition comprising a recombinant entity, wherein the recombinant entity comprises a nucleic acid that encodes a nucleocapsid protein of coronavirus 2 (CoV2). The coronavirus contemplated herein may be coronavirus disease 2019 (COVID-19) and/or severe acute respiratory syndrome coronavirus 2 (SARS-CoV2)

The instant disclosure also provides a method for treating coronavirus disease 2019 (COVID-19) and/or severe acute respiratory syndrome coronavirus 2 (SARS-CoV2), in a patient in need thereof, comprising: administering to the subject a first immunotherapy composition comprising a recombinant virus, wherein the recombinant virus comprises a nucleic acid that encodes a nucleocapsid protein of coronavirus 2 (CoV2), administering to the subject a second immunotherapy composition comprising a recombinant yeast, wherein the recombinant yeast comprises a nucleic acid that encodes a spike protein of CoV2. The first and second immunotherapy compositions may be administered concurrently or sequentially to the patient.

Viewed form a different perspective, contemplated herein is a viral vector (e.g., recombinant adenovirus genome, optionally with a deleted or non-functional E2b gene) that comprises a nucleic acid that encodes (a) at least a nucleocapsid protein; and (b) at least one spike protein. The viral vector may further comprise co-stimulatory molecule. Most typically, the nucleic acid will further include a trafficking signal to direct a peptide product encoded by the nucleic acid to the cytoplasm, the endosomal compartment, or the lysosomal compartment, and the peptide product will further comprise a sequence portion that enhances intracellular turnover of the peptide product.

The majority of current SARS-CoV-2 vaccines under development target S because of the potential to neutralize the ability of the virus to bind host cells by production of antibodies against the RBD. Support for RBD as a key antigen was recently confirmed, and it was reported that in 44 hospitalized COVID-19 patients, RBD-specific IgG responses and neutralizing antibody titers are detectable in all patients by 6 days post-PCR confirmation of infection, and that the two are correlated. See Suthar, M. S. et al. Rapid generation of neutralizing antibody responses in COVID-19 patients. Cell Reports Medicine, 2020, which is incorporated by reference herein. They confirmed this finding in an additional 231 PCR-confirmed COVID-19 patient samples. In addition to humoral responses, S epitopes are also frequent targets of COVID-19 recovered patient T cells, providing further justification for inclusion of S in prophylactic immunization strategies.

Despite the urgent need for rapid development of SARS-CoV-2 vaccines, reliance on any one antigen cargo or immunological pathway as occurring in the monovalent vaccines under development is not without risk. Evaluation of nearly 4000 SARS-CoV-2 genomic sequences has identified numerous mutations in S with the D614G variant emerging recently as a potentially more infectious strain six months after identification of the original virus.

In designing the vaccine disclosed herein, to overcome the risk of the emergence of new strains of the virus with mutations in S and to provide additional antigens against which responses can be elicited, an optimized N sequence was added. The N protein is a highly conserved and antigenic SARS-CoV-2-associated protein that has been studied previously as an antigen in coronavirus vaccine design for SARS-CoV. N associates with viral RNA within the virus and has a role in viral RNA replication, virus particle assembly, and release. SARS-CoV-2 N is a highly antigenic protein and recent studies have shown that nearly all patients infected with SARS-CoV-2 have antibody responses to N. Furthermore, another study reported that most, if not all, COVID-19 survivors tested were shown to have N-specific CD4+ T-cell responses.

Currently, there is keen focus on generation of humoral responses to vaccines with, arguably, less attention being paid to T-cell responses. The natural history of SARS-CoV-2 infection would suggest, however, that a robust T-cell response to vaccination is at least as important as the production of antibodies and should be a critical consideration for COVID-19 vaccine efficacy.

First, the humoral and T-cell responses are highly correlated, with titers of neutralizing antibodies being proportional to T-cell levels, suggesting the T response is necessary for an effective humoral response. It is well established that the activation of CD4+ T helper cells enhances B-cell production of antibodies. Second, virus-specific CD4+ and CD8+ T cells are not only widely detected in COVID-19 patients, based on findings from patients recovered from the closely-related SARS-CoV, but such T cells persist for at least 6-17 years, suggesting that T cells may be an important part of long-term immunity. These T-cell responses were predominantly to N, and it has been reported that in all 36 convalescent COVID-19 patients in their study, the presence of CD4+ and CD8+ T cells recognizing multiple regions of the N protein could be demonstrated. Examination of blood from 23 individuals who had recovered from SARS-CoV and found that the memory T cells acquired 17 years ago also recognized multiple proteins of SARS-CoV-2. These findings emphasize the importance of designing a vaccine with the highly conserved nucleocapsid present in both SARS-CoV and SARS-CoV-2. Third, recovered patients exposed to SARS-CoV-2 have been found without seroconversion, but with evidence of T-cell responses. The T-cell based responses become even more critical given the finding in at least one study that neutralizing antibody titers decline in some COVID-19 patients after about 3 months.

In one embodiment, the vaccines disclosed herein results in the generation of T-cell in addition to humoral responses. A bivalent vaccine comprising many antigens—S RBD as displayed by inclusion of full-length S including SD1, S1 and S2 epitopes, along with N—would be more effective in eliciting both T-cell and antibody-based responses than a construct with either antigen alone by presenting both unique and conserved SARS-CoV-2 antigenic sites to the immune system. The importance of both S and N was highlighted by identifying that both S and N antigens as a priori potential B and T-cell epitopes for the SARS-CoV virus that shows close similarity to SARS-CoV-2 that are predicted to induce both T and B cell responses.

An additional consideration for design of an effective vaccine is the likelihood of antigen presentation on the surface of the vectored-protein-expressing cell and in a conformation that recapitulates natural virus infection. First, because wild type N does not have a signaling domain that directs it to endosomal processing and ultimately MHC class II complex presentation to CD4+ T cells, the wild type N sequence is not optimal for induction of a vigorous CD4+ T-cell responses, a necessity for both cell-mediated and B cell memory. To overcome this limitation, we have designed an Enhanced T-cell Stimulation Domain (ETSD) to N to allow the necessary processing and presentation. Second, to display the highly antigenic RBD region of S on the cell surface, we have optimized the wild type S protein “S Fusion sequence”, to increase the likelihood of native folding, increased stability, and proper cell surface expression of RBD. Thus, in one embodiment, the vaccine construct design comprises an S-Fusion+N-ETSD sequence.

The vaccine platform utilized here is a next-generation recombinant human adenovirus serotype 5 (hAd5) vector with deletions in the E1, E2b, and E3 gene regions (hAd5 [E1-, E2b-, E3-]). This hAd5 [E1-, E2b-, E3-] vector (FIG. 8c) is primarily distinguished from other first-generation [E1-, E3-] recombinant Ad5 platforms by having additional deletions in the early gene 2b (E2b) region that remove the expression of the viral DNA polymerase (pol) and in pre terminal protein (pTP) genes, and its propagation in the E.C7 human cell line. Removal of these E2b regions confers advantageous immune properties by minimizing immune responses to Ad5 viral proteins such as viral fibers, 37 thereby eliciting potent immune responses to specific antigens in patients with pre-existing adenovirus (Ad) immunity. As a further benefit of these deletions, the vector has an expanded gene-carrying/cloning capacity compared to the first generation Ad5 [E1-, E3-] vectors. This next generation hAd5 [E1-, E2b-, E3-] vaccine platform, in contrast to Ad5 [E1-, E3-]-based platforms, does not promote activities that suppress innate immune signaling, thereby allowing for improved vaccine efficacy and a superior safety profile independent of previous Ad immunity. Since these deletions allow the hAd5 platform to be efficacious even in the presence of existing Ad immunity, this platform enables relatively long-term antigen expression without significant induction of anti-vector immunity. It is therefore also possible to use the same vector/construct for homologous prime-boost therapeutic regimens unlike first-generation Ad platforms which face the limitations of pre-existing and vaccine-induced Ad immunity. Importantly, this next generation Ad vector has demonstrated safety in over 125 patients with solid tumors. In these Phase I/II studies, CD4+ and CD8+ antigen-specific T cells were successfully generated to multiple somatic antigens (CEA, MUC1, brachyury) even in the presence of pre-existing Ad immunity.

The instant disclosure provides findings of confirmed enhanced cell-surface expression and physiologically-relevant folding of the expressed S RBD from S-Fusion by ACE2-Fc binding. The N-ETSD protein was successfully localized to the endosomal/lysosomal subcellular compartment for MHC presentation and consequently generated both CD4+ and CD8+ T-cell responses. Immunization of CD-1 mice with the hAd5 S Fusion+N-ETSD vaccine elicited both humoral and cell-mediated immune responses to vaccine antigens. CD8+ and CD4+ T-cell responses were noted for both S and N. Statistically significant IgG responses were seen for antibody generation against S and N. Potent neutralization of SARS-CoV-2 by sera from hAd5 S Fusion+N-ETSD-immunized mice was confirmed by two independent SARS-CoV-2 neutralization assays: the cPass assay measuring competitive inhibition of RBD binding to ACE2,44 and in the live SARS-CoV-2 virus assay with infected Vero E6 cells. Analysis of T-cell responses as well as humoral responses to S and N were skewed toward a Th1-specific response.

Taken together, these findings illustrate that hAd5 S-Fusion+N-ETSD vaccine would be particularly effective against the SARS-CoV-2.

### Recombinant Viruses

With respect to recombinant viruses it is contemplated that all known manners of making recombinant viruses are deemed suitable for use herein, however, especially preferred viruses are those already established in therapy, including adenoviruses, adeno-associated viruses, alphaviruses, herpes viruses, lentiviruses, etc. Among other appropriate choices, adenoviruses are particularly preferred.

Moreover, it is further generally preferred that the virus is a replication deficient and non-immunogenic virus. For example, suitable viruses include genetically modified alphaviruses, adenoviruses, adeno-associated viruses, herpes viruses, lentiviruses, etc. However, adenoviruses are particularly preferred. For example, genetically modified replication defective adenoviruses are preferred that are suitable not only for multiple vaccinations but also vaccinations in individuals with preexisting immunity to the adenovirus (see e.g., WO 2009/006479 and WO 2014/031178, which are incorporated by reference in its entirety). In some embodiments, the replication defective adenovirus vector comprises a replication defective adenovirus 5 vector. In some embodiments, the replication defective adenovirus vector comprises a deletion in the E2b region. In some embodiments, the replication defective adenovirus vector further comprises a deletion in the E1 region. In that regard, it should be noted that deletion of the E2b gene and other late proteins in the genetically modified replication defective adenovirus to reduce immunogenicity. Moreover, due to these specific deletions, such genetically modified viruses were replication deficient and allowed for relatively large recombinant cargo.

For example, WO 2014/031178 describes the use of such genetically modified viruses to express CEA (colorectal embryonic antigen) to provide an immune reaction against colon cancer. Moreover, relatively high titers of recombinant viruses can be achieved using genetically modified human 293 cells as has been reported (e.g., J Virol. 1998 February; 72(2): 926-933).

E1-deleted adenovirus vectors Ad5 [E1-] are constructed such that a trans gene replaces only the E1 region of genes. Typically, about 90% of the wild-type Ad5 genome is retained in the vector. Ad5 [E1-] vectors have a decreased ability to replicate and cannot produce infectious virus after infection of cells not expressing the Ad5 E1 genes. The recombinant Ad5 [E1-] vectors are propagated in human cells allowing for Ad5 [E1-] vector replication and packaging. Ad5 [E1-] vectors have a number of positive attributes; one of the most important is their relative ease for scale up and cGMP production. Currently, well over 220 human clinical trials utilize Ad5 [E1-] vectors, with more than two thousand subjects given the virus sc, im, or iv. Additionally, Ad5 vectors do not integrate; their genomes remain episomal. Generally, for vectors that do not integrate into the host genome, the risk for insertional mutagenesis and/or germ-line transmission is extremely low if at all. Conventional Ad5 [E1-] vectors have a carrying capacity that approaches 7 kb.

One obstacle to the use of first generation (E1-deleted) Ad5-based vectors is the high frequency of pre-existing anti-adeno virus type 5 neutralizing antibodies. Attempts to overcome this immunity is described in WO 2014/031178, which is incorporated by reference herein. Specifically, a novel recombinant Ad5 platform has been described with deletions in the early 1 (E1) gene region and additional deletions in the early 2b (E2b) gene region (Ad5 [E1-, E2b-]). Deletion of the E2b region (that encodes DNA polymerase and the pre-terminal protein) results in decreased viral DNA replication and late phase viral protein expression. E2b deleted adenovirus vectors provide an improved Ad-based vector that is safer, more effective, and more versatile than First Generation adenovirus vectors.

In a further embodiment, the adenovirus vectors contemplated for use in the present disclosure include adenovirus vectors that have a deletion in the E2b region of the Ad genome and, optionally, deletions in the E1, E3 and, also optionally, partial or complete removal of the E4 regions. In a further embodiment, the adenovirus vectors for use herein have the E1 and/or the preterminal protein functions of the E2b region deleted. In some cases, such vectors have no other deletions. In another embodiment, the adenovirus vectors for use herein have the E1, DNA polymerase and/or the preterminal protein functions deleted.

The term “E2b deleted”, as used herein, refers to a specific DNA sequence that is mutated in such a way so as to prevent expression and/or function of at least one E2b gene product. Thus, in certain embodiments, “E2b deleted” is used in relation to a specific DNA sequence that is deleted (removed) from the Ad genome. E2b deleted or “containing a deletion within the E2b region” refers to a deletion of at least one base pair within the E2b region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E2b region of the Ad genome. An E2b deletion may be a deletion that prevents expression and/or function of at least one E2b gene product and therefore, encompasses deletions within exons of encoding portions of E2b-specific proteins as well as deletions within promoter and leader sequences. In certain embodiments, an E2b deletion is a deletion that prevents expression and/or function of one or both of the DNA polymerase and the preterminal protein of the E2b region. In a further embodiment, “E2b deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As noted before, the desired nucleic acid sequences (for expression from virus infected cells) are under the control of appropriate regulatory elements well known in the art. In view of the above, it should be appreciated that compositions and methods presented are not only suitable for directing virally expressed antigens specifically to one or another (or both) MHC systems, but will also provide increased stimulatory effect on the CD8+ and/or CD4+ cells via inclusion of various co-stimulatory molecules (e.g., ICAM-1 (CD54), ICOS-L, LFA-3 (CD58), and at least one of B7.1 (CD80) and B7.2 (CD86)), and via secretion or membrane bound presentation of checkpoint inhibitors.

With respect to viral expression and vaccination systems it is contemplated that all therapeutic recombinant viral expression systems are deemed suitable for use herein so long as such viruses are capable to lead to expression of the recombinant payload in an infected cell.

Regardless of the type of recombinant virus it is contemplated that the virus may be used to infect patient (or non-patient) cells ex vivo or in vivo. For example, the virus may be injected subcutaneously or intravenously, or may be administered intranasaly or via inhalation to so infect the patient's cells, and especially antigen presenting cells. Alternatively, immune competent cells (e.g., NK cells, T cells, macrophages, dendritic cells, etc.) of the patient (or from an allogeneic source) may be infected in vitro and then transfused to the patient. Alternatively, immune therapy need not rely on a virus but may be effected with nucleic acid transfection or vaccination using RNA or DNA, or other recombinant vector that leads to the expression of the neoepitopes (e.g., as single peptides, tandem mini-gene, etc.) in desired cells, and especially immune competent cells.

As noted above, the desired nucleic acid sequences (for expression from virus infected cells) are under the control of appropriate regulatory elements well known in the art. For example, suitable promoter elements include constitutive strong promoters (e.g., SV40, CMV, UBC, EF1A, PGK, CAGG promoter), but inducible promoters are also deemed suitable for use herein, particularly where induction conditions are typical for a tumor microenvironment. For example, inducible promoters include those sensitive to hypoxia and promoters that are sensitive to TGF-β or IL-8 (e.g., via TRAF, INK, Erk, or other responsive elements promoter). In other examples, suitable inducible promoters include the tetracycline-inducible promoter, the myxovirus resistance 1 (Mx1) promoter, etc.

The replication defective adenovirus comprising an E1 gene region deletion, an E2b gene region deletion, and a nucleic acid encoding a coronavirus 2 (CoV2) nucleocapsid protein and/or a CoV2 spike protein, as disclosed herein may be administered to a patient in need for inducing immunity against CoV2. Routes and frequency of administration of the therapeutic compositions described herein, as well as dosage, may vary from individual to individual, and the severity of the disease, and may be readily established using standard techniques. In some embodiments, the administration comprises delivering 4.8-5.2×10¹¹ replication defective adenovirus particles, or 4.9-5.1×10¹¹ replication defective adenovirus particles, or 4.95-5.05×10¹¹ replication defective adenovirus particles, or 4.99-5.01×10¹¹ replication defective adenovirus particles.

The administration of the virus particles can be through a variety of suitable paths for delivery. One preferred route contemplated herein is by injection, such as intracutaneous injection, intramuscular injection, intravenous injection or subcutaneous injection. In some embodiments, a subcutaneous delivery may be preferred.

### Recombinant Yeasts

With respect to yeast expression and vaccination systems, it is contemplated that all known yeast strains are deemed suitable for use herein. However, it is preferred that the yeast is a recombinant Saccharomyces strain that is genetically modified with a nucleic acid construct encoding a protein selected from the group consisting of coronavirus 2 (CoV2) nucleocapsid protein, CoV2 spike protein, and a combination thereof, to thereby initiate an immune response against the CoV2 viral disease. In one aspect of any of the embodiments of the disclosure described above or elsewhere herein, the yeast vehicle is a whole yeast. The whole yeast, in one aspect is killed. In one aspect, the whole yeast is heat-inactivated. In one preferred embodiment, the yeast is a whole, heat-inactivated yeast from Saccharomyces cerevisiae.

The use of a yeast based therapeutic compositions are disclosed in the art. For example, WO 2012/109404 discloses yeast compositions for treatment of chronic hepatitis b infections.

It is noted that any yeast strain can be used to produce a yeast vehicle of the present disclosure. Yeasts are unicellular microorganisms that belong to one of three classes: Ascomycetes, Basidiomycetes and Fungi Imperfecti. One consideration for the selection of a type of yeast for use as an immune modulator is the pathogenicity of the yeast. In preferred embodiments, the yeast is a non-pathogenic strain such as Saccharomyces cerevisiae as non-pathogenic yeast strains minimize any adverse effects to the individual to whom the yeast vehicle is administered. However, pathogenic yeast may also be used if the pathogenicity of the yeast can be negated using pharmaceutical intervention.

For example, suitable genera of yeast strains include Saccharomyces, Candida, Cryptococcus, Hansenula, Kluyveromyces, Pichia, Rhodotorula, Schizosaccharomyces and Yarrowia. In one aspect, yeast genera are selected from Saccharomyces, Candida, Hansenula, Pichia or Schizosaccharomyces, and in a preferred aspect, Saccharomyces is used. Species of yeast strains that may be used include Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia lipolytica.

It should further be appreciated that a number of these species include a variety of subspecies, types, subtypes, etc. that are intended to be included within the aforementioned species. In one aspect, yeast species used in the instant disclosure include S. cerevisiae, C. albicans, H. polymorpha, P. pastoris and S. pombe. S. cerevisiae is useful due to it being relatively easy to manipulate and being “Generally Recognized As Safe” or “GRAS” for use as food additives (GRAS, FDA proposed Rule 62FR18938, Apr. 17, 1997). Therefore, particularly contemplated herein is a yeast strain that is capable of replicating plasmids to a particularly high copy number, such as a S. cerevisiae cir strain. The S. cerevisiae strain is one such strain that is capable of supporting expression vectors that allow one or more target antigen(s) and/or antigen fusion protein(s) and/or other proteins to be expressed at high levels. In addition, any mutant yeast strains can be used, including those that exhibit reduced post-translational modifications of expressed target antigens or other proteins, such as mutations in the enzymes that extend N-linked glycosylation.

Expression of contemplated peptides/proteins in yeast can be accomplished using techniques known to those skilled in the art. Most typically, a nucleic acid molecule encoding at least one protein is inserted into an expression vector such manner that the nucleic acid molecule is operatively linked to a transcription control sequence to be capable of effecting either constitutive or regulated expression of the nucleic acid molecule when transformed into a host yeast cell. As will be readily appreciated, nucleic acid molecules encoding one or more proteins can be on one or more expression vectors operatively linked to one or more expression control sequences. Particularly important expression control sequences are those which control transcription initiation, such as promoter and upstream activation sequences.

Any suitable yeast promoter can be used in the methods and compositions of the present disclosure and a variety of such promoters are known to those skilled in the art and have generally be discussed above. Promoters for expression in Saccharomyces cerevisiae include promoters of genes encoding the following yeast proteins: alcohol dehydrogenase I (ADH1) or II (ADH2), CUP1, phosphoglycerate kinase (PGK), triose phosphate isomerase (TPI), translational elongation factor EF-1 alpha (TEF2), glyceraldehyde-3-phosphate dehydrogenase (GAPDH; also referred to as TDH3, for triose phosphate dehydrogenase), galactokinase (GAL1), galactose-1-phosphate uridyl-transferase (GAL7), UDP-galactose epimerase (GAL10), cytochrome cl (CYC1), Sec7 protein (SEC7) and acid phosphatase (PHO5), including hybrid promoters such as ADH2/GAPDH and CYC1/GAL10 promoters, and including the ADH2/GAPDH promoter, which is induced when glucose concentrations in the cell are low (e.g., about 0.1 to about 0.2 percent), as well as the CUP1 promoter and the TEF2 promoter. Likewise, a number of upstream activation sequences (UASs), also referred to as enhancers, are known. Upstream activation sequences for expression in Saccharomyces cerevisiae include the UASs of genes encoding the following proteins: PCK1, TPI, TDH3, CYC1, ADH1, ADH2, SUC2, GAL1, GAL7 and GAL10, as well as other UASs activated by the GAL4 gene product, with the ADH2 UAS being used in one aspect. Since the ADH2 UAS is activated by the ADR1 gene product, it may be preferable to overexpress the ADR1 gene when a heterologous gene is operatively linked to the ADH2 UAS. Transcription termination sequences for expression in Saccharomyces cerevisiae include the termination sequences of the alpha-factor, GAPDH, and CYC1 genes. Transcription control sequences to express genes in methyltrophic yeast include the transcription control regions of the genes encoding alcohol oxidase and formate dehydrogenase.

Likewise, transfection of a nucleic acid molecule into a yeast cell according to the present disclosure can be accomplished by any method by which a nucleic acid molecule administered into the cell and includes diffusion, active transport, bath sonication, electroporation, microinjection, lipofection, adsorption, and protoplast fusion. Transfected nucleic acid molecules can be integrated into a yeast chromosome or maintained on extrachromosomal vectors using techniques known to those skilled in the art. As discussed above, yeast cytoplast, yeast ghost, and yeast membrane particles or cell wall preparations can also be produced recombinantly by transfecting intact yeast microorganisms or yeast spheroplasts with desired nucleic acid molecules, producing the antigen therein, and then further manipulating the microorganisms or spheroplasts using techniques known to those skilled in the art to produce cytoplast, ghost or subcellular yeast membrane extract or fractions thereof containing desired antigens or other proteins. Further exemplary yeast expression systems, methods, and conditions suitable for use herein are described in US20100196411A1, US2017/0246276, or US 2017/0224794, and US 2012/0107347.

So produced recombinant viruses and yeasts may then be individually or in combination used as a therapeutic vaccine in a pharmaceutical composition, typically formulated as a sterile injectable composition with a virus of between 10⁴-10¹³ virus or yeast particles per dosage unit, or more preferably between10⁹-10¹² virus or yeast particles per dosage unit. Alternatively, virus or yeast may be employed to infect patient cells ex vivo and the so infected cells are then transfused to the patient. However, alternative formulations are also deemed suitable for use herein, and all known routes and modes of administration are contemplated herein.

Second Generation hAd5 [E1-, E2b-, E3-] Based Vaccines Disclosed Herein Overcome Pre-Existing Anti-Ad5 Immunity

To avoid the Ad immunization barrier and circumvent the adverse conditions for first generation Ad5 [E1-E3-] vectors, an advanced 2nd generation human adenoviral (hAd5) vector was constructed having two (2) additional deletions in the E2b region, removing the DNA polymerase and the preterminal protein genes [E1-, E2b-, E3-]. (Former names of our adenovirus vector were Ad5, ETBX in literature)

E2b-deleted hAd5 vectors have up to a 12-14 kb gene-carrying capacity as compared to the 7-kb capacity of first generation Ad5 [E1-] vectors, providing space for multiple genes if needed. hAd5 [E1-, E2b-, E3-] based recombinant vectors are produced using the human E.C7 cell line. Deletion of the E2b region also confers advantageous immune properties on these novel Ad vectors, eliciting potent immune responses to specific, non-viral antigens while minimizing the immune responses to Ad viral proteins.

hAd5 [E1-, E2b-, E3-] vectors induce a potent cell mediated immune (CMI) response, as well as Abs against the vectored antigens even in the presence of Ad immunity. hAd5 [E1-, E2b-, E3-] vectors also have reduced adverse reactions as compared to Ad5 [E1-] vectors, in particular the appearance of hepatotoxicity and tissue damage. In one embodiment, the reduced inflammatory response against hAd5 [E1-, E2b-, E3-] vector viral proteins and the resulting evasion of pre-existing Ad immunity increases the capability for the hAd5 [E1-, E2b-, E3-] vectors to infect dendritic cells (DC), resulting in greater immunization of the vaccine. In addition, increased infection of other cell types provides high levels of antigen presentation needed for a potent CD8+ and CD4+ T cell responses, leading to memory T cell development. In one embodiment, hAd5 [E1-, E2b-, E3-] vectors are superior to Ad5 [E1-] vectors in immunogenicity and safety and will be the best platform to develop a COVID-19 vaccine in a rapid and efficient manner. In one embodiment, a prophylactic vaccine is tested against COVID-19 by taking advantage of this new hAd5 vector system that overcomes barriers found with other Ad5 systems and permits the immunization of people who have previously been exposed to Ad5.

Track Record of Rapid Vaccine Development Utilizing Second Generation Human (hAd5) Adenovirus Platform During Pandemic Treats: H1N1 Experience in 2009

To address emerging pathogen threats, especially in times of pandemic, it is critical that modernized vaccine technologies be deployed. These technologies will utilize the power of genomic sequencing, rapid transfection in well-established vaccine vectors to rapidly identify constructs with high immunogenicity.

Vaccines against emerging pathogens such as the 2009 H1N1 pandemic virus can benefit from current technologies such as rapid genomic sequencing to construct the most biologically relevant vaccine. A novel platform (hAd5 [E1-, E2b-, E3-]) has been utilized to induce immune responses to various antigenic targets. This vector platform expressed hemagglutinin (HA) and neuraminidase (NA) genes from 2009 H1N1 pandemic viruses. Inserts were consensuses sequences designed from viral isolate sequences and the vaccine was rapidly constructed and produced. Vaccination induced H1N1 immune responses in mice, which afforded protection from lethal virus challenge. In ferrets, vaccination protected from disease development and significantly reduced viral titers in nasal washes. H1N1 cell mediated immunity as well as antibody induction correlated with the prevention of disease symptoms and reduction of virus replication. The hAd5 [E1-, E2b-, E3-] has thus demonstrated the capability for the rapid development of effective vaccines against infectious diseases.

hAd5 Vaccine Constructs and Results

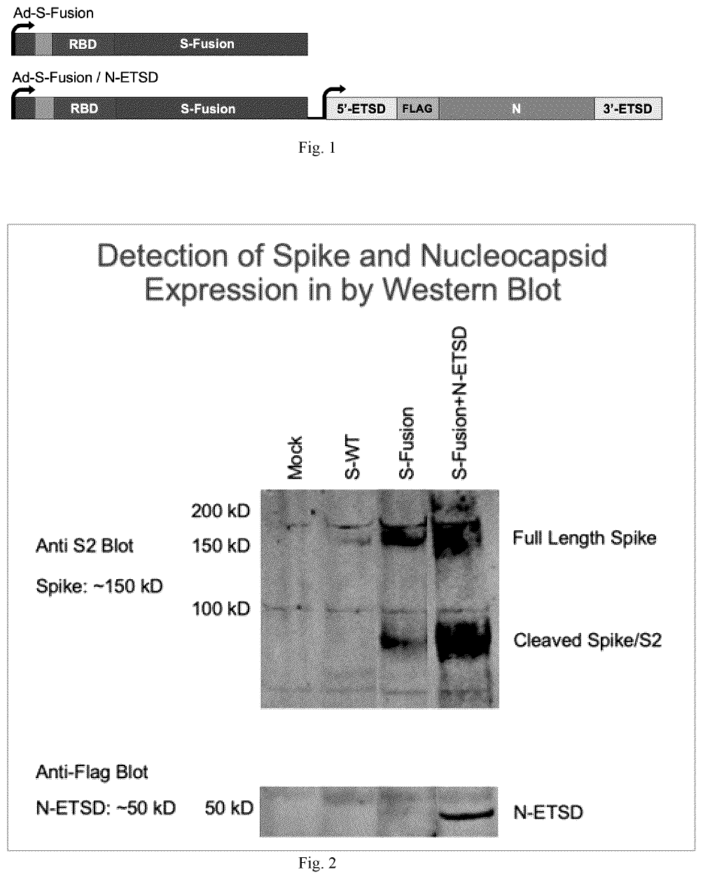

Disclosed herein are constructs that have been constructed and tested, a hAd5-COVID-19 vaccine construct E1-, E2b-, E3-hAd5 vector with SARS-CoV-2 (S/N) protein insert (FIG. 1). This construct has been tested in preclinical experiments, including in vitro expression (FIG. 2) and small animal immunogenicity.

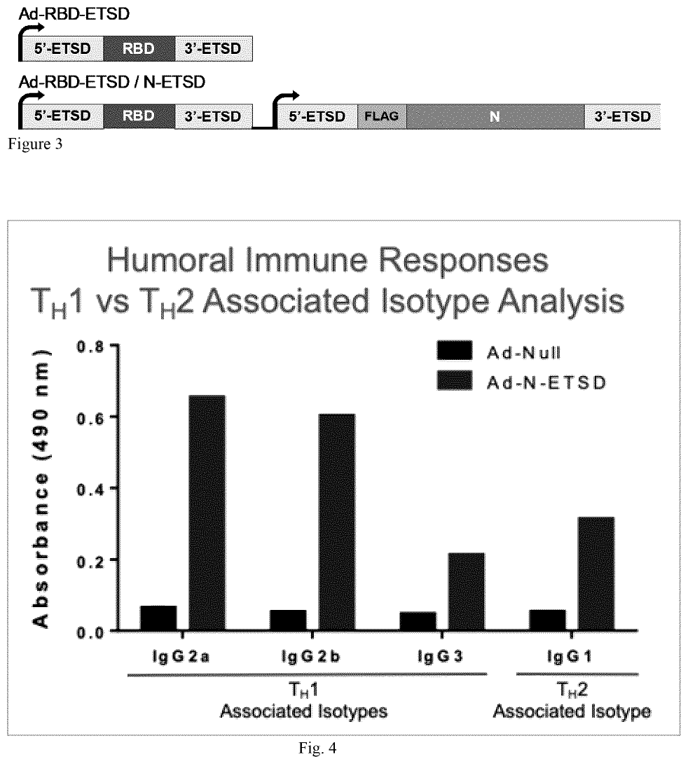

In addition, ImmunityBio has developed multiple COVID-19 constructs including RBD-alone, S1-alone, S1-fusion proteins, and combinations of RBD, S1 and S1 fusions with N. Preliminary in-vitro studies demonstrate that these constructs (FIG. 3) recognize convalescent serum antibodies and could serve as alternative vaccines following analysis of the two (2) constructs above (FIG. 1) which is intended to initiate in our first in human Phase 1b study.

Rationale for Inclusion of Nucleocapsid (N) in hAd5 Constucts for COVID-19

The nucleocapsid (N) protein of SARS-CoV-2 is highly conserved and highly expressed. Previous research with the related coronavirus that causes SARS demonstrated that N protein is immunogenic (Gupta, 2006), when integrated with intracellular trafficking constructs. To date, vaccine strategies in development all involve developing immunogenicity against spike (S) protein. However, very recent evidence in patients who recovered from COVID-19 demonstrates Th1 immunity generated against the nucleocapsid (N) (Grifoni, 2020). A second report by Grifoni et al. further confirmed that in the predictive bioinformatics model, T and B cell epitopes were highest for both spike glycoprotein and nucleoprotein (Grifoni, 2020). The present disclosure confirms the potential that combining S with N, that long-term cell-mediated immunity with a Th1 phenotype can be induced. The potential exists for this combination vaccine to serve as a long-term “universal” COVID-19 vaccine in light of mutations undergoing in S and the finding that the structural N protein is highly conserved in the coronavirus family. The clinical trial is designed to compare S alone versus S+N, to demonstrate safety and to better inform the immunogenicity of S and S+N. A single construct having S & N would be selected to induce potent humoral and cell mediated immunity.

### Immunogenicity Studies (Small Animal Model):

Homologous prime-boost immunogenicity in BALB-c mice. Mice have been treated with 1, 2 or 3 doses of the hAd5 COVID-19 vaccine and serum and splenocyte samples are being tested for SARS-CoV-2 antigen-specific immune responses. Serum is tested for anti-spike and anti-nucleocapsid antibody responses by ELISA. Splenocytes is tested for spike- and nucleocapsid-specific cell mediated immune responses by ELISPOT and intracellular cytokine simulation assays.

The results show promising immunogenic activity. In one embodiment, hAd5 [E1-,E2b-, E3-]N-ETSD, a vaccine containing SARS-CoV-2 nucleocapsid plus an enhanced T cell stimulation domain (ETSD), alters T cell responses to nucleocapsid. Mice were immunized subcutaneously (SC) with a dose of 1010 VP twice at 7-day intervals. Blood was collected at several time points and spleen was collected upon sacrifice in order to perform immunogenicity experiments. Splenocytes were isolated and tested for cell mediated immune (CMI) responses. The results showed that SARS-CoV-2 nucleocapsid antigen specific CMI responses were detected by ELISpot and flow cytometry analyses in the spleens of all the mice immunized with hAd5 [E1-, E2b-, E3-]N-ETSD vaccine but not vector control (hAd5 [E1-, E2b-, E3-] null) immunized mice.

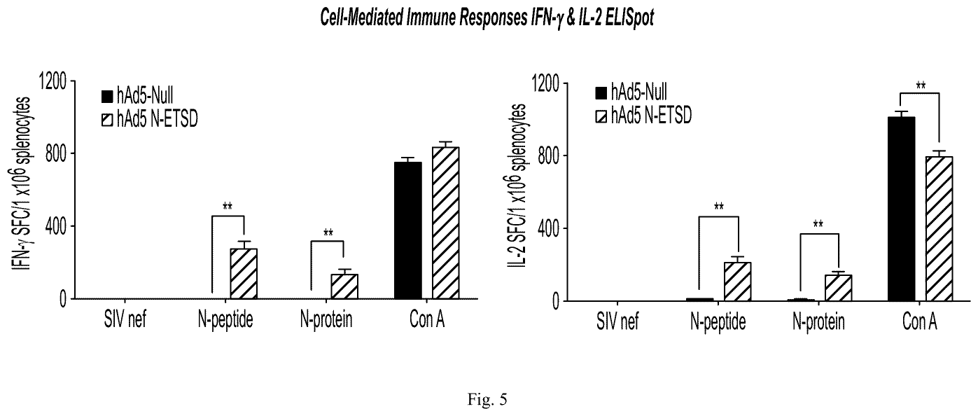

In addition, antibody responses were detected in all the mice immunized with hAd5 [E1-, E2b-, E3-]-N-ETSD vaccine but not vector control (Ad5 [E1-, E2b-, E3-]-null) immunized mice (FIG. 4 & FIG. 5). Additional studies to confirm and extend these results are ongoing.

### Enhanced RBD Cell Surface Expression:

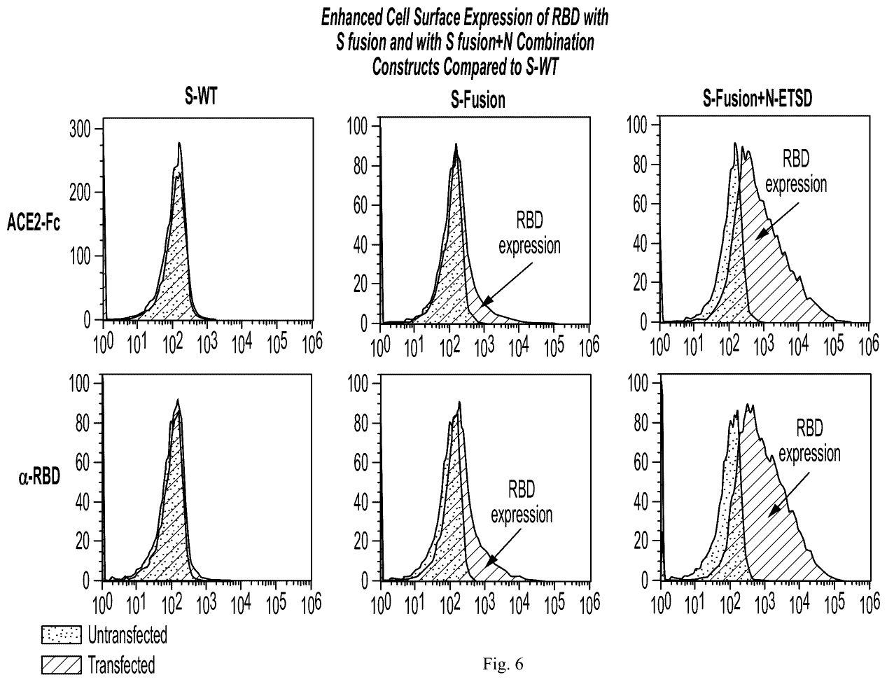

Further evidence of the potential enhancing immunogenicity value of N when combined with S was the surprising finding of enhanced surface expression of the RBD protein in 293 cells transfected with the N-ETSD+S construct as seen in FIG. 6. Expression and presentation of RBD appears to be highly important as evidenced by the recent report by Robbiani et al who showed that rare but recurring RBD-specific antibodies with potent antiviral activity were found in all individuals tested who had recovered from COVID-19 infections (Robbiani 2020).

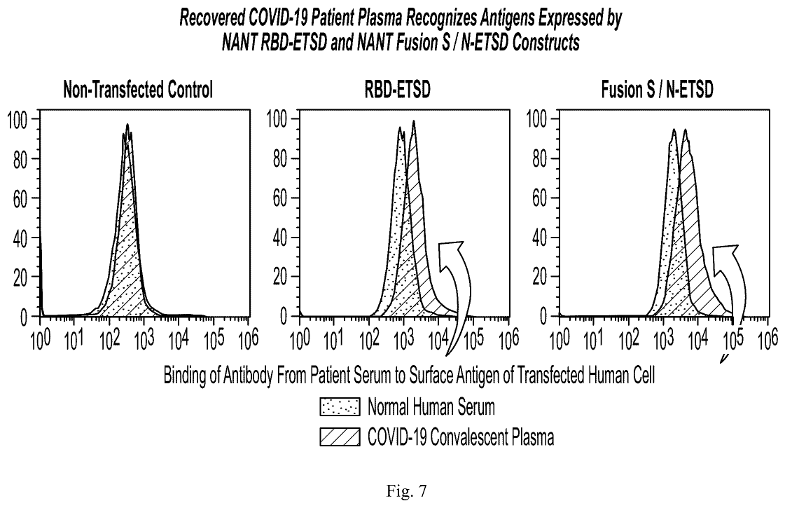

This finding of enhanced expression of RBD when N is combined with S-Fusion was coraborated in studies using plasma from a patient recovered from COVID-19 infection (FIG. 7). The alternative construct of RBD-ETSD could serve as alternative vaccines following analysis of the two (2) constructs above (FIG. 1) which is intended to initiate in human Phase 1b studies.

In summary, on the basis of enhanced expression and exposure of the RBD protein with S Fusion and S Fusion+N construct, both were tested in the hAd5 vector. Furthermore, on the basis of recent clinical data from patients recovered from COVID-19, as well as the corroborating preclinical data that the N construct induces long lasting CD4+ and Th1 cell-mediated immunity, this combination of S Fusion+N construct could provide long-lasting immunity beyond short term neutralizing antibodies.

### Immunogenicity Testing of Candidate COVID-19 Vaccine Constructs

Two (2) Adenovirus-based COVID-19 vaccine constructs will be tested in preclinical experiments, including in vitro expression; small animal immunogenicity, and non-human primate immunogenicity and efficacy.

Constructs description: ImmunityBio has generated two (2) second generation hAd5-based COVID-19 vaccine constructs for preclinical testing and clinical evaluation. First is a hAd5 vector with SARS-CoV-2 with spike protein insert (see FIG. 1). Second is E1-, E2b-, E3-hAd5 vector with SARS-CoV-2 wild type spike protein (S) insert and Nucleocapsid protein (N) insert containing an Endosomal-targeting domain sequence (ETSD) in the same vector backbone.

Immunogenicity Studies: Homologous prime-boost immunogenicity in mice was examined by treating Mice with 1, 2 or 3 doses of the ImmunityBio adenovirus vaccine candidates listed in FIG. 1 and serum and splenocyte samples will be tested for SARS-CoV-2 antigen-specific immune responses. Serum is being tested for anti-spike and anti-nucleocapsid antibody responses by ELISA. Splenocytes will be tested for spike- and nucleocapsid-specific cell mediated immune responses by ELISPOT and intracellular cytokine simulation assays. Data from these studies are disclosed throughout this disclosure.

SARS-CoV-2 Virus Neutralization Studies: Serum from the mice immunized during the course of the immunogenicity studies described above is used will be sent to a third-party subcontractor for SARS-CoV-2 neutralization studies to be performed in their ABSL-3 facility. Serum will be tested for COVID 19 virus neutralizing activity by mixing various dilutions of serum with COVID 19 virus, incubating the mixture, and then exposing the mixture to Vero cells to detect cytopathic effect (CPE). The last dilution that prevents CPE will be considered the endpoint neutralizing titer.

Immunogenicity and Efficacy Evaluation in Non-Human Primates (third-party subcontractor): Rhesus macaques will be treated with three doses of the ImmunityBio adenovirus vaccine candidates listed in FIG. 1. SARS-CoV-2 antigen-specific immune responses will be monitored in serum and PBMCs by ELISA, ELISPOT and ICS throughout the course of the therapy. Four weeks after the final vaccination, animals will be challenged with SARS-CoV-2 and monitored for disease hallmarks and virus shedding.

Phase Ib Clinical trial: ImmunityBio has submitted an IND for Phase Ib clinical trial testing of hAd5 [E1-, E 2b-, E3-] CoV-2 vaccine.

Study Design: This is a Phase 1b open-label study in adult healthy subjects. This clinical trial is designed to assess the safety, reactogenicity, and immunogenicity of the hAd5-COVID-19-S and hAd5-COVID-19-S/N vaccines. The hAd5-COVID-19-S and hAd5-COVID-19-S/N vaccines are hAd5 [E1-, E2b-, E3-] vector-based targeting vaccines encoding the SARS-CoV-2 Spike (S) protein alone or together with the SARS-CoV-2 nucleocapsid (N) protein. The hAd5 [E1-, E2b-, E3-] vector is the platform technology for targeted vaccines that has demonstrated safety in over 125 patients with cancer to date at doses as high as 5×1011 virus particles per dose. Co-administration of three different hAd5 [E1-, E2b-, E3-] vector-based vaccines on the same day at 5×1011 virus particles per dose each (1.5×1012 total virus particles) has also been demonstrated to be safe.

COVID-19 infection causes significant morbidity and mortality in a worldwide population. The hAd5-COVID-19-S and hAd5-COVID-19-S/N vaccines are designed to induce both a humoral and cellular response even in individuals with pre-existing adenoviral immunity. Thus, the potential exists for the hAd5-COVID-19-S and hAd5-COVID-19-S/N to induce anti-COVID-19 immunity and prevent or lessen the health impact of COVID-19 infection in healthy subjects.

Phase 1b Safety Analysis: In the initial safety analysis of phase 1b, a total of 40 healthy subjects will be divided into 4 dosing cohorts (cohorts 1A, 1B, 2A, 2B; n=10 for each cohort):
Cohort 1A-hAd5-COVID-19-S at 5×1010 viral particles (VP) per dose (n=10),
Cohort 1B-hAd5-COVID-19-S at 1×1011 VP per dose (n=10),
Cohort 2A-hAd5-COVID-19-S/N at 5×1010 VP per dose (n=10),
Cohort 2B-hAd5-COVID-19-S/N at 1×1011 VP per dose (n=10).

Each subject will receive a subcutaneous (SC) injection of hAd5-COVID-19-S or hAd5-COVID-19-S/N on Day 1 and Day 22 (ie, 2 doses). This dosing schedule is consistent with hAd5 [E1-, E2b-, E3-] vector-based vaccines currently in clinical trials. Cohorts 1-2 will enroll in parallel and may be opened at the same time or in a staggered manner depending upon investigational product supply. Subjects in cohorts 1A and 2A will complete the low-dose vaccination regimen first. After all subjects in cohorts 1A and 2A have completed at least a single dose and follow-up assessments during the toxicity assessment period through study day 8, enrollment will proceed if ImmunityBio Safety Review Committee (SRC) and at least one qualified infectious disease physician, independent of the Sponsor and trial, confirms absence of safety concerns. Subjects will then be enrolled in higher-dose cohorts 1B and 2B, and vaccinated. For all subjects, follow-up study visits will occur at days 8, 22, 29, 52, and at months 3, 6, and 12 following the final vaccination. Additional follow up for safety information will occur via telephone contact as noted in the Schedule of Events. The primary objectives of the initial safety phase 1b are to evaluate preliminary safety and reactogenicity of the hAd5-COVID-19-S and hAd5-COVID-19-S/N vaccines. The secondary objectives are to evaluate the extended safety and immunogenicity of the hAd5-COVID-19-S and hAd5-COVID-19-S/N vaccines.

### Expanded Phase 1b: Safety and Immunogenicity for Construct Selection

Phase 1b expansion will proceed if the SRC determines it is safe to do so based on a review of safety data from the phase 1b safety assessment. In phase 1b expansion, a total of 60 healthy subjects will be divided into 4 dosing cohorts (cohorts 1A, 1B, 2A, 2B; n=15 for each cohort):
Cohort 1A-hAd5-COVID-19-S at 5×1010 VP per dose (n=15)
Cohort 1B-hAd5-COVID-19-S at 1×1011 VP per dose (n=15)
Cohort 2A-hAd5-COVID-19-S/N at 5×1010 VP per dose (n=15)
Cohort 2B-hAd5-COVID-19-S/N at 1×1011 VP per dose (n=15)

Each subject will receive a SC injection of hAd5-COVID-19-S or hAd5-COVID-19-S/N on Day 1 and Day 22 (ie, 2 doses). For all subjects, follow-up study visits will occur at days 8, 22, 29, 52, and at months 3, 6, and 12 following the final vaccination. Additional follow up for safety information will occur via telephone contact as noted in the Schedule of Events. The primary objective of the expanded phase 1b is to select the most immunogenic construct between hAd5-COVID-19-S and hAd5-COVID-19-S/N and dose level as determined by changes in humoral and cellular immunogenicity indexes. The secondary objectives are to assess safety and reactogenicity of hAd5-COVID-19-S and hAd5-COVID-19-S/N.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). Most preferably, the recombinant virus is administered via subcutaneous or subdermal injection. However, in other contemplated aspects, administration may also be intravenous injection. Alternatively, or additionally, antigen presenting cells may be isolated or grown from cells of the patient, infected in vitro, and then transfused to the patient.

In one aspect of any of the embodiments described above or elsewhere herein, the composition is formulated in a pharmaceutically acceptable excipient suitable for administration to a subject.

It is still further contemplated that the recombinant viruses and yeasts contemplated herein may further comprises a sequence that encodes at least one of a co-stimulatory molecule, an immune stimulatory cytokine, and a protein that interferes with or down-regulates checkpoint inhibition. For example, suitable co-stimulatory molecules include CD80, CD86, CD30, CD40, CD30L, CD40L, ICOS-L, B7-H3, B7-H4, CD70, OX40L, 4-1BBL, GITR-L, TIM-3, TIM-4, CD48, CD58, TL1A, ICAM-1, and/or LFA3, while suitable immune stimulatory cytokine include IL-2, IL-12, IL-15, IL-15 super agonist (N803), IL-21, IPS1, and/or LMP1, and/or suitable proteins that interfere include antibodies against or antagonists of CTLA-4, PD-1, TIM1 receptor, 2B4, and/or CD160.

It should be appreciated that all of the above noted co-stimulatory genes are well known in the art, and sequence information of these genes, isoforms, and variants can be retrieved from various public resources, including sequence data bases accessible at the NCBI, EMBL, GenBank, RefSeq, etc. Moreover, while the above exemplary stimulating molecules are preferably expressed in full length form as expressed in human, modified and non-human forms are also deemed suitable so long as such forms assist in stimulating or activating T-cells. Therefore, muteins, truncated forms and chimeric forms are expressly contemplated herein.

The immunotherapeutic compositions disclosed herein may be either “prophylactic” or “therapeutic”. When provided prophylactically, the compositions of the present disclosure are provided in advance of the development of, or the detection of the development of, a coronavirus disease, with the goal of preventing, inhibiting or delaying the development of the coronavirus disease; and/or generally preventing or inhibiting progression of the coronavirus disease in an individual. Therefore, prophylactic compositions can be administered to individuals that appear to be coronavirus disease free (healthy, or normal, individuals), or to individuals who has not yet been detected of coronavirus. Individuals who are at high risk for developing a coronavirus disease, may be treated prophylactically with a composition of the instant disclosure.

When provided therapeutically, the immunotherapy compositions are provided to an individual who is diagnosed with a coronavirus disease, with the goal of ameliorating or curing the coronavirus disease; increasing survival of the individual; preventing, inhibiting, reversing or delaying development of coronavirus disease in the individual.

The contemplated subject matter further includes methods for administering a vaccine to a patient by more than one route of administration to induce both local and systemic immune responses to the vaccine. The contemplated subject matter also includes compositions and methods for assaying the presence or absence of the relevant antibodies (e.g., anti-SARS-CoV2 antibodies) in a patient sample (e.g., saliva, nasal mucosa, alimentary mucosa, or serum). The antibody status in the patient's sample may be used to assess the need for an additional vaccine dose (e.g., a booster dose/shot).

In addition to the coveted molecular epitopes presented in a vaccine, the route of administration of the vaccine as well as the regimen for administering additional (i.e., booster) doses of the vaccine, can also affect whether or not the patient's immune response is robust enough to establish protection.

For an emerging virus such as the severe acute respiratory syndrome (SARS)-like coronavirus (SARS-CoV2), the duration of immunity (both humoral and cell-mediated) in a patient recovered from a SARS-CoV2 infection is not yet completely known, and furthermore, a vaccine protocol has not yet been tested across a varied population. Considering the current SARS-CoV2 pandemic and the high rate of transmission for the SARS-CoV2 virus, there is a need for a robust vaccination protocol and effective testing for the virus or immunity to the virus (e.g., presence of anti-SARS-CoV2 antibodies).

Vaccine Administration. The presently disclosed contemplated methods for inducing immunity in a patient include administering a vaccine by at least oral administration, and preferably by oral administration and by injection to the blood supply. Many vaccines are given via the intramuscular (IM) route to optimize immunogenicity with the direct delivery of the vaccine to the blood supply in the muscle to induce systemic immunity. The IM administration is typically preferred over subcutaneous (SC) injection which is more likely to have adverse reactions at the injection site than IM injections.

In addition to IM injection, induction of mucosal immunity has been reported to be essential to stop person-to-person transmission of pathogenic microorganisms and to limit their multiplication within the mucosal tissue. Furthermore, for protective immunity against mucosal pathogens, (e.g., SARS coronaviruses) immune activation in mucosal tissues instead of the more common approach of tolerance to maintain mucosal homeostasis allows for enhanced mucosal immune responses and better local protection. For example, nasal vaccination (delivery of a vaccine by nasal administration) induces both mucosal immunity as well as systemic immunity. See, e.g., Fujkuyama et al., 2012, Expert Rev Vaccines, 11:367-379 and Birkhoff et al., 2009, Indian J. Pharm. Sci., 71:729-731.

In order to induce both mucosal and systemic immunity in a patient, embodiments of the present disclosure include providing a vaccine to the patient by at least administration to the nasal mucosa, oral mucosa, and/or alimentary mucosa of the patient. In some embodiments, the routes of administration include administering the vaccine to the nasal mucosa, oral mucosa, and/or alimentary mucosa of the patient together with injection into the blood supply (e.g., intramuscular (IM), intravenous (IV), or subcutaneous (SC)). As used herein, oral administration of a vaccine composition includes nasal injection, nasal inhalation, ingestion by mouth, and administration (e.g., inhalation, ingestion, injection) to the alimentary mucosa. Preferably, the routes of administering the vaccine include oral administration selected from delivery to the alimentary mucosa, nasal injection, nasal inhalation, ingestion by mouth, or inhalation by mouth together with administration by intramuscular (IM) injection.

Notably, the vaccine administered for inducing immunity in the mucosal tissue of a patient is a vaccine against SARS-CoV2. In exemplary embodiments, the vaccine a replication defective adenovirus construct, comprising an E1 gene region deletion and an E2b gene region deletion. In certain embodiments the adenovirus comprises a sequence (e.g. SEQ ID NO:11) encoding a SARS-CoV2 spike protein antigen with at least 80% (e.g., at least 85%, at least 90%, at least 95%, at least 96%, at least 97%, at least 98%, at least 99%) primary sequence identity to SEQ ID NO:10. In certain embodiments the adenovirus comprises a sequence (e.g. SEQ ID NO:13) encoding a SARS-CoV2 spike protein antigen with at least 80% (e.g., at least 85%, at least 90%, at least 95%, at least 96%, at least 97%, at least 98%, at least 99%) primary sequence identity to SEQ ID NO:12. In certain embodiments, the adenovirus includes a sequence encoding a soluble ACE2 protein coupled to an immunoglobulin Fc portion, forming an ACE2-Fc hybrid construct that may also include a J-chain portion, as disclosed in U.S. Ser. No. 16/880,804 and U.S. 63/016,048, the entire contents of both of which are herein incorporated by reference. In other exemplary embodiments, the SARS-CoV2 vaccine (e.g., an adenovirus construct) includes a mutant variant of a recombinant soluble ACE2 protein (e.g., SEQ ID NO: 9), wherein the mutant variant has at least one mutated amino acid residue (e.g., by substitution) that imparts an increased binding affinity of the ACE2 protein for the RBD protein domain of the SARS-CoV2 spike protein as disclosed in U.S. 63/022,146, the entire content of which is herein incorporated by reference. In another exemplary embodiment, the SARS-CoV2 vaccine (e.g., an adenovirus construct) includes a CoV2 nucleocapsid protein or a CoV2 spike protein fused to an endosomal targeting sequence (N-ETSD), as disclosed in U.S. Ser. No. 16/883,263 and U.S. 63/009,960, the entire contents of both of which are herein incorporated by reference. Additionally or alternatively, the SARS-CoV2 vaccine includes modified yeast cells (e.g., Saccharomyces cerevisiae) genetically engineered to express coronaviral spike proteins on the yeast cell surface thereby creating yeast presenting cells to stimulate B cells (e.g., humoral immunity) as disclosed in U.S. 63/010,010.

In some embodiments, more than one vaccine composition as disclosed herein may be administered to a patient to induce immunity to SARS-CoV2. For example, a patient may be administered genetically modified yeast cells expressing corona viral spike proteins as a single type of vaccine, or the genetically modified yeast cells may be administered together or concurrently with one or more SARS-CoV2 adenovirus constructs as disclosed herein.

Monitoring presence of antibodies. The contemplated subject matter also includes monitoring or assessing a patient's immune response either to a vaccine administered as disclosed herein (e.g., by oral administration and injection into the blood supply), or to infection by the virus. In particular, disclosed herein are compositions and methods for assessing the continued presence of antibodies in a patient's respiratory and digestive mucosa following infection with SARS-CoV2 or following inoculation against SARS-CoV2 with administration of a SAR coronavirus vaccine.

For assaying a sample from a patient having received a vaccine against a pathogenic infection (e.g., targeting SARS-CoV2) and/or having been infected with a virus (e.g., SARS-CoV2), the presence of antibodies against the pathogen may be carried out using any one of many diagnostic tests. In some embodiments, the diagnostic test is a cell viability assay that allows for the detection of antibodies in the presence of antigen. Diagnostic tests using a cell viability assay for anti-SARS-CoV2 antibody detection are disclosed in U.S. 62/053,691, the entire contents of which are herein incorporated by reference. The cellular diagnostic assay relies on the expression of the target receptor for a given pathogen (e.g., ACE2 for SARS-CoV2 infection) on the surface of an immune effector cell line (e.g., killer T cells, natural killer cells, NK-92® cells and derivatives thereof, etc.) and the expression of the pathogen ligand (e.g., Spike proteins for SARS-CoV2 infection) on the surface of a surrogate cell line (e.g., HEK293 cells or SUP-B15 cells).

Additional diagnostic tests using recombinant protein variants of the ACE2 protein (the human receptor targeted by SARS-CoV2 spike protein) are disclosed in U.S. Ser. No. 16/880,804, the entire contents of which are herein incorporated by reference.

Antibody testing in saliva samples. In order to more easily monitor a patient for the presence of anti-pathogen antibodies, assaying a saliva sample from the patient allows for expedited sample collection, increased patient participation, and may allow for the patient to obtain the sample themselves and either mail or transport the sample to the lab for testing. However, in order to assay saliva for the presence of neutralizing antibodies against SARS-CoV2, it may be necessary to stabilize proteins in the saliva against degradation during transport and storage after sample collection prior to testing.

Upon collection of the saliva sample, the saliva is placed into a preservative solution to stabilize the components (e.g., anti-SARS CoV2 antibody or viral spike protein) therein. Preservatives for biological samples are disclosed, for example, in Cunningham & al. (2018) report (“Effective Long-term Preservation of Biological Evidence,” U.S. Department of Justice grant #2010-DN-BX-K193) and U.S. Pat. No. 6,133,036 to Putcha et al. For example, a stabilizing preservative solution for a patient's saliva sample may include any one of glutaraldehyde, sodium benzoate, citric acid, propyl gallate, EDTA, zinc, actin, chitosan, parabens, sodium azide, and any combination thereof.

In specific embodiments, saliva samples may be mixed with stabilizing preservative solutions of glutaraldehyde to achieve a final glutaraldehyde concentration between 0.1% (w/v) and 2.0% (w/v), for example about 0.2% (w/v), about 0.3% (w/v), about 0.4% (w/v), about 0.5% (w/v), about 0.6% (w/v), about 0.7% (w/v), about 0.8% (w/v), about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), or about 1.9% (w/v).

In additionally or alternatively embodiments, saliva samples may be mixed with a stabilizing preservative solution of about 0.10% to about 1.00% sodium benzoate (weight/volume of sample) and/or about 0.025% to about 0.20% citric acid (weight/volume of sample). For example, the saliva sample may be mixed with 0.10%, 0.20%, 0.30%, 0.40%, 0.50%, 0.60%, 0.70%, 0.80%, 0.90%, or 1.00% w/v sodium benzoate. In additional embodiments, the saliva sample is mixed a stabilizing preservative solution of at least 0.5 mg/mL (for example, at least 0.6 mg/mL, at least 0.7 mg/mL, at least 0.8 mg/mL, at least 0.9 mg/mL, at least 1 mg/mL, at least 1.5 mg/mL, at least 2 mg/mL, at least 2.5 mg/mL, at least 3 mg/mL, at least 3.5 mg/mL, at least 4 mg/mL, at least 4.5 mg/mL, or even 5 mg/mL) of benzoic acid and/or at least 0.2 mg/mL (for example, at least 0.2 mg/mL, at least 0.25 mg/mL, at least 0.3 mg/mL, at least 0.35 mg/mL, at least 0.40 mg/mL, at least 0.50 mg/mL, at least 0.75 mg/mL, at least 1.0 mg/mL, at least 1.25 mg/mL, at least 1.5 mg/mL, at least 1.75 mg/mL, or even 2.0 mg/mL) of citric acid. As used herein, “benzoic acid” is interchangeable with benzoate salt (e.g., sodium benzoate) and “citric acid” is interchangeable with citrate salt (e.g., sodium citrate).

The saliva samples with preservatives as described above are stable for storage at temperatures between 15° C. and 40° C. for at least one hour (e.g., at least 5 hours, at least 10 hours, at least 12 hours, at least 24 hours, at least 48 hours, or even 36 hours). Therefore, disclosed herein is a method of preserving a saliva sample for neutralizing antibody testing, the method including mixing the saliva sample with the stabilizing solution made of one or more of glutaraldehyde, sodium benzoate, citric acid, propyl gallate, EDTA, zinc, actin, chitosan, parabens, and/or sodium azide and storing between 15° C. and 25° C. for at least one hour, and up to 2, 3, 4, 5, 6, 7, 8, 9, 10, 11, 12, 13, 14, 15, 16, 17, 18, 19, 20, 21, 22, 23, 24, 36, or 48 hours. In some embodiments, the saliva sample is mixed with a glutaraldehyde concentration between 0.1% (w/v) and 2.0% (w/v), and the glutaraldehyde-saliva is stored between 15° C. and 25° C. In certain embodiments, the glutaraldehyde-saliva may further comprise citric acid and/or benzoic acid at a concentration of as disclosed herein.

Aragonite. In some embodiments, any antibody proteins or any specific antibody protein may be captured from the saliva sample with oolitic aragonite particles. For example, the saliva preserving solution of glutaraldehyde, sodium benzoate and citric acid, propyl gallate, EDTA, zinc, actin, chitosan, parabens, sodium azide, and any combination thereof as disclosed herein, may also include oolitic aragonite (calcium carbonate, CaCO₃) particles. Use of aragonite particles for binding to proteins is disclosed, for example, in U.S. Ser. No. 16/858,548 and PCT/US20/29949, the entire contents of both of which are herein incorporated by reference. Accordingly, aragonite particles may be added to that have been modified to capture (e.g., bind to) any antibodies present in the saliva sample or specifically capture an antibody against a specific antigen. For example, aragonite may be functionalized with moieties capable of binding to an immunoglobulin (Ig) protein. Preferably, the Ig protein is an immunoglobulin A (IgA), immunoglobulin G (IgG), or immunoglobulin E (IgE) protein. More preferably, the aragonite is functionalized to bind to an IgA protein. Most preferably, the aragonite particles are functionalized with moieties capable of binding to specific antibodies. For example, the aragonite particles may be coupled with a moiety specific to anti-SARS-CoV2 antibodies. Preferably, the aragonite particle is coupled with a recombinant ACE2 protein as disclosed, for example, in U.S. Ser. No. 16/880,804, supra. In typical embodiments, the aragonite particle is coupled with a recombinant human ACE2 protein having at least 85%, at least 90%, or at least 95% sequence identity to SEQ ID NO: 8.

In additional or alternative embodiments, the aragonite particle is functionalized (e.g., coupled to) a recombinant soluble ACE2 protein (e.g., SEQ ID NO: 9). For more efficient capture or binding of an anti-SARS-CoV2 antibody or the spike protein of SARS CoV-2, the recombinant soluble ACE2 may be mutated to form ACE2 variants having higher binding affinities for SARS-CoV2 spike protein (e.g., the RBD domain of the spike protein). These ACE2 variant mutants of the recombinant soluble ACE2 protein include T27F, T27W, T27Y, D30E, H34E, H34F, H34K, H34M, H34W, H34Y, D38E, D38M, D38W, Q24L, D30L, H34A, and/or D355L.

As used herein, the term “functionalized” refers to coupling or binding of a moiety to the aragonite particle thereby imparting any function of the coupled moiety to the aragonite particle. For example, the aragonite particle may be functionalized with a protein moiety. Methods for preparing and using aragonite particle beads are disclosed in U.S. Ser. No. 16/858,548 and PCT/US20/29949. In some embodiments, the aragonite composition includes a plurality of aragonite particle beads. Preferably, the plurality of aragonite particle beads have an average particle size of between 100 nm to 1 mm,

In some embodiments a protein moiety is coupled directly to the natural, untreated surface of aragonite particles. Aragonite particles approximately 2-3% amino acid content including aspartic acid and glutamic acid rendering the aragonite surface hydrophilic. Accordingly, in some embodiments, protein moieties may be directly coupled to the surface of the aragonite particles.

In alternative embodiments, the aragonite particle surface may be treated to modify the binding surface. For example, treatment with stearic acid (i.e., octadecanoic acid) provides for a hydrophobic surface, as disclosed in U.S. Ser. No. 16/858,548 and PCT/US20/29949. For protein loading, treatment of the aragonite with phosphoric acid forms lamellar structures. Additional conjugation techniques for coupling reactive groups to the amino acid surface of aragonite are known in the art as disclosed, for example, in Bioconjugate Techniques, Third Edition, Greg T. Hermanson, Academic Press, 2013.

Monitoring of Vaccine Protocol. Patients who do not show sufficient titers of (e.g., presence of) neutralizing antibody in their saliva may be sent oral dosages of the respective vaccine (e.g., a SARS-CoV2 vaccine as disclosed herein). The patients inhale or ingest these vaccine dosages, and then two weeks later send another saliva sample-prepared and stored in the same manner as above—to the test facility to confirm that the oral vaccine dose has restored their anti-SARS-CoV2 antibody (e.g., IgA) titers.

Accordingly, in additional embodiments, a kit for collecting a saliva sample from a patient includes a collection container with the saliva preservative solution as disclosed herein. For example, the kit includes a collection container with a solution of any of one or combination of glutaraldehyde, sodium benzoate and/or citric acid, propyl gallate, EDTA, zinc, actin, chitosan, parabens, and sodium azide. The kit may also include adhesive packaging and/or mailing supplies in order to secure the collection container with the saliva sample for transport or mailing. In some embodiments, the kit may also include at least one dose of the vaccine for oral administration.

Recited ranges of values herein are merely intended as a shorthand referring individually to each separate value falling within the range. Unless otherwise indicated herein, each individual value is incorporated into the specification as if it were individually recited herein.

### EXAMPLES

The advantageous features of the compositions and methods described herein are further illustrated (but not limited) by the following examples.

### Example 1

Two groups of Rhesus macaques (5 per group) were immunized subcutaneously on day 0 with an adenoviral anti-SARS-CoV2 vaccine as described above. Blood was drawn from each macaque before immunization. On day 14, one group of macaques (Group 1) received another subcutaneous booster injection of the same vaccine, while another group (Group 2) received an oral vaccine as described herein (E1-/E2b-Ad5 with SEQ ID NO:11 or SEQ ID NO:13). On day 28, both groups received an oral vaccine booster dose. Two macaques (Control) were vaccinated at the indicated time points with shams. Blood was drawn on days 14, 21, 28, 35, & 42.

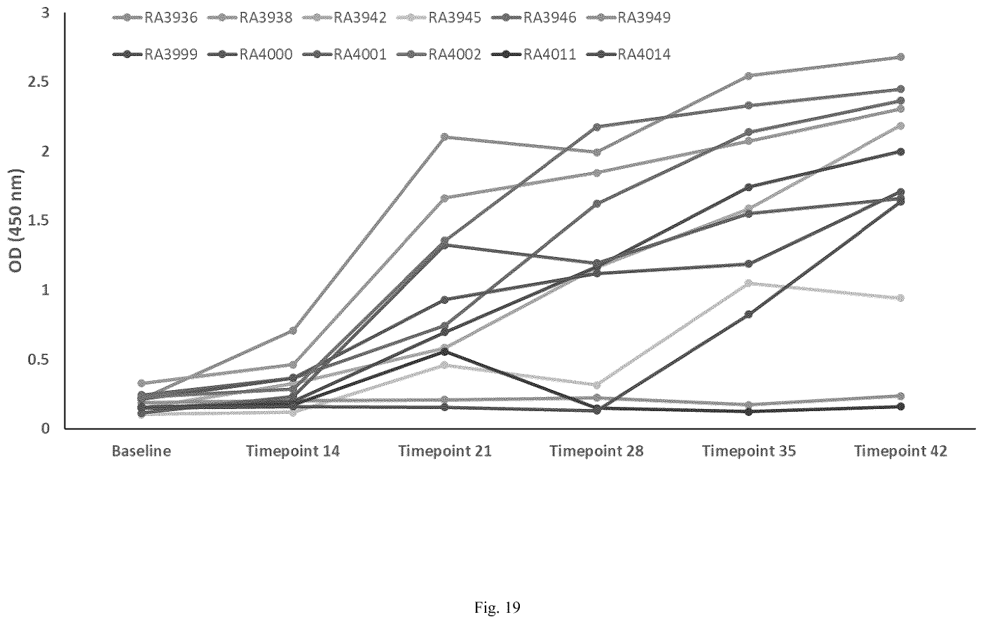

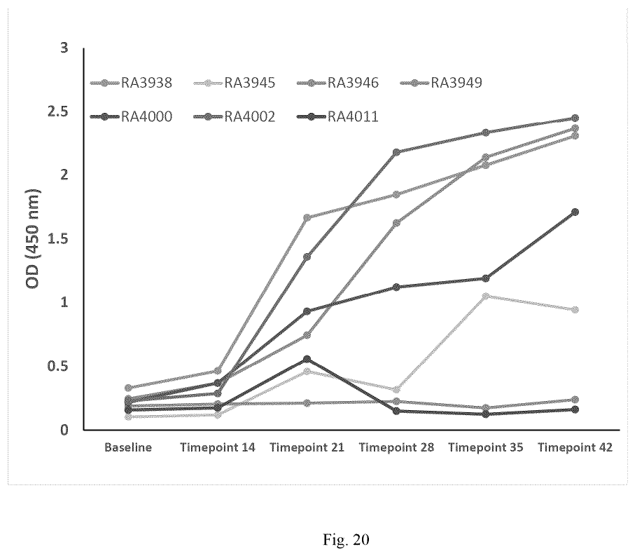

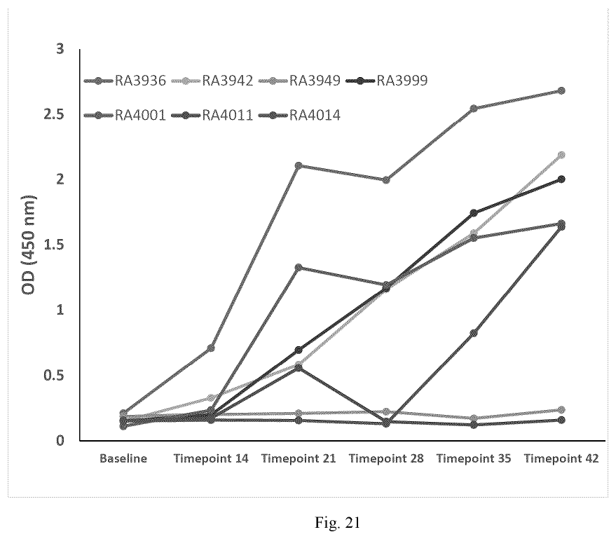

Serum samples drawn at the indicated time points from these macaques was then assessed by ELISA for anti-spike protein IgG and IgM seroreactivity. Briefly, 96 well EIA/RIA plates (ThermoFisher, Cat #07-200-642) were coated with 50 μL/well of 1 g/mL solution of purified recombinant SARS-CoV-2-derived Spike protein (S-Fusion. ImmunityBio, Inc.) suspended in coating buffer (0.05 M Carbonate-Bicarbonate, pH 9.6) and incubated overnight at 4° C. Individual 96 well plates were prepared for each immunoglobulins type (IgG or IgM) by washing three times each per well with 150 μL of TPBS solution (PBS+0.05% Tween 20). 100 μL/well of blocking solution (2% non-fat milk in TPBS) was then added and incubated for 1 hour at room temperature (RT). Plasma and serum samples were heat-inactivated at 56° C. for 1 hour before use. Serial dilutions of plasma, serum or antibody samples were prepared in 1% non-fat milk in TPBS. Plates were washed as described above and 50 μL/well of each serial dilution were added to the plate and incubated at RT for 1 hour. Plates were washed three times with 200 μL of TPBS. Dilutions (1:6000) of each goat anti-Human IgG (H+L) Cross-Adsorbed, HRP, Polyclonal; or Goat anti-Human IgM (Heavy chain) Cross-Adsorbed Secondary Antibody, HRP (ThermoFisher, Cat #62-842-0 or A18841 respectively) were 1 prepared in 1% non-fat milk/TPBS and 50 μL/well of these secondary antibodies were added in separate reactions/plates per immunoglobulin type (IgG or IgM) and incubated for 1 hour at RT. Plates were washed three times with 200 μL of TPBS. One component (3,3′,5,5′-tetramethylbenzidine (TMB) substrate, 50 μL/well, VWR, Cat #100359-156) was added to each well and incubated at RT for 10 minutes and then the reaction was stopped by addition of 50 μL/well of 1N Sulfuric acid (H₂SO₄). The optical density at 450 nm was measured with a Synergy 2 plate reader (BioTek Instruments, Inc). Data were analyzed using Prism 8 (GraphPad Software, LLC), and shown in FIG. 19.

### Example 2

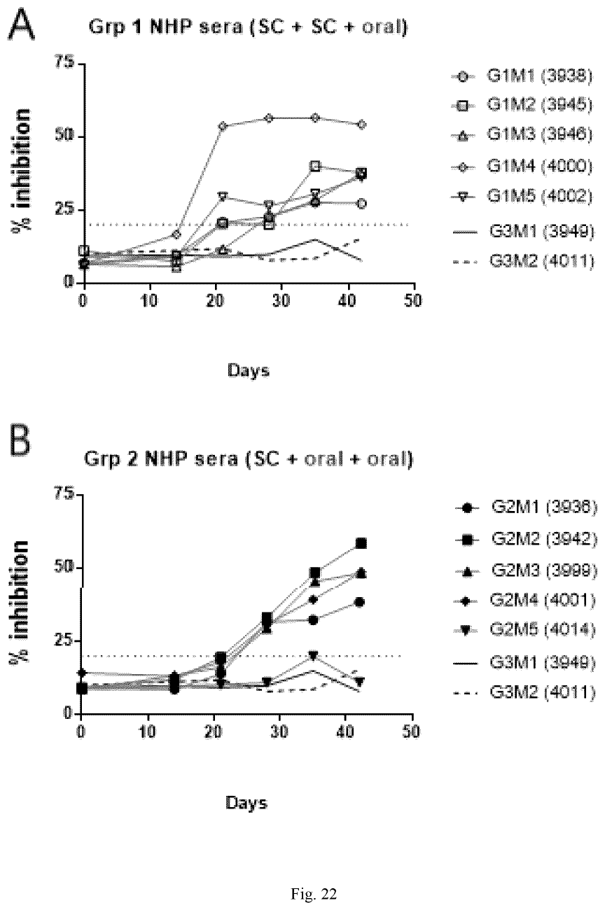

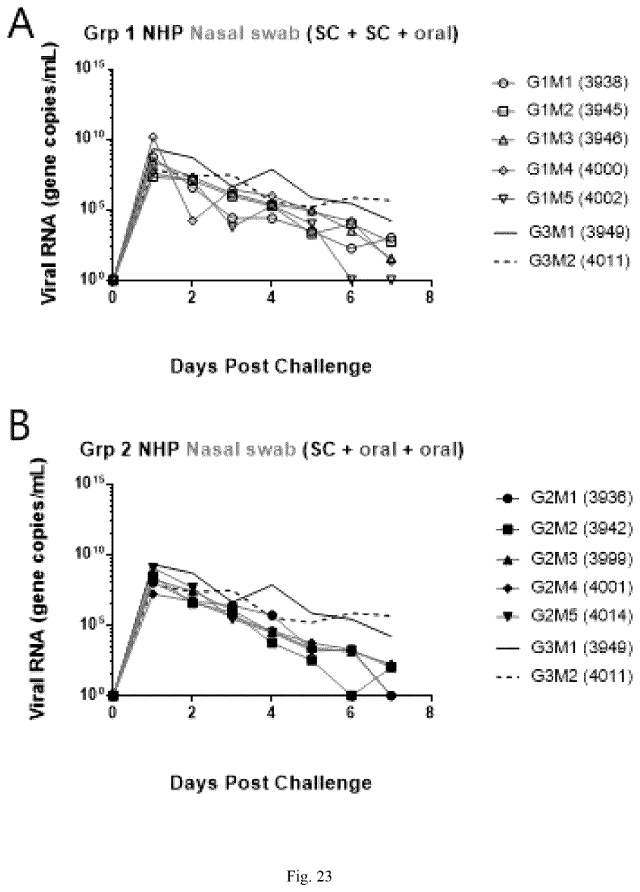

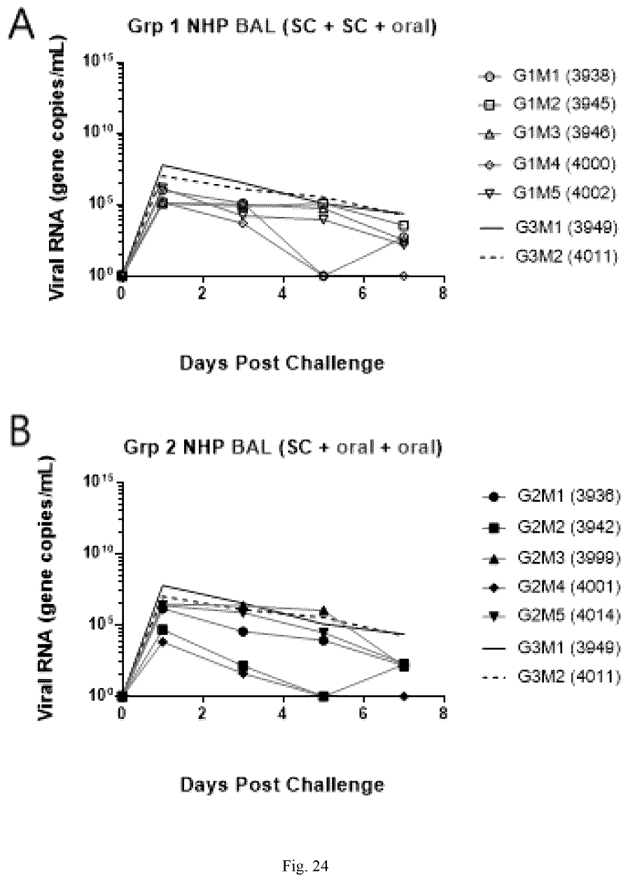

On day 56, the macaques were challenged with respiratory exposure to the SARS-CoV2 virus. Nasal swabs were collected daily from these macaques on days 56-63. Bronchoalveolar lavage (BAL) fluid was collected on days 57, 59, 61, & 63. The ability of serum to inhibit SARS-CoV2 infectivity from the samples collected is shown in FIG. 22. As can be seen, the sera from both the Group 1 and Group 2 macaques inhibited infectivity, with later collected sera inhibited more powerfully than early collected sera. Sera from control macaques had no inhibititory effect at any time point tested. Viral load over time in the nasopharynx is shown in FIG. 23. Viral load over time in the lungs is shown in FIG. 24.

### Example 3

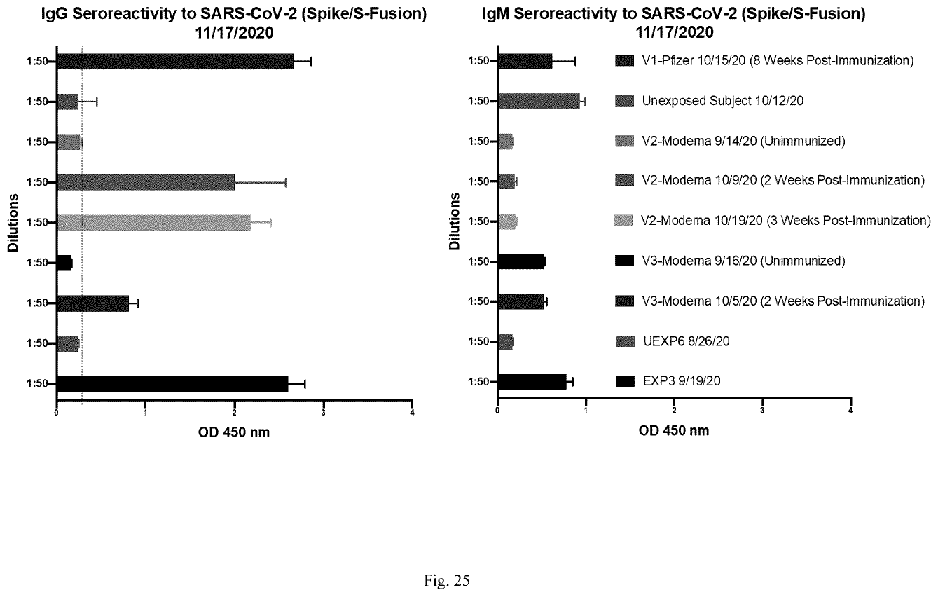

Serum samples from various human volunteers who have received various experimental anti-SARS-CoV2 vaccines were collected and assayed by ELISA as described above for IgG and IgM seroreactivity against SARS-CoV2 S protein. The results are shown in FIG. 25.

### Example 4

Human volunteers were divided into three cohorts. Cohort 1 (10 individuals) was immunized by subcutaneous injection with 5×10¹⁰ viral particles of a vaccine as described herein (E1-/E2b-Ad5 containing SEQ ID NO:11 or SEQ ID NO: 13). Cohort 2 (10 individuals) was immunized by subcutaneous injection with 10¹¹ viral particles of a vaccine as described herein. Cohort 3 (15 individuals) was immunized by subcutaneous injection with 10¹¹ viral particles of a vaccine as described herein (or 5×10¹⁰ viral particles if safety concerns indicated a lower dose). Blood was drawn from each volunteer on the same day as the initial prime vaccination was administered. Blood was drawn again on days 8, 15, & 22. A booster injection of the same vaccine was administered on day 22.

ELISpot tests were run on the blood collected on days 1 & 15 to assess cell-mediated immunity against SARS-CoV2. 400,000 viable PBMCs from each blood draw per well (Cellometer K2 w/AO/PI viability stain) were stimulated with empty medium, SARS-CoV2 S, SARS-CoV2 N, SARS-CoV2 M, CD3/CD28/CD2, and CEFT. After 48 hrs of stimulation, supernatants were frozen (−80° C.) for later testing.

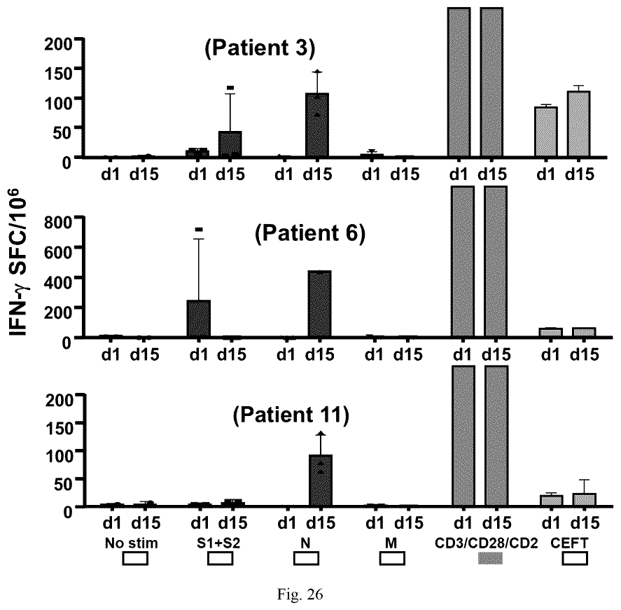

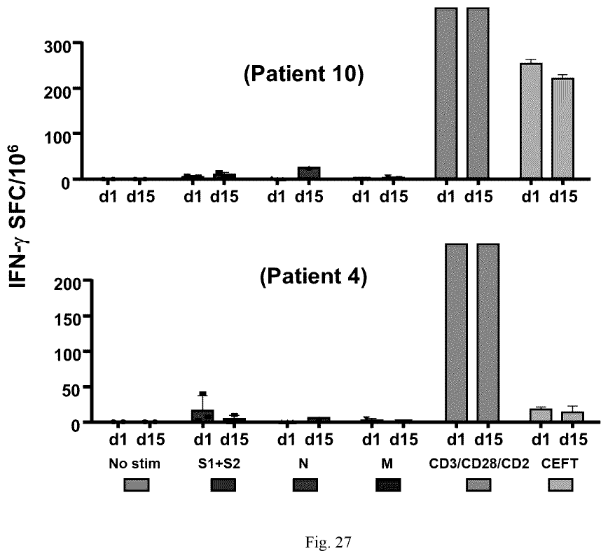

FIG. 26 shows the results of this test from Th1 N-responsive patients 3, 6, & 11. FIG. 27 shows results from patient 4 (N-unresponsive) and patient 10 (weakly Th1 N-responsive). None of these patients showed a Th2 response to N.

### Example 5

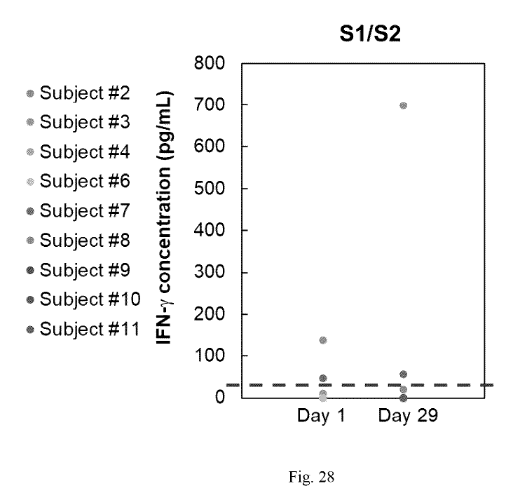

Human volunteers received 5×10¹⁰ viral particles of vaccine by subcutaneous injection on day 1 of the study, and again on day 22. Blood was drawn from each subject on days 1 and 29. These blood samples were assayed for immune reactivity to the SARS-CoV2 S protein by the methods described in co-pending U.S. 63/124,979 (filed 14 Dec. 2020). FIG. 28 shows the results of these assays. As can be seen, subject #8 shown a level of immune response to the S protein above the level of detection already on the first day of the experiment, indicating that this particular individual had already been previously infected with SARS-CoV2. The course of immunization produced a notable increase in immune response relative to baseline. This result constitutes in vivo evidence that the vaccines described herein can serve as vaccine boosts even to individuals whose immunity derives from some other source than prior immunization with the vaccines described herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be apparent to those skilled in the art that many more modifications besides those already described are possible without departing from the inventive concepts herein. The present disclosure, therefore, is not to be restricted except in the scope of the appended claims. Moreover, in interpreting both the specification and the claims, all terms should be interpreted in the broadest manner consistent with the context.

### Example 6: The hAd5 [E1-, E2b-, E3-] Platform and Constructs

For studies here, the next generation hAd5 [E1-, E2b-, E3-] vector was used (FIG. 1c) to create viral vaccine candidate constructs. As shown in FIG. 8d-h, a variety of constructs were created:

FIG. 8(d): S WT: S protein comprising 1273 amino acids and all S domains: extracellular (1-1213), transmembrane (1214-1234), and cytoplasmic (1235-1273) (Unitprot PODTC2);

FIG. 8(e): S RBD-ETSD: S Receptor Binding Domain with an Enhanced T-cell Stimulation Domain (ETSD);

FIG. 8(f): S Fusion: S optimized to enhance surface expression and display of RBD;

FIG. 8(g): N-ETSD: The nucleocapsid (N) sequence with the ETSD; and

FIG. 8(h): Bivalent S-Fusion+N-ETSD;

S-WT+N-ETSD and S RBD-ETSD+N-ETSD constructs were also produced, but are not shown.

### Example 7: Enhanced HEK 293T Cell-Surface Expression of RBD Following Transfection with Ad5 S-Fusion+N-ETSD

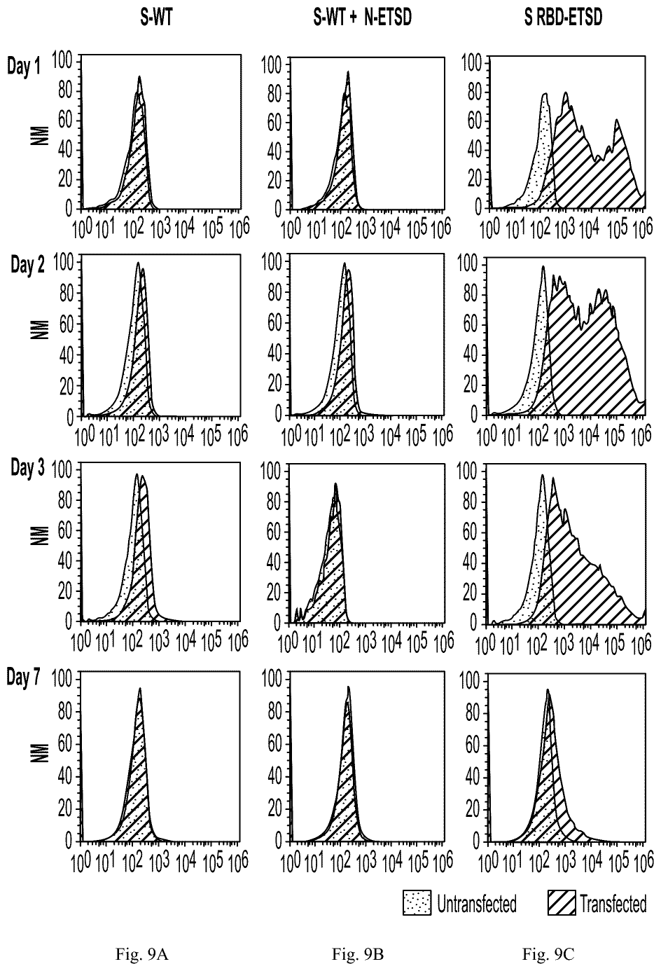

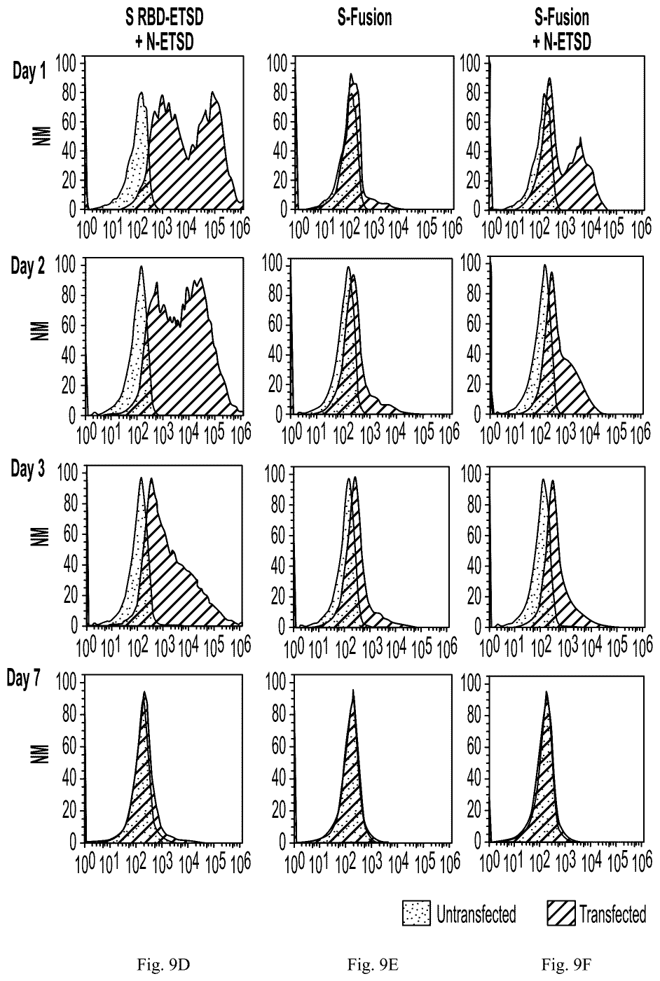

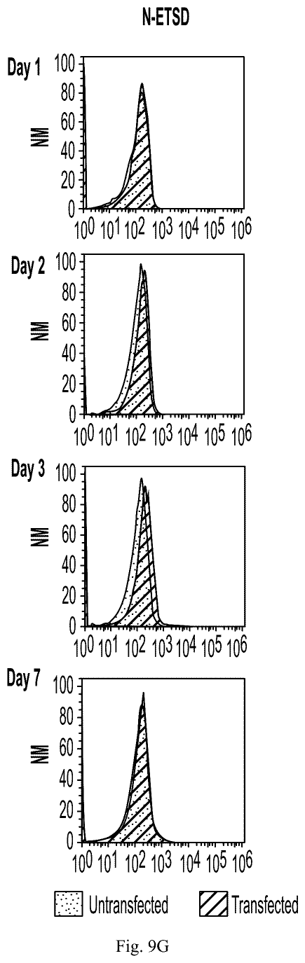

As shown in FIG. 9, anti-RBD-specific antibodies did not detect RBD on the surface of HEK 293T cells transfected with hAd5 S-WT (FIG. 9a) or hAd5 S-WT+N-ETSD (FIG. 9b) constructs, while hAd5 S-Fusion alone was slightly higher (FIG. 9e). As expected, both constructs with RBD, hAd5 RBD-ETSD and RBD-ETSD+N-ETSD, showed high binding of anti-RBD antibody (FIGS. 9c and d). Notably, high cell-surface expression of RBD was detected after transfection with bivalent hAd5 S-Fusion+N-ETSD (FIG. 9f). These findings support our proposition that an hAd5 S-Fusion+N-ETSD construct, containing a high number and variety of antigens provided by both full-length, optimized S with proper folding and N leads to enhanced expression and cell surface display of RBD in a vaccine construct.

### Example 8: Immunoblot Correlation of Enhanced S Expression with hAd5 S-Fusion+N-ETSD

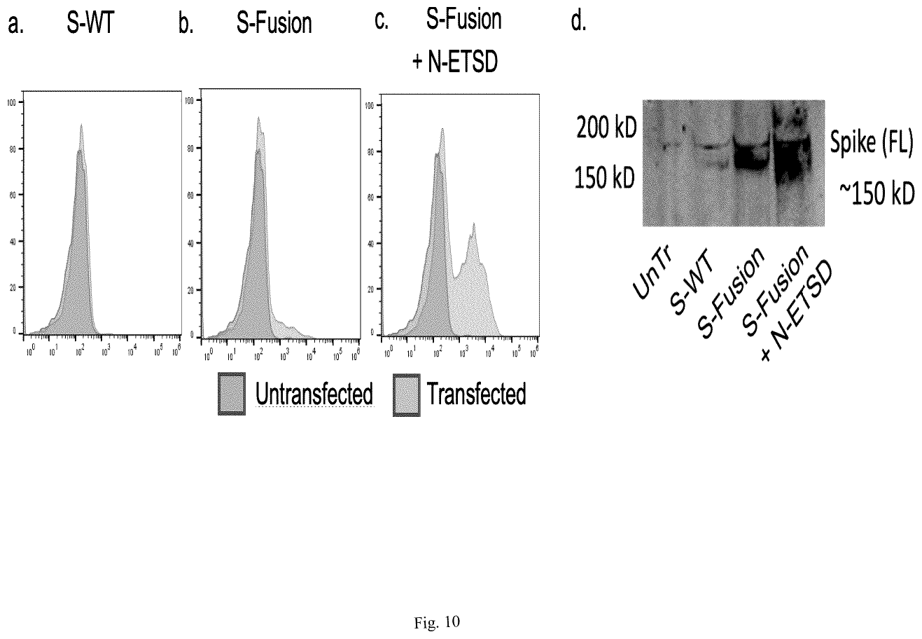

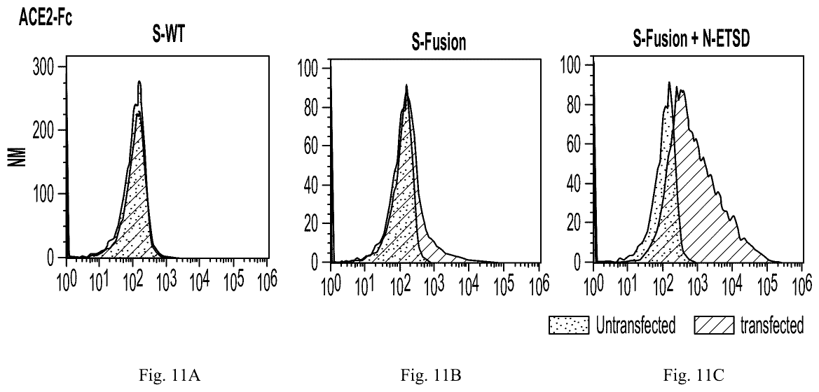

Immunoblot analysis of S expression correlated with enhanced S expression (FIG. 10), showing again that the bivalent hAd5 S-Fusion+N-ETSD construct enhances expression of S compared to S-Fusion alone.

### Example 9: Confirmation of Native Folding of Enhanced Surface RBD Following hAd5 S-Fusion+N-ETSD Transfection

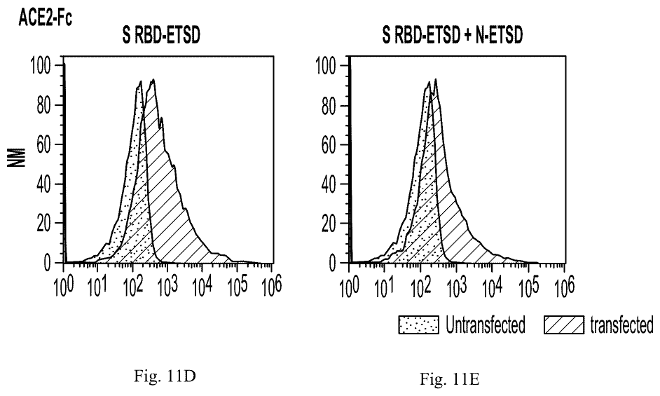

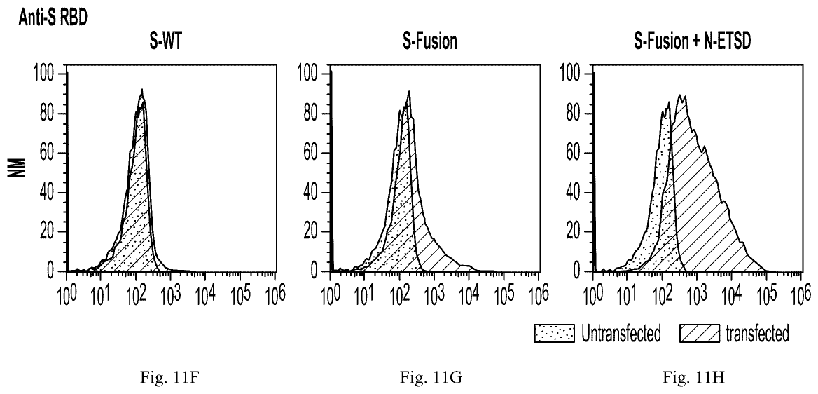

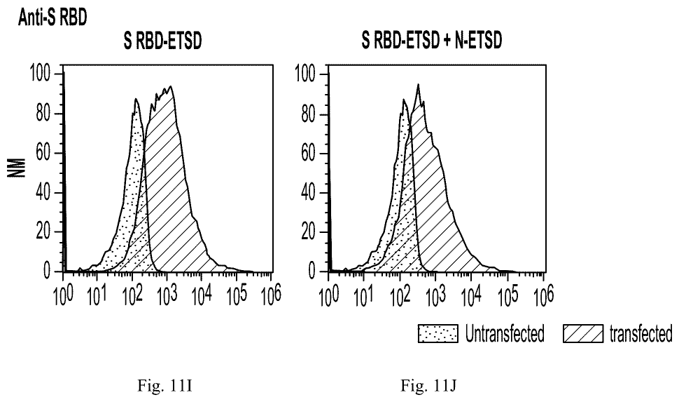

Determination of the binding of recombinant ACE2-Fc was performed to confirm the native, physiologically-relevant folding of the S RBD after expression from the hAd5 S-Fusion+N-ETSD vaccine candidate. S RBD binds ACE2 during the course of SARS-CoV-2 infection and an effective neutralizing antibody prevents this interaction and thus infection. Such a neutralizing antibody is more likely to be effective if raised in response to S presented in the correct conformation. In addition to enhancement of cell surface expression, the optimized S allows for proper protein folding. It was found that compared to either hAd5 S-WT or hAd5 S-Fusion (FIG. 11 a and b, respectively), ACE2-Fc binding to S RBD expressed from the hAd5 S-Fusion+N-ETSD was clearly enhanced (FIG. 11c). Anti-RBD antibody binding studies (FIG. 11f-j) performed with the same experiment, confirmed the enhanced surface expression findings noted by ACE2-Fc binding. These findings of conformationally correct and enhanced S RBD expression, important for production of neutralizing antibodies, directed us to elect the hAd5 S-Fusion+N-ETSD vaccine candidate for clinical trials.

### Example 10: hAd5 N-ETSD Successfully Directs N to an Endosomal/Lysosomal Compartment

The ETSD design successfully translocated N to the endosomal subcellular compartment. After infection of HeLa cells with N-ETSD, N co-localized with the endosomal marker 45 transferrin receptor (CD71), as shown in FIG. 12c, and also co-localized with the lysosomal marker Lamp1 (FIG. 12d), demonstrating that N-ETSD is translocated throughout the endosomal pathway to lysosomes, enabling processing for MHC II presentation. N-wild type (N-WT), compared to N-ETSD, shows diffuse cytoplasmic distribution and does not co-localize with the lysosomal marker (FIG. 12e). These findings confirm the role of the ETSD in directing N to an endosomal/lysosomal compartment that will result in increased MHC II presentation and CD4+ activation by N.

### Example 11: In Vivo hAd5 S-Fusion+N-ETSD Vaccine Immunogenicity Studies

Based on the evidence that S-Fusion+N-ETSD resulted in enhanced expression of physiologically-relevant RBD and that N-ETSD successfully translocated to the endosomal/lysosomal compartment, the bivalent hAd5 S-Fusion+N-ETSD vaccine was chosen for inoculation of 7-week old female CD-1 mice. The unique properties of this construct would result in the generation of both CD8+ and CD4+ T-cell responses and neutralizing antibodies. As described in Methods, mice received an initial injection on Day 0 and a second injection on Day 21. Sera were collected on Day 0 and at the end of the study on Day 28 for antibody and neutralization analyses. Splenocytes were also collected on Day 28 for intracellular cytokine staining (ICS) and ELISpot analyses. All age- and gender-matched animals assigned to the study appeared normal with no site reactions and no loss of body weight throughout the dosing were seen, consistent with previous observations with the hAd5 [E1-, E2b-, E3-] platform

### Example 12: hAd5 S-Fusion+N-ETSD Generates Both CD8□+ and CD4+ T-Cell Responses

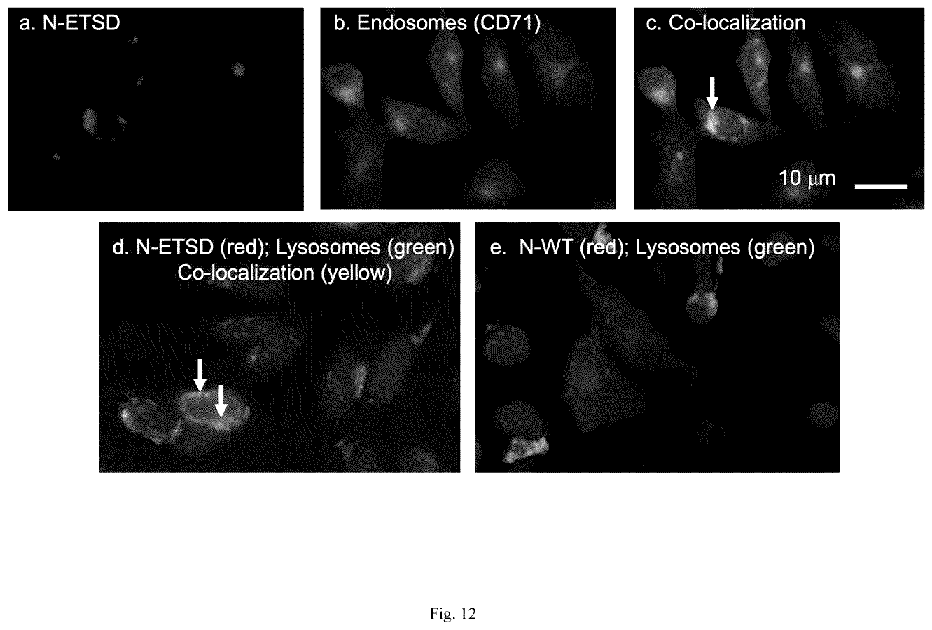

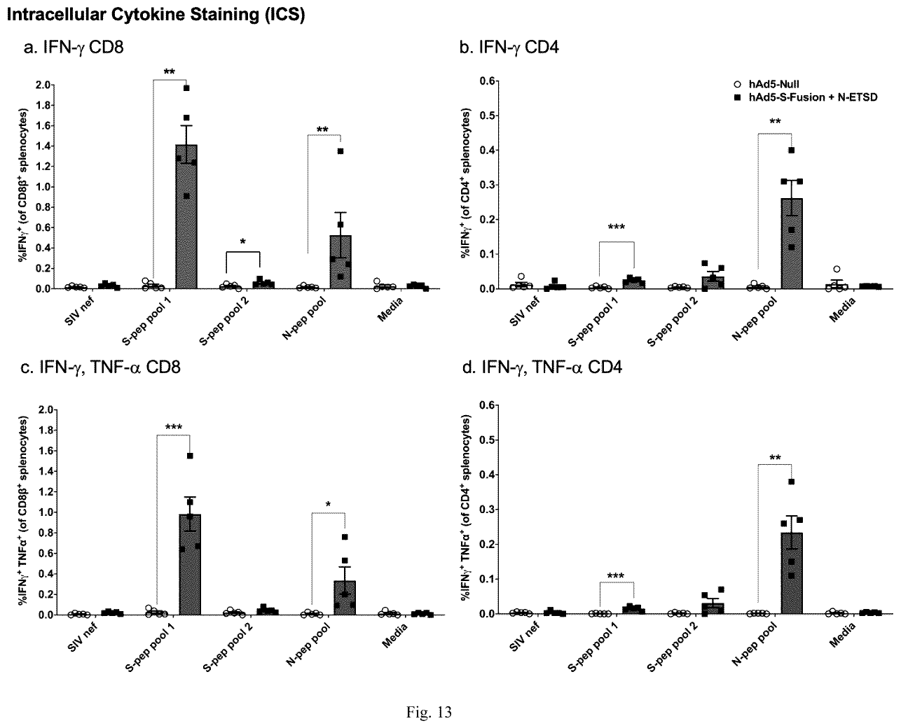

CD8+ activation by both S and N: CD8β+ splenocytes from hAd5 S-Fusion+N-ETSD vaccinated mice exposed to S peptide pool 1 (containing RBD and S1) show IFN-γ expression that is significantly higher compared to hAd5 null mice (FIG. 13a); splenocytes from these mice also expressed intracellular IFN-γ in response to the N peptide pool. Evaluation of simultaneous IFN-γ/TNF-α expression from CD8 β+ splenocytes (FIG. 13c) mirrored those for IFN-γ expression alone. These results indicate that both S and N activate CD8+ T cells.

CD4+ activation by N: Although CD8+ cytotoxic T cells mediate killing of virus infected cells, CD4+ T cells are required for sustained cytotoxic T lymphocyte (CTL) activity. Thus, CD4+ T cells in the vaccinated animals was evaluated. In contrast to CD8 β+ splenocytes, only the N peptide pool stimulated CD4+ splenocytes from hAd5 S-Fusion+N-ETSD-inoculated mice to express IFN-γ (FIG. 13b) or IFN-γ/TNF-α (FIG. 13d) at levels that were substantially higher than hAd5 Null control. The contribution by N of CD4+ T-cell responses is vital to an effective immune response to the candidate vaccine.

### Example 13: hAd5 S-Fusion+N-ETSD Generates Antibody Responses to Both S and N Antigens

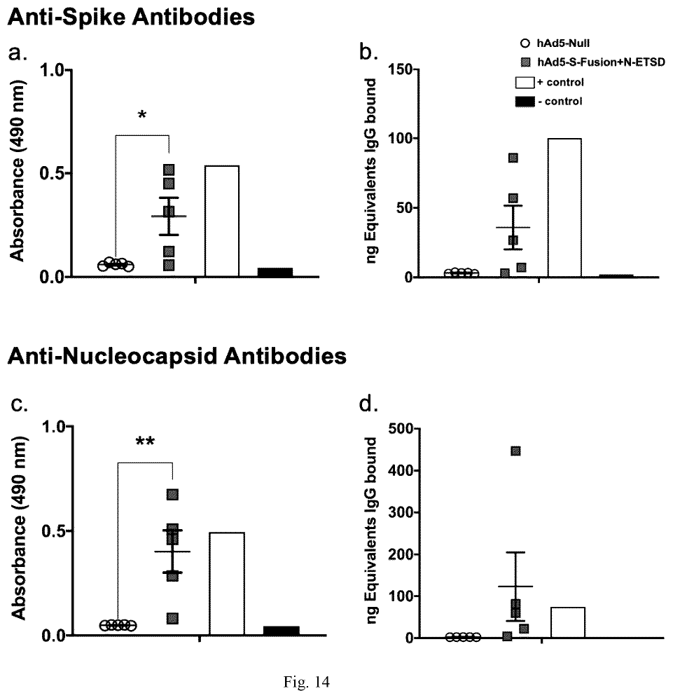

The primary objective of coronavirus vaccines currently in development are neutralizing antibodies against spike, thus we examined antibody production in mice vaccinated with our bivalent vaccine. There was significant production of both anti-S (FIG. 14a) and anti-N (FIG. 14c) antibodies in the sera from CD-1 mice vaccinated with hAd5 S-Fusion+N-ETSD at Day 28 in the study. Compared to anti-S antibodies, anti-N antibodies were higher in sera, given the dilution factor for sera was 1:90 for anti-N antibody analysis and 1:30 for anti-S antibody analysis.

A standard curve of IgG was generated, then absorbance values were converted into mass equivalents for both anti-S and anti-N antibodies (FIGS. 14b and d). These values were used to calculate that hAd5 S-Fusion+NETSD vaccination generated a geometric mean value of 5.8 μg S-specific IgG and 42 μg N-specific IgG per mL of serum, therefore the relative μg amount of anti-N antibodies is higher than that for anti-S antibodies and reflects the strong contribution of N to anti-SARS-CoV-2 antibody production.

### Example 14: hAd5 S-Fusion+N-ETSD Vaccine Generates Potent Neutralizing Antibodies as Assessed by Both cPass and Live Virus Neutralization Assays

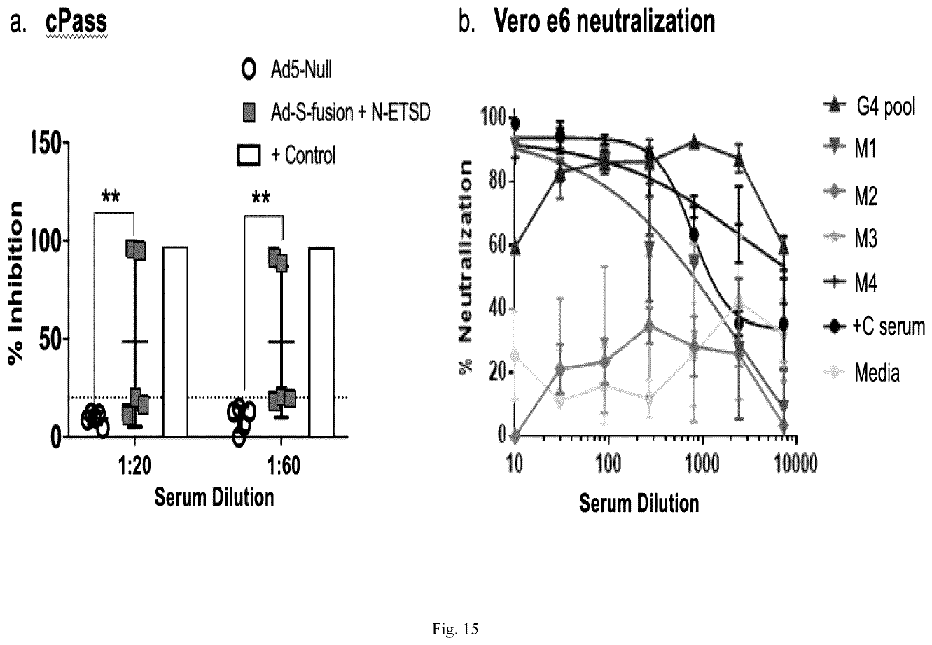

Neutralizing antibody activity was evaluated using a cell free assay (cPass) as well as live virus infection in vitro. As seen in FIG. 15a, the cPass assay showed inhibition of S RBD:ACE2 binding for all mice and ˜100% inhibition for two mice at both dilutions of 1:20 and 1:60. The Vero E6 neutralization assay results are shown for the four mice that showed S-specific antibodies by ELISA. The high persistent neutralization seen even at the high dilution factors suggests the intriguing possibility that the bivalent, multi-antigen, multi-epitope generation by hAd5 S-Fusion+N-ETSD vaccine, could result in synergies of neutralizing immune responses (FIG. 15b); at epitopes in addition to those associated with RBD-ACE2 binding. As can be seen in FIG. 15b, the value for 50% neutralization (IC50) is present at 1:10,000 serum dilution for the G4 pool of sera from mice that showed S-specific antibodies, ten times higher than the convalescent serum with a dilution of 1:1,000. The potent neutralization, confirmed by two assays, supports the predicted efficacy of the hAd5 S-Fusion+ETSD vaccine candidate and its advancement to clinical trials

### Example 15: hAd5 S-Fusion+N-ETSD Generates Th1 Dominant Responses Both in Humoral and T-Cell Immunity

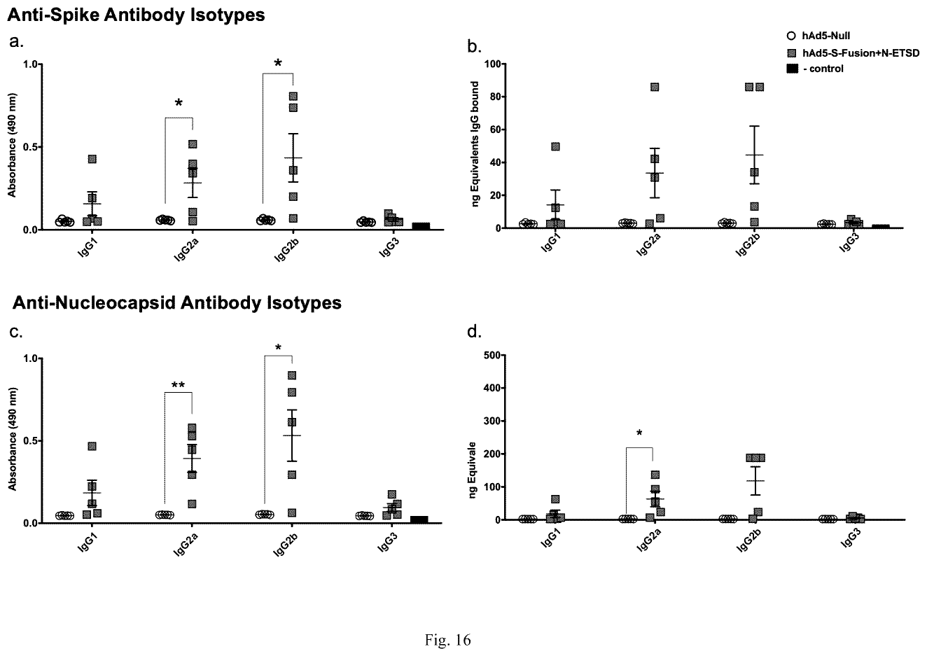

Antibody Th1 dominance in response to N and S: IgG2a, IgG2b, and IgG3 represent Th1 dominance; while IgG1 represents Th2 dominance. For both anti-S (FIG. 16a) and anti-N (FIG. 16c) antibodies in sera from hAd5 S-Fusion+N-ETSD vaccinated mice, IgG2a and IgG2b isotypes were predominant and significantly higher compared to the hAd5 Null control. These data show the Th1 dominance of antibody production in response to the hAd5 S-Fusion+N-ETSD vaccine

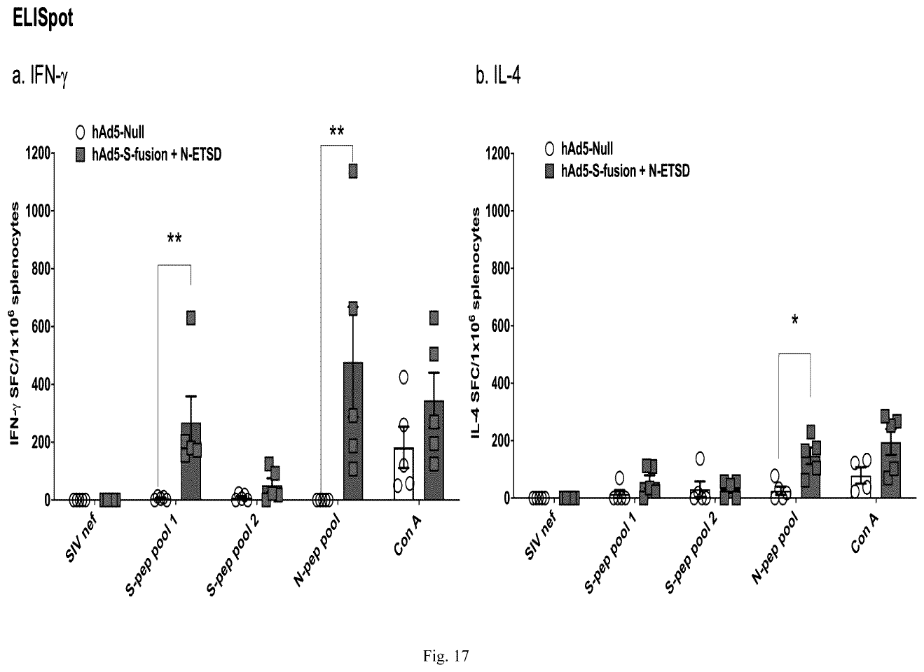

T-cell Th1 dominance in response to N and S: IFN-γ production correlates with CTL activity 47 (Th1 dominance), whereas, IL-4 causes delayed viral clearance 48 (Th2 dominance). A ratio of IFN-γ to IL-4 of 1 is balanced and a ratio greater than 1 is demonstrative of Th1 dominance. Thus, we examined IFN-γ and IL-4 production in animals immunized with the bivalent S plus N vaccine. As determined by ELISpot, IFN-γ secretion was significantly higher for hAd5 S-Fusion+N-ETSD than for hAd5 Null splenocytes in response to both S peptide pool 1 and the N peptide pool (FIG. 17a), but IL-4 was only secreted at significantly higher levels for hAd5 S-Fusion+N-ETSD in response to the N peptide pool (FIG. 17b).

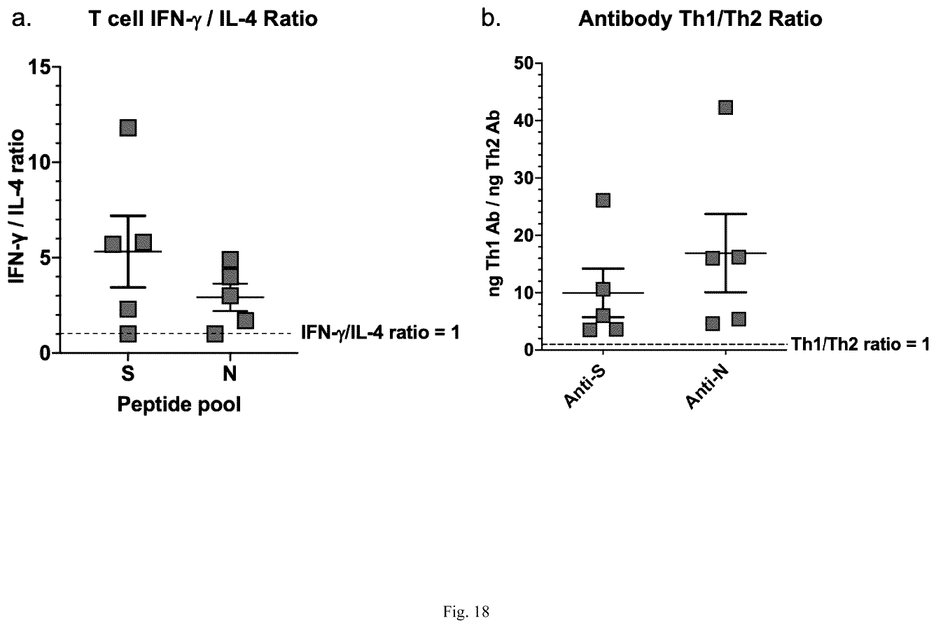

The Th1-type predominance is also seen when the ratio of IFN-□ to IL-4 based on spot forming units in response to the combined S peptide pools and the N peptide pool, is considered (FIG. 18a).

Th1 predominance was seen again in humoral responses, where the ratio based on ng equivalence of Th1 related antibodies (IgG2a, IgG2b, and IgG3) to Th2 related antibodies (IgG1) for both anti-S and anti-N antibodies is greater than 1 in all mice (FIG. 18b).

This Th1 dominant profile of the hAd5 S-Fusion+N-ETSD vaccine candidate provides further justification for hAd5 S-Fusion+N-ETSD to be our lead candidate for clinical testing

The hAd5 S-Fusion+N-ETSD vaccine was designed to overcome the risks of an S-only vaccine and elicit both T-cell immunity and neutralizing antibodies, leveraging the vital role T cells play in generating long-lasting antibody responses and in directly killing infected cells. Both CD4+ and CD8+ T cells are multifunctional, and induction of such multifunctional T cells by vaccines correlated with better protection against infection. We posit that enhanced CD4+ T-cell responses and Th1 predominance resulting from expression of an S antigen optimized for surface display and an N antigen optimized for endosomal/lysosomal subcellular compartment localization and thus MHC I and II presentation, led to increased dendritic cell presentation, cross-presentation, B cell activation, and ultimately high neutralization capability. Furthermore, the potent neutralization capability at high dilution seen for the pooled sera from hAd5 S-Fusion+N-ETSD vaccinated mice, combined with Th1 dominance of antibodies generated in response to both S and N antigens, supports the objective of this vaccine design.

Contemporaneous MHC I and MHC II presentation of an antigen by the antigen presenting cell activates CD4+ and CD8+ T cells simultaneously and is optimal for the generation of memory B and T cells. A key finding of our construct is that N-ETSD, which we show is directed to the endosomal/lysosomal compartment, elicits a CD4+ response, a necessity for induction of memory T cells and helper cells for B cell antibody production. Others have also reported on the importance of lysosomal localization for eliciting the strongest T-cell IFN-γ and CTL responses, compared to natural N.50,51

The T-cell responses to the S and N antigens expressed by hAd5 S-Fusion+N-ETSD were polycytokine, including IFN-g and TNF-α, consistent with successful antimicrobial immunity in bacterial and viral infections. Post-vaccination polycytokine T-cell responses have been shown to correlate with vaccine efficacy, including those with a viral vector. Highly relevant here, polycytokine T-cell responses to SARS-CoV-2 N protein are consistent with recovered COVID-19 patients, suggesting that the bivalent hAd5 S-Fusion+N-ETSD vaccine will provide vaccine subjects with greater protection against SARS-CoV-2.

In contrast to N, the S protein, here expressed as S-Fusion with confirmed enhanced RBD cell-surface expression and conformational integrity as evidenced by high ACE2-Fc binding, generated predominantly CD8+ T cells. Our results confirmed our vaccine design goal, showing that S-Fusion induced elevated levels of antigen-specific T-cell responses against S compared to S-WT. To ensure MHC presentation to both MHC I (for CD8+ T-cell activation) and MHC II (for CD4+ T-cell activation), it is necessary to vaccinate with both S and N antigens optimized to produce this coordinated response.

The neutralization data with live SARS-CoV-2 virus demonstrated the potency of the antibody response generated following vaccination with hAd5 S-Fusion+N-ETSD, with evidence of high neutralization even at a high dilution factor. In addition, a striking synergistic effect of pooled sera was evident, with potent neutralization even greater than control convalescent serum at >1:1,000 dilution.

The hAd5 S-Fusion+N-ETSD construct described above is delivered by a next generation hAd5 [E1-, E2b-, E3-] platform wherein the E2b deletion (pol) alone enables prolonged transgene production and allows homologous vaccination (prime and the boost formulation is the same) in the presence of pre-existing adenoviral immunity.38 In addition to the generation of cellular and humoral immunity by the subcutaneous injection of hAd5 S-Fusion+N-ETSD, we are also exploring the potential of inducing IgA mucosal immunity by utilizing the same vaccine in an oral or sublingual formulation in clinical trials.

### Example 16: Methods

The hAd5 [E1-, E2b-, E3-] Platform and Constructs

For studies herein, the 2nd generation hAd5 [E1-, E2b-, E3-] vector was used (FIG. 1c) to create viral vaccine candidate constructs. hAd5 [E1-, E2b-, E3-] backbones containing SARS-CoV-2 antigen expressing inserts and virus particles were produced as previously described.37 In brief, high titer adenoviral stocks were generated by serial propagation in the E1- and E2b-expressing E.C7 packaging cell line, followed by CsCl2 purification, and dialysis into storage buffer (2.5% glycerol, 20 mM Tris pH 8, 25 mM NaCl) by ViraQuest Inc. (North Liberty, IA). Viral particle counts were determined by sodium dodecyl sulfate disruption and spectrophotometry at 260 and 280 nm and viral titers were determined using the Adeno-X™ Rapid Titer Kit (Takara Bio). The constructs created included:
S-WT: S protein comprising 1273 amino acids and all S domains: extracellular (1-1213), transmembrane (1214-1234), and cytoplasmic (1235-1273) (Unitprot PODTC2);
S RBD-ETSD: S Receptor Binding Domain (S RBD) with an ETSD;
N-ETSD: Nucleocapsid (N) with ETSD;
S-WT+N-ETSD: S-WT with an Enhanced T-cell Stimulation Domain (ETSD);
S-RBD-ETSD+N-ETSD;
S Fusion: S optimized to enhance surface expression and display of RBD; and Bivalent S-Fusion+N-ETSD;
   Transfection of HEK 293T Cells with hAd5 Constructs

To determine surface expression of the RBD epitope by vaccine candidate constructs, we transfected HEK 293T cells with hAd5 construct DNA and quantified surface RBD by flow cytometric detection using anti-RBD antibodies. There were seven constructs tested: S-WT, S-WT+N-ETSD, S RBD-ETSD, S RBD-ETSD+N-ETSD, S-Fusion, S-Fusion+N-ETSD, and N-ETSD. HEK 293T cells (2.5×10⁵ cells/well in 24 well plates) were grown in DMEM (Gibco Cat #11995-065) with 10% FBS and 1×PSA (100 units/mL penicillin, 100 μg/mL streptomycin, 0.25 ug/mL Amphotericin B) at 37° C. Cells were transfected with 0.5 μg of hAd5 plasmid DNA using a JetPrime transfection reagent (Polyplus Catalog #89129-924) according to the manufacturer's instructions. Cells were harvested 1, 2, 3, and 7 days post transfection by gently pipetting cells into medium and labeled with an anti-RBD monoclonal antibody (clone D003 Sino Biological Catalog #40150-D003) and F(ab′)2-Goat anti-Human IgG-Fc secondary antibody conjugated with R-phycoerythrin (ThermoFisher Catalog #H10104). Labeled cells were acquired using a Thermo-Fisher Attune NxT flow cytometer and analyzed using Flowjo Software.

Immunocytochemical Labeling of hAd5 Infected HeLa Cells

To determine subcellular localization of N after infection or transfection of HeLa cells with hAd5 N-wild type (WT) or hAd5 N-ETSD (each with a flag tag to allow labeling), 48 hours after infection or transfection cells were fixed with 4% paraformaldehyde (PFA) and permeabilized with 0.4% Triton ×100, in PBS) for 15 min. at room temperature. To label N, cells were then incubated with an anti-flag monoclonal (Anti-Flag M2 produced in mouse, Sigma cat #F1804) antibody at 1:1000 in phosphate buffered saline with 3% BSA overnight at 4° C., followed by washes in PBS and a 1 hour incubation with a goat anti-Mouse IgG (H+L) Highly Cross-Adsorbed Secondary Antibody, Alexa Fluor Plus 555 (Life Technologies, Cat #A32727) at 1:500. For co-localization studies, cells were also incubated overnight at 4° C. with a sheep anti-Lamp1 Alexa Fluor 488-conjugated (lysosomal marker) antibody (R&D systems, Cat #IC7985G) at 1:10 or a rabbit anti-CD71 (transferrin receptor, endosomal marker) antibody (ThermoFisher Cat #PA5-83022) at 1:200. After removal of the primary antibody, two washes in PBS and three 3 washes in PBS with 3% BSA, cells were incubated with fluor-conjugated secondary antibodies when applicable at 1:500 (Goat anti-Rabbit IgG (H+L) Highly Cross-Adsorbed Secondary Antibody, Alexa Fluor 488, Life technologies, A-11034) for 1 hour at room temperature. After brief washing, cells were mounted with Vectashield Antifade mounting medium with DAPI (Fisher Scientific, Cat #NC9524612) and immediately imaged using a Keyence all-in-one Fluorescence microscope camera and Keyence software.

### Immunoblot Analysis of S Antigen Expression

HEK 293T cells transfected with hAd5 S-WT, S-Fusion, or S-Fusion+N-ETSD constructs were cultured and transfected as described in the main manuscript and harvested 3 days after transfection in 150 mL RIPA lysis buffer with 1× final Protease Inhibitor cocktail (Roche). After protein assay, equivalent amounts of total protein were loaded into and run on a 4 to 12% gradient polyacrylamide gel (type) and transferred to nitrocellulose membranes using semi-dry transfer apparatus. Anti-Spike S2 (SinoBiological Cat #40590-T62) was used as the primary antibody and IRDye® 800CW Goat anti-Rabbit IgG (H+L) (Li-Cor, 925-32211) as the secondary antibody using the Ibind Flex platform. Antibody-specific signals were detected with an infrared Licor Odyssey instrument.

ACE2-IgG1Fc Binding to hAd5 Transfected HEK 293T Cells

HEK 293T cells were cultured at 37° C. under conditions described above for transfection with hAd5 S-WT, S-Fusion, S-Fusion+N-ETSD, S RBD-ETSD, or S RBD-ETSD+N-ETSD and were incubated for 2 days and harvested for ACE2-Fc binding analysis. Recombinant ACE2-IgG1Fc protein was produced using Maxcyte transfection in CHO-S cells that were cultured for 14 days. ACE2-IgG1Fc was then purified using a MabSelect SuRe affinity column on AKTA Explorer.

Purified ACE2-IgG1Fc was dialyzed into 10 mM HEPES, pH7.4, 150 mM NaCl and concentrated to 2.6 mg/mL. For binding studies, the ACE2-IgG1Fc was used at a concentration of 1 □g/mL for binding. Cells were incubated with ACE2-Fc for 20 minutes and, after a washing step, were then labeled with a PE conjugated F(ab′)2-goat anti-human IgG Fc secondary antibody at a 1:100 dilution, incubated for 20 minutes, washed and acquired on flow cytometer. Histograms are based on normalized mode (NM) of cell count-count of cells positive for signal in PE channel.

Vaccination of CD-1 Mice with the hAd5 S-Fusion+N-ETSD Vaccine Candidate

CD-1 female mice (Charles River Laboratories) 7 weeks of age were used for immunological studies performed at the vivarium facilities of Omeros Inc. (Seattle, WA). After an initial blood draw, mice were injected with either hAd5 Null (a negative control) or vaccine candidate hAd5 S-Fusion+N-ETSD on Day 0 at a dose of 1×1010 viral particles (VP). There were 5 mice per group. Mice received a second vaccine dose on Day 21 and on Day 28, blood was collected via the submandibular vein from isoflurane-anesthetized mice for isolation of sera and then mice were euthanized for collection of spleen and other tissues.

### Splenocyte Collection and Intracellular Cytokine Staining (ICS)

Spleens were removed from each mouse and placed in 5 mL of sterile medium of RPMI (Gibco Cat #22400105), HEPES (Hyclone Cat #SH30237.01), 1×Pen/Strep (Gibco Cat #15140122), and 10% FBS (Gibco Cat #16140-089). Splenocytes were isolated within 2 hours of collection. ICS for flow cytometric detection of CD8β+ and CD4+ T-cell-associated IFN-γ and IFN-γ/TNFα+ production in response to stimulation by S and N peptide pools.

Stimulation assays were performed using 106 live splenocytes per well in 96-well U-bottom plates. Splenocytes in RPMI media supplemented with 10% FBS were stimulated by the addition of peptide pools at 2 g/mL/peptide for 6 h at 37° C. in 5% CO2, with protein transport inhibitor, GolgiStop (BD) added two hours after initiation of incubation. Stimulated splenocytes were then stained for lymphocyte surface markers CD8□ and CD4, fixed with CytoFix (BD), permeabilized, and stained for intracellular accumulation of IFN-γ and TNF-α. Fluorescent-conjugated antibodies against mouse CD8□ antibody (clone H35-17.2, ThermoFisher), CD4 (clone RM4-5, BD), IFN-γ (clone XMG1.2, BD), and TNF-α (clone MP6-XT22, BD) and staining was performed in the presence of unlabeled anti-CD16/CD32 antibody (clone 2.4G2). Flow cytometry was performed using a Beckman-Coulter Cytoflex S flow cytometer and analyzed using Flowjo Software.

### ELISpot Assay

ELISpot assays were used to detect cytokines secreted by splenocytes from inoculated mice. Fresh splenocytes were used on the same day, as were cryopreserved splenocytes containing lymphocytes. The cells (2-4×105 cells per well of a 96-well plate) were added to the ELISpot plate containing an immobilized primary antibodies to either IFN-γ or IL-4 (BD), and were exposed to various stimuli (e.g. control peptides, target peptide pools/proteins) comprising 2 g/mL peptide pools or 10 g/mL protein for 36-40 hours. After aspiration and washing to remove cells and media, extracellular cytokine was detected by a secondary antibody to cytokine conjugated to biotin (BD). A streptavidin/horseradish peroxidase conjugate was used detect the biotin-conjugated secondary antibody. The number of spots per well, or per 2-4×105 cells, was counted using an ELISpot plate reader.

### ELISA for Detection of Antibodies

For antibody detection in sera from inoculated mice, ELISAs specific for spike and nucleocapsid antibodies, as well as for IgG subtype (IgG1, IgG2a, IgG2b, and IgG3) antibodies were used. A microtiter plate was coated overnight with 100 ng of either purified recombinant SARS-CoV-2 S-FTD (full-length S with fibritin trimerization domain, constructed and purified in-house by ImmunityBio), SARS-CoV-2 S RBD (Sino Biological, Beijing, China; Cat #401591-V08B1-100) or purified recombinant SARS-CoV-2 nucleocapsid (N) protein (Sino Biological, Beijing, China; Cat #40588-V08B) in 100 μL of coating buffer (0.05 M Carbonate Buffer, pH 9.6). The wells were washed three times with 250 μL PBS containing 1% Tween 20 (PBST) to remove unbound protein and the plate was blocked for 60 minutes at room temperature with 250 μL PBST. After blocking, the wells were washed with PBST, 100 μL of diluted serum samples were added to wells, and samples incubated for 60 minutes at room temperature. After incubation, the wells were washed with PBST and 100 μL of a 1/5000 dilution of anti-mouse IgG HRP (GE Health Care; Cat #NA9310V), or anti-mouse IgG1 HRP (Sigma; Cat #SAB3701171), or anti-mouse IgG2a HRP (Sigma; Cat #SAB3701178), or anti-mouse IgG2b HRP (Sigma; catalog #SAB3701185), or anti-mouse IgG3 HRP conjugated antibody (Sigma; Cat #SAB3701192) was added to wells. For positive controls, a 100 μL of a 1/5000 dilution of rabbit anti-N IgG Ab or 100 μL of a 1/25 dilution of mouse anti-S serum (from mice immunized with purified S antigen in adjuvant) were added to appropriate wells. After incubation at room temperature for 1 hour, the wells were washed with PBS-T and incubated with 200 μL o-phenylenediamine-dihydrochloride (OPD substrate (Thermo Scientific Cat #A34006) until appropriate color development. The color reaction was stopped with addition of 50 μL 10% phosphoric acid solution (Fisher Cat #A260-500) in water and the absorbance at 490 nm was determined using a microplate reader (SoftMax® Pro, Molecular Devices).

### Calculation of Relative g Amounts of Antibodies

A standard curve of IgG was generated and absorbance values were converted into mass equivalents for both anti-S and anti-N antibodies. Using these values, we were able to calculate that hAd5 S-Fusion+N-ETSD vaccination generated a geometric mean value of 5.8 μg S-specific IgG and 42 μg N-specific IgG per milliliter of serum.

cPass™ Neutralizing Antibody Detection

The GenScript cPass™ (https://www.genscript.com/cpass-sars-cov-2-neutralization-antibody-detection-Kit.html) for detection of neutralizing antibodies was used according to the manufacturer's instructions.44 The kit detects circulating neutralizing antibodies against SARS-CoV-2 that block the interaction between the S RBD with the ACE2 cell surface receptor. It is suitable for all antibody isotypes and appropriate for use with in animal models without modification.

### Vero E6 Cell Neutralization Assay

All aspects of the assay utilizing virus were performed in a BSL3 containment facility according to the ISMMS Conventional Biocontainment Facility SOPs for SARS-CoV-2 cell culture studies. Vero e6 kidney epithelial cells from Cercopithecus aethiops (ATCC CRL-1586) were plated at 20,000 cells/well in a 96-well format and 24 hours later, cells were incubated with antibodies or heat inactivated sera previously serially diluted in 3-fold steps in DMEM containing 2% FBS, 1% NEAAs, and 1% Pen-Strep; the diluted samples were mixed 1:1 with SARS-CoV-2 in DMEM containing 2% FBS, 1% NEAAs, and 1% Pen-Strep at 10,000 TCID 50/mL for 1 hr. at 37° C., 5% CO2. This incubation did not include cells to allow for neutralizing activity to occur prior to infection. The samples for testing included sera from the four mice that showed >20% inhibition of ACE2 binding in cPass, pooled sera from those four mice, sera from a COVID-19 convalescent patient, and media only. For detection of neutralization, 120 μL of the virus/sample mixture was transferred to the Vero E6 cells and incubated for 48 hours before fixation with 4% PFA. Each well received 60 μL of virus or an infectious dose of 600 TCID50. Control wells including 6 wells on each plate for no virus and virus-only controls were used. The percent neutralization was calculated as 100−((sample of interest-[average of “no virus” ])/[average of “virus only” ])*100) with a stain for CoV-2 Np imaged on a Celigo Imaging Cytometer (Nexcelom Bioscience).

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the disclosures herein, and does not pose a limitation on the scope of the invention otherwise claimed. No language in the specification should be construed as indicating any non-claimed element essential to the practice of the claimed invention.

Many more modifications besides those already described are possible without departing from the concepts disclosed herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A nucleic acid encoding a chimeric protein comprising 1) a CoV2 nucleocapsid (N) protein having the amino acid sequence of SEQ ID NO: 1 and 2) an endosomal targeting sequence (ETSD) having the amino acid sequence of SEQ ID NO: 2.

2. The nucleic acid of claim 1, wherein the nucleic acid portion has a nucleotide sequence of SEQ ID NO:3.

3. The nucleic acid of claim 1, wherein the nucleic acid further comprises a co-stimulatory molecule and/or an immune stimulatory cytokine.

4. The nucleic acid of claim 3, wherein the co-stimulatory molecule is selected from the group consisting of CD80, CD86, CD30, CD40, CD30L, CD40L, ICOS-L, B7-H3, B7-H4, CD70, OX40L, 4-IBBL, GITR-L, TIM-3, TIM-4, CD48, CD58, TLIA, ICAM-1, and LFA3.

5. The nucleic acid of claim 3, wherein the immune stimulatory cytokine is selected from the group consisting of IL-2, IL-12, IL-15, nogapendekin alfa-imbakicept, IL-21, IPSI, and LMP1.

6. A vaccine composition comprising the nucleic acid of claim 1, wherein the composition is formulated for injection.

7. A method for inducing immunity against CoV2 in a patient in need thereof, the method comprising administering to the patient the vaccine composition of claim 6.

8. A replication defective adenovirus, wherein the adenovirus comprises:
a. an E1 gene region deletion;
b. an E2b gene region deletion; and
c. the nucleic acid of claim 1.

9. A yeast or lysate thereof, wherein the yeast comprises the nucleic acid of claim 1.

10. A replication defective adenoviral vector comprising the following components: an E1 gene region deletion; an E2b gene region deletion; and a nucleic acid portion that encodes a chimeric protein comprising a SARS-CoV-2 N protein and an endosomal targeting sequence.

11. The replication defective adenoviral vector of claim 10, wherein the nucleic acid portion that encodes the chimeric protein is depicted by SEQ ID NO:3.

12. The replication defective adenoviral vector of claim 10, further comprising a trafficking sequence, a co-stimulatory molecule, and/or an immune stimulatory cytokine.

13. The replication defective adenoviral vector of claim 10, wherein the co-stimulatory molecule is selected from the group consisting of CD80, CD86, CD30, CD40, CD30L, CD40L, ICOS-L, B7-H3, B7-H4, CD70, OX40L, 4-IBBL, GITR-L, TIM-3, TIM-4, CD48, CD58, TLIA, ICAM-1, and LFA3.

14. The replication defective adenoviral vector of claim 10, wherein the immune stimulatory cytokine is selected from the group consisting of IL-2, IL-12, IL-15, nogapendekin alfa-imbakicept, IL-21, IPSI, and LMP1.

15. A method for inducing immunity against a coronavirus in a patient in need thereof, the method comprising administering to the subject an immunotherapy composition comprising a recombinant entity, wherein the recombinant entity comprises a nucleic acid that encodes a nucleocapsid protein of coronavirus 2 (CoV2) and an endosomal targeting sequence.

16. The method of claim 15, wherein the recombinant entity comprises a replication defective adenoviral vector comprising an E1 gene region deletion and an E2b gene region deletion.

17. The method of claim 15, wherein an immune stimulatory cytokine is administered.

18. The method of claim 17, wherein the immune stimulatory cytokine is selected from the group consisting of IL-2, IL-12, IL-15, IL-15 super agonist (N803), IL-21, IPS 1, and LMP 1.

19. The method of claim 15, wherein the coronavirus is SARS-CoV-2.

20. The method of claim 15, wherein the coronavirus is not SARS-CoV-2.
